SEQUENCE LISTING (2 sequences)

SEQ ID NO: 1 - protein, 31 aa, synthetic construct
SIIGIIMGILGNIPQVIQIIMSIVKAFKGNK

SEQ ID NO: 2 - protein, 31 aa, synthetic construct
IFKIIISKIQIVMGLNGIPIKVAGSQNIGMI

(19) United States
(12) Patent Application Publication
(10) Pub. No.: US 2024/0350491 A1
(43) Pub. Date: Oct. 24, 2024

(54) METHODS AND COMPOSITIONS FOR IMMUNE CONTROL THROUGH DICKKOPF-1

(71) Applicant: Baylor College of Medicine, Houston, TX (US)

(72) Inventors: John Knight, Houston, TX (US); Yifan Wu, Houston, TX (US); David Corry, Houston, TX (US)

(21) Appl. No.: 18/688,164

(22) PCT Filed: Sep. 8, 2022
(86) PCT No.: PCT/US2022/076092

§ 371 Date: Feb. 29, 2024

Related U.S. Application Data
(60) Provisional application No. 63/241,715, filed on Sep. 8, 2021.

(51) Int. Cl. A61K 31/506; A61K 45/06; A61P 11/00; A61P 37/08

(52) U.S. Cl. CPC A61K 31/506; A61K 45/06; A61P 11/00; A61P 37/08

(57) ABSTRACT

Aspects of the disclosure are directed to methods and compositions for treating or preventing an allergic airway disease in a subject in need thereof. Certain aspects relate to treatment with an effective amount of a composition comprising one or more Dickkopf-1 (Dkk-1) inhibitors and/or one or more additional allergic airway disease therapies. Further aspects relate to methods of reducing airway inflammation, reducing airway hyper-responsiveness, and/or inhibiting an adaptive or innate immune response in the airway of a subject comprising administering to the subject an effective amount of a composition comprising one or more Dkk-1 inhibitors and/or one or more additional allergic airway disease therapies.

### CROSS-REFERENCE TO RELATED APPLICATIONS

This application claims priority to U.S. Provisional Application Ser. No. 63/241,715, filed Sep. 8, 2021, which is incorporated by reference herein in its entirety.

### STATEMENT REGARDING FEDERALLY SPONSORED RESEARCH OR DEVELOPMENT

This invention was made with government support under T32AI053831, R01HL117181, HL140398, and R01AI135803, awarded by the National Institutes of Health, and I01BX004828; awarded by the Veteran's Affairs Office of Research and Development. The government has certain rights in the invention.

### SEQUENCE LISTING

The instant application contains a Sequence Listing which has been submitted in XML format and is hereby incorporated by reference in its entirety. Said XML copy, created on Sep. 6, 2022, is named “BAYM. P0346WO Sequence Listing” and is 2,838 bytes in size.

### BACKGROUND

### I. Field of the Disclosure

Aspects of the disclosure concern at least the fields of cell biology, molecular biology, biochemistry, allergy, mycology, immunology, and medicine.

### II. Background

It has been demonstrated that fungal airway infection (airway mycosis) is an important cause of allergic airway diseases such as asthma. The protein side of the body's coagulation system, namely the clotting protein fibrinogen, is essential to eliciting innate allergic airway inflammation in the context of airway mycosis. However, the mechanisms through which immunity contributes to and may be controlled for treatment of asthmatic immune reactions remain unknown.

The present disclosure satisfies a long-felt need in the art to treat allergic airway diseases by leveraging adaptive and/or innate immune responses.

### SUMMARY

The current disclosure fulfills the need in the art by providing methods and compositions for treating or preventing allergic airway diseases. In some aspects, the allergic airway diseases are resistant to treatment with a corticosteroid. In particular aspects, a subject in need of treatment and/or prevention of an allergic airway disease of any cause is provided an effective amount of one or more Dickkopf-1 (Dkk-1) inhibitors to treat and/or prevent the allergic airway disease. In some cases, the treatment results in effects including but not limited to reducing airway inflammation, reducing airway hyper-responsiveness, and/or inhibiting an adaptive or innate immune response by the subject. In some cases, such a reduction in airway inflammation, reduction in airway hyper-responsiveness, and/or inhibition of an adaptive or innate immune response ameliorates one or more symptoms of an allergic airway disease, including but not limited to inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof.

Severe, treatment refractory asthma and the closely related and often concurrent condition chronic rhinosinusitis (CRS) are causes of serious human morbidity and occasional mortality. Both asthma and CRS are frequently linked to atopy, the predilection to producing IgE antibodies to specific environmental agents. Atopic, or allergic, asthmatics also manifest other signs of systemic inflammation including eosinophilia and local production of predominantly T helper type 2 (Th2) cell cytokines such as interleukin 4 (IL-4), IL-5, and IL-13, but also the Th17 cell cytokine IL-17, all of which are critical for disease expression (Millien et al., 2014; Porter et al., 2011c). A coherent mechanism that explains the coordinated development of the Th2 and Th17 cells that largely define them is unknown.

Asthma and CRS are frequently linked to airway mycosis, the non-invasive growth of fungi along the upper or lower airways, which can be caused by molds such as Aspergillus spp., Penicillium spp., and Alternaria spp (Li et al., 2019). Yeasts such as Candida albicans are furthermore isolated from up to two-thirds of asthma sputum samples and are as capable as molds of inducing asthma-like type 2 lung inflammation and the characteristic exaggerated potential for airway constriction, termed airway hyperresponsiveness, that in aggregate protect the host from potentially lethal dissemination of the fungus (Li et al., 2018; Mak et al., 2013; Porter et al., 2011a; Porter et al., 2009; Porter et al., 2014). The type 17 response is also strongly linked to Candida and other fungal infections and subjects with inborn errors of immune regulation involving Th17 cell responses such as the hyper-IgE syndromes (e.g., Job's Syndrome; DOCK8 deficiency) are afflicted with severe fungal-related diseases such as mucocutaneous candidiasis, asthma and CRS (Boos et al., 2014; Chu et al., 2012; Engelhardt et al., 2015; Eppinger et al., 1999; Huang and Church, 2018; Milner et al., 2008). Although a long-held perception is that C. albicans is not pathogenic when present in the airways of apparently healthy people (Baum, 1960), Candida spp are in fact well-known causes of asthma and the related disorder allergic bronchopulmonary mycosis (Knutsen et al., 2012; Masaki et al., 2017; Sandhu et al., 1979).

The present disclosure concerns key molecular insights into the fundamental and immune basis of the allergic airway diseases and to target causes and symptoms of allergic airway diseases including airway inflammation, airway hyper-responsiveness, and aberrant immune responses in the airway to treat allergic airway diseases. Specific aspects include methods for treating, preventing, or reducing the severity or delaying the onset of one or more allergic airway diseases, methods of reducing airway inflammation, methods of reducing airway hyper-responsiveness, methods of inhibiting an adaptive or innate immune response in the airway of a subject, Dkk-1 inhibitor compositions, allergic airway disease therapies, and/or Dkk-1 inhibitor and allergic airway disease therapy compositions. In some aspects, the allergic airway disease is resistant to treatment with corticosteroids. Thus, the present disclosure provides a unique way to support treatment of allergic airway diseases.

Methods of the present disclosure can include at least 1, 2, 3, 4, 5, or more of the following steps: administering one or more Dkk-1 inhibitors to a subject, administering one or more allergic airway disease therapies to a subject, administering one or more compositions comprising one or more Dkk-1 inhibitors and one or more allergic airway disease therapies to a subject, determining a subject to have a higher risk of developing an allergic airway disease, determining that an allergic airway disease poses a greater risk to the health or life of the subject, determining a subject to have an allergic airway disease, determining a subject to have airway inflammation, determining a subject to have airway hyper-responsiveness, determining a subject to have an adaptive or innate immune response in the airway of the subject, diagnosing a subject with an allergic airway disease, modifying the adaptive or innate immune response of a subject to treat or prevent an allergic airway disease in a subject, identifying a risk of development of an allergic airway disease in a subject, obtaining a biological sample from a subject, and comparing a biological sample from a healthy subject with a biological sample from a subject having an allergic airway disease. It is contemplated that any one or more of these steps may be excluded from certain aspects of the disclosure.

Compositions of the present disclosure can include at least 1, 2, 3, or more of the following components: a Dkk-1 inhibitor or composition thereof, an allergic airway disease therapy or composition thereof, a composition comprising one or more Dkk-1 inhibitors and one or more an allergic airway disease therapies, and one or more pharmaceutical excipients. In specific aspects, the allergic airway disease therapy is an antifungal or composition thereof. In specific aspects, the allergic airway disease therapy is an antibiotic or composition thereof. It is contemplated that any one or more of these components may be excluded from certain aspects of the disclosure.

In some aspects, the Dkk-1 inhibitors inhibit Dkk-1 activity and/or inhibit release of Dkk-1 from platelets. In some aspects, the one or more Dkk-1 inhibitors comprise a small molecule, an antibody, a nucleic acid, or a combination or mixture thereof. The small molecule Dkk-1 inhibitors may comprise WAY 262611, gallocyanine, NCI8642, or functional derivatives thereof. In specific aspects, the Dkk-1 inhibitor is WAY 262611. The antibody Dkk-1 inhibitors may comprise DKN-01 or a functional derivative thereof.

In some aspects, the one or more additional allergic airway disease therapies comprise corticosteroids, leukotriene modifiers, bronchodilators, antifungals, biologics, allergy shots, antihistamines, decongestants, cromolyn, or combinations thereof. In specific aspects, the corticosteroids comprise fluticasone, dexamethasone, budesonide, mometasone, beclomethasone, and/or ciclesonide. In specific aspects, the leukotriene modifiers comprise montelukast, zafirlukast, and/or zileuton. In specific aspects, the bronchodilators comprise theophylline, albuterol, levalbuterol, ipratropium, aclidinium, arformoterol, formoterol, indacaterol, tiotropium, salmeterol, glycopyrrolate, olodaterol, vilanterol, and/or umeclidinium. In specific aspects, the antifungals comprise amphotericin B, azithromycin, terbinafine, voriconazole, itraconazole, fluconazole, isavuconazole, posaconazole, ketoconazole, micafungin, ibrexafungerp, and/or caspofungin. In specific aspects, the biologics comprise omalizumab, mepolizumab, benralizumab, dupilumab, and/or reslizumab. In specific aspects, the antihistamines comprise azelastine, brompheniramine, cetirizine, chlorpheniramine, desloratadine, diphenhydramine, doxylamine, fexofenadine, hydroxyzine, ketotifen, loratadine, levocetirizine, and/or olaptadine. In specific aspects, the decongestants comprise levmetamfetamine, naphazoline, pseudoephedrine, phenylephrine, propylhexedrine, oxymetazoline, and/or xylometazoline.

Disclosed herein, in some aspects, are methods of treating or preventing an allergic airway disease in a subject in need thereof comprising administering to the subject an effective amount of a composition comprising one or more Dickkopf-1 (Dkk-1) inhibitors. In some aspects, the allergic airway disease comprises chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof. In particular cases, the allergic airway disease is asthma. In some cases, the allergic airway disease is caused at least in part by one or more irritants and/or immune activators comprising fungal infection, mold or mildew, hair or dander, dust, pollen, smoke, exercise, stress, perfume or other strong odors, air pollution comprising ozone, particulates, nitrogen dioxides, or sulfate aerosols, and/or changes in the weather. In specific cases, the allergic airway disease is caused at least in part by fungal infection. In specific aspects, disclosed are methods of treating or preventing asthma in a subject in need thereof, wherein the asthma is caused at least in part by a fungal infection, the method comprising administering to the subject an effective amount of a composition comprising one or more Dickkopf-1 (Dkk-1) inhibitors.

In some aspects, the fungal infection comprises Aspergillus spp., Penicillium spp., Alternaria spp., Penicillium spp., Curvularia spp., Bipolaris, Mucor spp., Rhizopus spp., Pneumocystis spp., Aureobasidia spp., Cladosporium spp., Cochliobus spp., Paecilomyces spp., Trichoderma spp., Trichosporon spp., Malassezia spp., and/or Candida spp. fungi. In certain aspects, the fungal infection comprises Aspergillus spp. In specific aspects, the Aspergillus fungi comprise Aspergillus flavus, Aspergillus fumigatus, Aspergillus nidulans, Aspergillus oryzae, Aspergillus sydowii, Aspergillus versicolor, Aspergillus wentii, and/or Aspergillus niger. In certain aspects, the fungal infection comprises Candida spp. In specific aspects, wherein the Candida fungi comprise Candida albicans, Candida tropicalis, Candida glabrata, Candida auris, Candida lusitaniae, Candida parapsilosis, Candida krusei, Candida dubliniensis, and/or Candida guilliermondii.

The allergic airway disease may be characterized by one or more symptoms comprising inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof.

In some aspects, the allergic airway disease, e.g., asthma, is resistant to treatment with corticosteroids. In some aspects, treating or preventing the allergic airway disease comprises reducing airway inflammation. In some aspects, treating or preventing the allergic airway disease comprises reducing airway hyper-responsiveness. In some aspects, treating or preventing the allergic airway disease comprises inhibiting an adaptive or innate immune response by the subject. In some aspects, inhibiting the adaptive or innate immune response by the subject comprises inhibiting cytokine secretion and/or inhibiting recruitment or activity of inflammatory cells and/or T helper effector cells. In specific aspects, the cytokines comprise interleukin-2, interleukin-4, interleukin-5, interleukin-6, interleukin-9, interleukin-10, interleukin-12, interleukin-13, interleukin-17A, interleukin-17B, interleukin-17C, interleukin-17D, interleukin-17E, interleukin-17F, interleukin-22, interleukin-33, tumor necrosis factor, thymic stromal lymphopoietin, ciliary neurotrophic factor, or interleukin-1β. In specific aspects, the inflammatory cells comprise granulocytes and/or macrophages. In specific aspects, the T helper effector cells comprise T helper type 2 (Th2) cells and/or T helper type 17 (Th17) cells.

Disclosed herein, in some aspects, are methods of reducing airway inflammation, reducing airway hyper-responsiveness, and/or inhibiting an adaptive or innate immune response in the airway of a subject comprising administering to the subject an effective amount of a composition comprising one or more Dickkopf-1 (Dkk-1) inhibitors. In some aspects, the airway inflammation, airway hyper-responsiveness, and/or adaptive or innate immune response in the airway of a subject are caused by an allergic airway disease, and in specific aspects, the allergic airway disease is asthma. In some aspects, the inflammation, hyper-responsiveness, and/or adaptive or innate immune response is caused at least in part by one or more irritants and/or immune activators comprising fungal infection, mold or mildew, hair or dander, dust, pollen, smoke, exercise, stress, perfume or other strong odors, air pollution comprising ozone, particulates, nitrogen dioxides, or sulfate aerosols, and/or changes in the weather. The one or more irritants and/or immune activators may cause allergic airway disease, which in specific aspects, may be asthma. In specific aspects, the inflammation, hyper-responsiveness, and/or adaptive or innate immune response is caused at least in part by fungal infection. The fungal infection may cause allergic airway disease, which in specific aspects, may be asthma. In some aspects, the fungal infection comprises Aspergillus spp., Penicillium spp., Alternaria spp., Penicillium spp., Curvularia spp., Bipolaris, Mucor spp., Rhizopus spp., Pneumocystis spp., Aureobasidia spp., Cladosporium spp., Cochliobus spp., Paecilomyces spp., Trichoderma spp., Trichosporon spp., Malassezia spp., and/or Candida spp. fungi. In certain aspects, the fungal infection comprises Aspergillus spp. In specific aspects, the Aspergillus fungi comprise Aspergillus flavus, Aspergillus fumigatus, Aspergillus nidulans, Aspergillus oryzae, Aspergillus sydowii, Aspergillus versicolor, Aspergillus wentii, and/or Aspergillus niger. In certain aspects, the fungal infection comprises Candida spp. In specific aspects, wherein the Candida fungi comprise Candida albicans, Candida tropicalis, Candida glabrata, Candida auris, Candida lusitaniae, Candida parapsilosis, Candida krusei, Candida dubliniensis, and/or Candida guilliermondii. In some aspects, the inflammation, hyper-responsiveness, and/or adaptive or innate immune response are symptoms of an allergic airway disease disclosed herein. In specific aspects, the inflammation, hyper-responsiveness, and/or adaptive or innate immune response are symptoms of asthma, as disclosed herein.

In some aspects, the allergic airway disease, e.g., asthma, is resistant to treatment with corticosteroids. In some aspects, inhibiting the adaptive or innate immune response in the airway of the subject comprises inhibiting cytokine secretion and/or inhibiting recruitment or activity of inflammatory cells and/or T helper effector cells. In specific aspects, the cytokines comprise interleukin-2, interleukin-4, interleukin-5, interleukin-6, interleukin-9, interleukin-10, interleukin-12, interleukin-13, interleukin-17A, interleukin-17B, interleukin-17C, interleukin-17D, interleukin-17E, interleukin-17F, interleukin-22, interleukin-33, tumor necrosis factor, thymic stromal lymphopoietin, ciliary neurotrophic factor, or interleukin-1l. In specific aspects, the inflammatory cells comprise granulocytes and/or macrophages. In specific aspects, the T helper effector cells comprise T helper type 2 (Th2) cells and/or T helper type 17 (Th17) cells.

In some aspects of the methods, the subject is provided an effective amount one or more additional therapies for allergic airway disease disclosed herein. In specific aspects, the one or more Dkk-1 inhibitors and one or more additional therapies are administered in the same composition. In specific aspects, the one or more Dkk-1 inhibitors and one or more additional therapies are administered in different compositions. In some aspects, the composition further comprises one or more pharmaceutically acceptable excipients.

In some aspects, the composition is administered intranasally, subcutaneously, intravenously, by aerosol, and/or orally. In some aspects of the methods, the composition is administered once or multiple times. In specific aspects, the composition is administered to the individual multiple times, the duration between administrations is within 1-24 hours, 1-7 days, 1-4 weeks, or 1-12 months.

In some aspects, when providing the composition to the subject according to the methods disclosed herein, the subject had or was at risk of having chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof.

In some aspects, the methods further comprise the step of identifying that the subject had or was at risk of having chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof.

Disclosed herein, in some aspects, is a composition comprising: (a) one or more Dkk-1 inhibitors disclosed herein; and (b) one or more allergic airway disease therapies disclosed herein. In specific aspects, the one or more Dkk-1 inhibitors and the one or more allergic airway disease therapies are in different formulations. In specific aspects, the one or more Dkk-1 inhibitors and the one or more allergic airway disease therapies are in the same formulation. In some aspects, the composition further comprises one or more pharmaceutically acceptable excipients.

The foregoing has outlined rather broadly the features and technical advantages of the present disclosure in order that the detailed description that follows may be better understood. Additional features and advantages will be described hereinafter which form the subject of the claims herein.

It should be appreciated by those skilled in the art that the conception and specific aspects disclosed may be readily utilized as a basis for modifying or designing other structures for carrying out the same purposes of the present designs. Use of the one or more compositions may be employed based on any of the methods described herein. Other aspects are discussed throughout this application. Any aspect discussed with respect to one aspect of the disclosure applies to other aspects of the disclosure as well and vice versa. For example, any step in a method described herein can apply to any other method. Moreover, any method described herein may have an exclusion of any step or combination of steps. The aspects in the Example section are understood to be aspects that are applicable to all aspects of the technology described herein. It is contemplated that any aspect discussed in this specification can be implemented with respect to any method or composition of the disclosure, and vice versa. Furthermore, compositions of the disclosure can be used to achieve methods of the disclosure. It should also be realized by those skilled in the art that such equivalent constructions do not depart from the spirit and scope as set forth in the appended claims.

The novel features which are believed to be characteristic of the designs disclosed herein, both as to the organization and method of operation, together with further objects and advantages will be better understood from the following description when considered in connection with the accompanying figures. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIGS. 1A-1H. Candidalysin is necessary for the induction of allergic airway disease in mice. FIG. 1A. C57BL/6 mice were challenged intranasally with 10⁵ viable cells of wildtype parental strain or ece1Δ/Δ C. albicans as indicated in the timeline. FIG. 1B. Respiratory system resistance (R_{R}S) was assessed after intravenous injection of increasing doses of acetylcholine (Ach). FIG. 1C. Quantitation of cells from bronchoalveolar lavage fluid samples (mac: macrophages; eos: eosinophils; neu: neutrophils; lym: lymphocytes). FIG. 1D. Cytokines quantitated by ELISA from deaggregated lung supernatants. FIGS. 1E-1F. T cells from lungs analyzed by flow cytometry. FIG. 1E. Representative flow plot of TH1 (T-bet positive), Th2 (GATA3 positive) and Th17 (RORγt positive) cells from lungs after challenge. FIG. 1F. Aggregate T cell data expressed as percentages and absolute cell numbers. FIG. 1G. C. albicans colony forming units (CFU) cultured from lungs. FIG. 1H. Hematoxylin and eosin (H&E) and periodic acid-Schiff (PAS) staining of 5 μm lung sections from mice challenged under the indicated conditions. (n≥4, mean±S.E.M, n.s.: not significant, *p<0.05, **p<0.01, ***p<0.001, ****p<0.0001, using two-tailed Student's t-test (FIG. 1F) or one-way ANOVA followed by Tukey's test (FIGS. 1A-1D, 1G) for multiple comparison. Magnification: 40× and 200×. Bars: 500 or 50 m, respectively). Data are representative of three independent experiments. See also FIG. 7 and FIG. 8.

FIGS. 2A-2N. Dkk-1 is secreted by mouse and human platelets in response to candidalysin and is required for robust allergic airway disease and C. albicans clearance from lung. FIG. 2A. Plasma Dkk-1 concentrations from patients with asthma and CRS as compared non-allergic healthy controls. Dkk-1 concentrations quantitated from plasma of mice challenged intranasally with (FIG. 2B) wildtype parental strain or ece1Δ/Δ C. albicans or (FIG. 2C) candidalysin (CL) or scrambled control (SC). FIG. 2D. Dkk-1 was quantitated from platelets of mice challenged intranasally with wildtype or ece1Δ/Δ C. albicans. Human platelets in plasma were incubated with either C. albicans (FIG. 2E) or CL/SC (FIG. 2F) after which secreted Dkk-1 was quantitated. FIG. 2G. C57BL/6 mice were challenged intranasally with C. albicans (C.a) and intraperitoneally with Dkk-1 inhibitor (WAY262611) as shown. FIG. 2H. Respiratory system resistance (R_{RS}) was quantitated as in FIG. 1. FIG. 2I. Quantitation of cells from the bronchoalveolar lavage fluid (mac: macrophages; eos: eosinophils; neu: neutrophils; lym: lymphocytes). FIGS. 2J-2K. Cytokines assessed by ELISA from lung homogenate supernatants.

FIGS. 2L-2M. T cell quantitation from lungs as determined by flow cytometry. FIG. 2L. Gating strategy for quantitation of T_{H}1, Th2 and Th17 cells from lungs. FIG. 2M. Quantitation of T cells assessed as percentages and absolute cell counts. FIG. 2N. Lung fungal burdens. (n≥4, mean±S.E.M, n.s.: not significant, *p<0.05, **p<0.01, ***p<0.001, ****p<0.0001, using one-way ANOVA followed by Tukey's test for multiple comparisons). Data are representative of two independent experiments. See also FIG. 9.

FIGS. 3A-3H. Recombinant Dkk-1 enhances allergic airway disease in ece1Δ/Δ C. albicans-challenged mice. FIG. 3A. Wildtype mice were challenged intranasally with ece1Δ/Δ C. albicans (C.a) and intraperitoneally with recombinant mouse Dkk-1 as shown. FIG. 3B. Respiratory system resistance (R_{R}S) was assessed by increasing intravenous acetylcholine challenge. FIG. 3C. Quantitation of cells from bronchoalveolar lavage fluid. FIGS. 3D-3E. Cytokines assessed by ELISA from lung homogenate supernatants. FIGS. 3F-3G T cell quantification from lungs as assessed by flow cytometry. FIG. 3F. Representative flow cytometry plot of T_{H}1, Th2 and Th17 cells from lungs after challenge. FIG. 3G. Quantitation of T cells as expressed as percentages and absolute cell numbers. FIG. 3H. C. albicans CFU retrieved from whole lung (n≥4, mean±S.E.M, n.s.: not significant, *p<0.05, **p<0.01, ***p<0.001, ****p<0.0001, using two-tailed Student's t-test (FIG. 3G) or one-way ANOVA followed by Tukey's test (FIGS. 3A-3E) for multiple comparison). Data are representative of two independent experiments.

FIGS. 4A-4E. Candidalysin primes human platelets to release Dkk-1 via GP1bα. FIG. 4A. Human platelets were incubated with PBS or candidalysin (CL) at 10 μM and with blocking reagents to the indicated platelet receptors as indicated after which secreted Dkk-1 was quantitated. Schematic diagrams and aggregate data depicting in vitro assays in which the dose-dependent binding of either plate-bound candidalysin or scrambled control peptide (SC) (FIG. 4B) or GP1bα (FIG. 4C) to the other reagent was determined colorimetrically (OD: optical density). FIG. 4D. Schematic diagram and aggregate data depicting in vitro binding assays with GP1bα blocking reagents (anti-GP1bα antibody; VWF A1A2A3 tridomain) in which the dose-dependent inhibition of binding of candidalysin is colorimetrically quantitated against plates coated with GP1bα. FIG. 4E. Pull down assay of GP1bα from human platelet lysates using biotinylated candidalysin or SC as bait. (n≥4, mean±S.E.M, *p<0.05, **p<0.01, ***p<0.001 and ****p<0.0001 using Kurskal-Wallis test or one-way ANOVA followed by Tukey's test for multiple comparison. Illustrative figures generated at biorenders.com). Data are representative of two independent experiments.

FIGS. 5A-5E. Candidalysin directly binds to human platelets via GP1bα. Flow cytometric analysis of human platelets incubated with AF647-conjugated CL (10 μM) with or without anti-GP1bα antibody (FIG. 5A) or VWF A1A2A3 tridomain (FIG. 5B). FIG. 5C. Flow cytometric analysis of P-selectin (CD62P) on human platelets after incubation with CL (10 μM) without or with anti-GP1bα antibody. Representative histograms, percentage quantification, and median fluorescence intensity (MFI) data are shown. Human platelets were washed and then resuspended in PBS with blocking anti-GP1bα antibody or isotype control antibody prior to addition of candidalysin (CL; 10 μM) after which platelet counts (FIG. 5D) and Dkk-1 concentrations (FIG. 5E) from supernatants were determined. (n=4, mean±S.E.M, ***p<0.001 and ****p<0.0001 using one-way ANOVA followed by Tukey's test for multiple comparison). Data are representative of two independent experiments. See also FIG. 10.

FIGS. 6A-6G. Thrombocytopenic mice rapidly succumb to C. albicans airway challenge. FIG. 6A. Total platelet count in whole blood from mice 2 h after platelet depletion with anti-GP1bα antibody. FIG. 6B. Survival curves (hours) of platelet-depleted mice challenged once intranasally with PBS or 10⁵ wildtype parental strain or ece1Δ/Δ C. albicans. FIG. 6C. Survival curve of platelet-depleted mice challenged intranasally with 16 μmol candidalysin or PBS. FIGS. 6D-6E. Bronchoalveolar lavage fluid and whole lungs were collected 4 h after platelet-depleted mice were challenged intranasally with wildtype or ece1Δ/Δ C. albicans. FIG. 6D. Gross appearance of lungs. FIG. 6E. Microscopic appearance of lungs (H&E staining). FIG. 6F Quantitation of hemoglobin from BALF. FIG. 6G. Representative microCT-based imaging of platelet sufficient and depleted mice challenged with either wildtype or ece1Δ/Δ C. albicans as indicated. Bar graphs depict lung density as measured in Hounsfield units and aerated lung volume H: heart; L: lung; #: areas of abnormal alveolar filling (n≥4, mean±S.E.M, *p<0.05, **p<0.01, ***p<0.001 and ****p<0.0001 using one-way ANOVA followed by Tukey's test for multiple comparison, or Log-rank test for survival curves.) Data are representative of two independent experiments. See also FIG. 11.

FIGS. 7A-7F. Proteinase from C. albicans is not required for allergic airway disease. FIG. 7A. Wildtype (WT) or TLR4^{-/-} C57BL/6 mice were challenged intranasally with 10⁵ viable cells of WT (Parental control), secreted aspartic proteinase 1, 2, 3 triple deficient (SAP1-3Δ/Δ or SAP4-6Δ/Δ) C. albicans every two days over 17 days. FIG. 7B. Respiratory system resistance (RRS) was assessed in response to increasing intravenous acetylcholine (Ach) challenges. FIG. 7C. Quantitation of cells from bronchoalveolar lavage fluid (mac: macrophages; eos: eosinophils; neu: neutrophils; lym: lymphocytes). FIG. 7D. Cytokines quantitated by ELISA from deaggregated lung. SDS-PAGE gel electrophoresis assay showing degradation of fibrinogen by purified secreted aspartic proteinases (Saps) from C. albicans or the proteinase from Aspergillus melleus (PAM) over the indicated times (FIG. 7E), or by recombinant Saps individually or combined for 6 hours (FIG. 7F). (n≥4, mean±S.E.M, n.s.: not significant, *p<0.05, **p<0.01, using one-way ANOVA followed by Tukey's test for multiple comparisons). Data are representative of three independent experiments.

FIGS. 8A-8E. Candidalysin induces allergic airway disease. FIG. 8A. Protocol for administering synthetic candidalysin intranasally to anesthetized wildtype mice. Respiratory system resistance (RRS) (FIG. 8B), BALF cells (FIG. 8C), and lung cytokines (FIGS. 8D-8E) were quantitated as in FIG. 1. (n≥4, mean±S.E.M, n.s.: not significant, *p<0.05, **p<0.01, using one-way ANOVA followed by Tukey's test for multiple comparisons). Data are representative of three independent experiments.

FIGS. 9A-9E. Platelet and pulmonary megakaryocytes reacts to Candidalysin. FIGS. 9A-9C. Anesthetized wildtype C57BL/6 mice were challenged intranasally 8 times over 17 days with WT (parenta) or ece1Δ/Δ C. albicans. Platelet count from mice post challenge. Lungs were removed 24 hours after the final challenge (FIG. 9A) and quantified for Dkk-1^{high} megakaryocytes (as indicated by the gating box) and Dkk-1 MFI (FIG. 9B). FIG. 9C. Total megakaryocytes were quantified by flow cytometry. FIG. 9D. Mice are challenge intranasally with 16 μmol of candidalysin (CL) or scrambled control (SC) and platelets were isolated from left and right ventricle 2 hours post challenge. Dkk-1 quantified from platelets from the left and right ventricle. FIG. 9E. EOMA cells were treated with candidalysin at 10 and 20 μM. Dkk-1 was quantified in the supernatant Illustrative figures generated at biorenders.com (n≥4, mean±S.E.M, n.s.: not significant, *p<0.05, **p<0.01, ***p<0.001, ****p<0.0001 using one-way ANOVA followed by Tukey's test for multiple comparisons).

FIGS. 10A-10G. Candidalysin binds to and activates human platelets. Human platelets were treated with 10 μM biotinylated candidalysin (Bio-CL) or scrambled control (Bio-SC), followed by Streptavidin-Alexafluor 647 after which flow cytometry was used to determine binding as AF647% positive cells (FIG. 10A) and median fluorescence intensity (MFI) (FIG. 10B). Human platelets were prepared with candidalysin (CL) (10 or 20 μM), scrambled control (SC) or PBS after which flow cytometry was used to determine the change in expression of the activation marker P-selectin (CD62P) expressed as % positive cells (FIG. 10C) and mean fluorescence intensity (MFI) (FIG. 10D). FIG. 10E. Schematic diagrams and aggregate data depicting in vitro assays in which the dose-dependent binding of plate-bound GP1bα or GPIIb/IIIa to candidalysin was determined colorimetrically. FIG. 10F. Human platelets were prepared with 20 μM candidalysin (CL) or PBS after which flow cytometry was used to determine the change in expression of the activation marker GPIIb/IIIa expressed as % positive cells and mean fluorescence intensity (MFI). FIG. 10G. Percentage aggregation for platelets in response to the indicated doses of collagen or candidalysin. (n≥4, mean±S.E.M, n.s.: not significant, *p<0.05, **p<0.01, ***p<0.001, ****p<0.0001 using one-way ANOVA followed by Tukey's test for multiple comparisons). Data are representative of three independent experiments.

FIGS. 11A-11H. Lung histology of mice after platelet depletion or C. albicans challenge, and candidalysin failed to induce airway hyper-reactivity to platelet depleted mice. FIG. 11A. GMS staining on lung sections at 200× from wildtype platelet-depleted mice after C. albicans challenge. Reference bar: 500 and 50 μM, respectively. Wildtype, platelet-sufficient mice were challenged once with C. albicans intranasally (FIG. 11B) or platelet depleted without C. albicans challenge (FIG. 11C). Lungs were removed 4 hours after either challenge, and H&E staining on 5 μm lung sections at 40× (inset at 200×) was performed. Reference bar: 500 and 50 μM, respectively. FIG. 11D. Wildtype mice were challenged intranasally with 8 μM of recombinant candidalysin intranasally, with or without platelet depleting antibody intraperitoneally every two days over 17 days. FIG. 11E. Respiratory system resistance (RRS) was assessed in response to increasing intravenous acetylcholine (Ach) challenges. FIG. 11F. Quantitation of cells from bronchoalveolar lavage fluid (mac: macrophages; eos: eosinophils; neu: neutrophils; lym: lymphocytes). FIG. 11G. Cytokines quantitated by ELISA from deaggregated lung. (FIG. 11H) Dkk-1 quantitated by ELISA from mouse plasma. (n=3, mean±S.E.M, n.s.: not significant, *p<0.05, **p<0.01, ***p<0.001, using one-way ANOVA followed by Tukey's test for multiple comparisons). Data are representative of two independent experiments.

FIGS. 12A-12E. Gating strategy for TH cells (FIG. 12A) platelets (FIG. 12B), and megakaryocytes (FIG. 12C). Uncropped membranes from western blot of GP1bα pulldown (FIG. 12D) and cleavage product (FIG. 12E) from Saps or PAM.

FIGS. 13A-13B show Dkk-1 release as measured by ELISA from platelets stimulated with lipopolysaccharide (LPS), thrombin, prothrombin (ProThrom), proteinase of Aspergillus melleus (PAM), or candidalysin (C. Lysin) at 4° C. (FIG. 13A) or 37° C. (FIG. 13B).

FIGS. 14A-14B. Severe eosinophilic and neutrophilic steroid-resistant allergic airway disease (AAD) models. FIG. 14A shows an experimental timeline for assessing the effect of daily steroid (fluticasone propionate; FP) or vehicle (liposome: dilauroylphosphatidylcholine: DLPC) treatment on airway hyperresponsiveness (AHR) measured as a function of respiratory system resistance (R_{RS}(cmH₂O·s·ml⁻¹)) in C57BL/6 mice challenged intranasally (q.o.d.) with 4×10⁵ A. niger (AN) conidia ±30 ng lipopolysaccharide (LPS) (FIG. 14B).

FIGS. 15A-15E show experimental timelines (FIG. 15A) for assessing the effect of steroid treatment (dexamethasone; Dex) on airway hyperresponsiveness (AHR) in an eosinophilic steroid-resistant allergic airway disease (AAD) model. C57BL/6 mice were treated over 28 days as follows: PBS (control) daily (PBS→PBS); intranasal challenge (q.o.d.) with 4×10⁵ A. niger (AN) conidia every other day (AN→AN; FIG. 15A, top); intranasal challenge (q.o.d.) with 4×10⁵ A. niger (AN) conidia every other day+Dex daily (AN/Dex→AN/Dex; FIG. 15A, middle); intranasal challenge (q.o.d.) with 4×10⁵ A. niger (AN) conidia every other day+Dex daily on days 14-28 (AN→AN/Dex; FIG. 15A, bottom). Effect was measured as a function of respiratory system resistance (R_{RS}(cmH₂O·s·ml⁻¹)) (FIG. 15B), immune cell response (FIG. 15C), cytokine release (FIG. 15D), and Th17 (RORγt positive) cell response (FIG. 15E) in C57BL/6 mice.

FIG. 16 shows that treatment with a Dkk-1 antagonist abrogates airway hyperresponsiveness (AHR) in fungal experimental asthma due to Aspergillus niger (AN).

### DETAILED DESCRIPTION

### I. Examples of Definitions

The use of the word “a” or “an” when used in conjunction with the term “comprising” may mean “one,” but it is also consistent with the meaning of “one or more,” “at least one,” and “one or more than one.” As used in the specification and claims, the singular form “a”, “an”, and “the” include plural references unless the context clearly dictates otherwise.

The compositions and methods for their use can “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

The word “or” or the phrase “and/or” means “and” or “or”. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect.

Throughout this application, the term “about” is used according to its plain and ordinary meaning in the area of cell and molecular biology to indicate that a value includes the inherent variation or standard deviation of error for the measurement or quantitation method being employed to determine the value.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

“Functional derivative,” as used herein, refers to a derivative compound having a distinct structure while at the same time retaining substantially the same activity as the parent compound. Functional derivatives of Dkk-1 inhibitors are thus defined herein as those compounds that retain activity for inhibiting Dkk-1; reducing inflammation, reducing airway hyper-responsiveness, and/or inhibiting an adaptive or innate immune response in the airway or a subject; and/or treating one or more symptoms of allergic airway disease including but not limited to inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof. Functional derivatives of allergic airway disease therapies as thus defined herein as those compounds that retain activity for reducing inflammation, reducing airway hyper-responsiveness, and/or inhibiting an adaptive or innate immune response in the airway or a subject; and/or treating one or more symptoms of allergic airway disease including but not limited to inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof. As an example, a functional derivative of a compound that is a bronchodilator would have activity for dilating bronchial tubes.

“Individual, “subject,” and “patient,” are used interchangeably herein and generally refers to an individual in need of treatment. The subject can be any animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more cancers. The subject may be undergoing or having undergone cancer treatment. The “subject” or “individual,” as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants and includes in utero individuals. The individual may be of any gender or race or ethnicity.

### II. Aspects of the Disclosure

The present disclosure is based, at least in part, on the surprising discovery that platelets play an important role in promoting allergic inflammation by driving both Th2 and Th17 development upon release of Dkk-1. As disclosed herein, fungal airway infection (airway mycosis) and other irritants and/or activators of immune response by a subject are important causes of allergic airway diseases such as asthma, but the mechanisms by which irritants and/or immune activators like fungi trigger immune responses, such as asthmatic reactions, are poorly understood.

Secreted proteinases from molds elicit robust Th2 cell responses and experimental allergic airway disease. In part, proteinases cleave fibrinogen, immunostimulatory fragments of which signal through TLR4 to elicit allergic airway disease. While important, this pathway fails to activate Th2 and Th17 cells and instead acts strictly on innate immune cells such as airway epithelial cells, macrophages, and innate lymphoid cells (ILC) (Landers et al., 2019b; Millien et al., 2013). Despite the many proteinases secreted by C. albicans (Naglik et al., 2003), they play at most a minor role in inducing robust Th2 and Th17 cell-driven allergic airway disease, in part presumably because these aspartic-class proteinases fail to cleave fibrinogen into immunologically active fragments.

In the case of Aspergillus niger, in some aspects, the main virulence factor is A. niger proteinases, which activate two pathways to promote expression of allergic airway disease: first, an innate immune pathway in which the proteinases cleave fibrinogen into fibrinogen cleavage products that activate the transcription factor STAT6 via TLR4, which may ultimately drive AHR and other aspects of the asthma phenotype; and second, an adaptive immune pathway in which the proteinases may activate platelets (e.g., by converting prothrombin to thrombin; thrombin then activates platelets via one or more platelet activated receptor (PAR)) to release Dkk-1 that drives the Th2 and Th17 responses. Similar to C. albicans, in some aspects, the Dkk-1 that is released drives Th2 and Th17 responses for a robust asthma phenotype.

In this disclosure, it is shown that wildtype and mutant Candida albicans elicit Th2 and Th17 cell-dependent allergic airway disease through the peptide toxin candidalysin. Candidalysin activated platelets through the Von Willebrand factor (VWF) receptor GP1bα to release the Wnt antagonist Dickkopf-1 (Dkk-1) to drive Th2 and Th17 cell responses that correlated with reduced lung fungal burdens. Platelets simultaneously precluded lethal pulmonary hemorrhage resulting from fungal lung invasion. Thus, strong convergent evidence is provided herein that, in addition to the protein side of the coagulation system, i.e., fibrinogen, the other major (cellular) component of the coagulation system, platelets, can also promote allergic inflammation and drive Th2 and Th17 development by releasing Dkk-1 under the control of fungal signals. This suggests that, in some aspects, Dkk-1 inhibitors or inhibitors that block platelet secretion of Dkk-1 without compromising the essential clotting/thrombosis activity of platelets might be useful as anti-inflammatory agents in severe asthma and related allergic disorders.

Accordingly, in some aspects, disclosed are methods and compositions for treating or preventing allergic airway diseases. In particular aspects, a subject in need of treatment and/or prevention of an allergic airway disease of any cause is provided an effective amount of one or more Dickkopf-1 (Dkk-1) inhibitors to treat and/or prevent the allergic airway disease. The allergic airway disease may be resistant to treatment with corticosteroids. In some cases, the treatment results in effects including but not limited to reducing airway inflammation, reducing airway hyper-responsiveness, and/or inhibiting an adaptive or innate immune response by the subject. In some cases, such a reduction in airway inflammation, reduction in airway hyper-responsiveness, and/or inhibition of an adaptive or innate immune response ameliorates one or more symptoms of an allergic airway disease, including but not limited to inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof.

The results encompassed herein demonstrate that novel approaches can treat or prevent allergic airway diseases. Major forms of allergic airway diseases like asthma, including multiple distinct endotypes, are characterized by the presence of Th2 and Th17 cells that are both important for disease expression and, in the context of airway mycosis, for both fungal eradication and prevention of invasion and fungal dissemination. As disclosed herein, C. albicans can elicit Th2 and Th17 responses in experimental allergic airway disease through the secreted peptide toxin candidalysin. Platelets respond to candidalysin through the VWF receptor GP1bα to specifically release Dkk-1, a Wnt pathway antagonist peptide that coordinates the generation of Th2 and Th17 cells in this context. Candidalysin and Dkk-1 were involved in both expression of allergic airway disease in mice and optimal control of lung fungal burdens, further linking the importance of allergic inflammation to the control of airway mycosis. As only mice with circulating platelets were capable of secreting Dkk-1 into the plasma and responding to candidalysin challenge with Th2 and Th17 cell responses and expressing allergic airway disease, these findings provide substantial evidence that, in some aspects, platelets can be an important source of Dkk-1 during C. albicans airway mycosis. Thus, in particular aspects, Dkk-1 may be an important determinant of human allergic airway disease, and the present disclosure provides methods and compositions utilizing Dkk-1 inhibitors for treatment of allergic airway disease, including, in some aspects, allergic airway disease that is resistant to treatment with corticosteroids.

### III. Platelets & Immunity

In the present disclosure, platelets secrete Dkk-1, which activates immune responses in an individual. In some aspects, release of Dkk-1 from platelets is selectively inhibited using one or more Dkk-1 inhibitors. In some aspects, the methods and compositions encompassed herein provide allergic airway disease therapy that blocks platelet secretion of Dkk-1 without compromising the essential clotting/thrombosis activity of platelets.

Rather than being initially detected by a canonical immune cell, megakaryocytes and platelets are first responders to C. albicans airway mycosis. Platelets, also called thrombocytes, are a component of blood whose function (along with the coagulation factors) is to react to bleeding from blood vessel injury by clumping, thereby initiating a blood clot. Platelets have no cell nucleus; they are fragments of cytoplasm that are derived from the megakaryocytes of the bone marrow, which then enter the circulation.

As described herein, platelets can participate in both innate and adaptive intravascular immune responses. Platelets are rapidly deployed to sites of injury or infection, and potentially modulate inflammatory processes by interacting with leukocytes and by secreting cytokines, chemokines and other inflammatory mediators. Platelets also secrete platelet-derived growth factor (PDGF). Platelets modulate neutrophils by forming platelet-leukocyte aggregates (PLAs). These formations induce upregulated production of αmβ2 (Mac-1) integrin in neutrophils. Interaction with PLAs also induce degranulation and increased phagocytosis in neutrophils. Platelets are also the largest source of soluble CD40L which induces production of reactive oxygen species (ROS) and upregulate expression of adhesion molecules, such as E-selectin, ICAM-1 and VCAM-1, in neutrophils, activates macrophages and activates cytotoxic response in T and B lymphocytes.

With respect to adaptive immunity, platelets are capable of interacting with antibodies. They are able to specifically bind IgG through a FcγRIIA receptor for the constant fragment (Fc) of IgG. When activated and bound to IgG, the platelets can subsequently release reactive oxygen species (ROS), antimicrobial peptides, defensins, kinocidins, and proteases, killing microbes directly. Platelets can also secrete proinflammatory and procoagulant mediators such as inorganic polyphosphates or platelet factor 4 (PF4), connecting innate and adaptive immune responses.

Another major function of platelets is to contribute to hemostasis: the process of stopping bleeding at the site of interrupted endothelium. They gather at the site and, unless the interruption is physically too large, they plug the opening in the endothelium. First, platelets adhere to substances outside the interrupted endothelium. Second, they change shape, activate receptors, and secrete chemical messengers. Third, they connect to each other, or aggregate, through receptor bridges. Formation of this platelet plug (primary hemostasis) is associated with activation of the coagulation cascade, with resultant fibrin deposition and linking (secondary hemostasis). The platelet cell membrane also has receptors for collagen. Following the rupture of the blood vessel wall, the platelets are exposed and they adhere to the collagen in the surrounding connective tissue.

As described herein, candidalysin is recognized immunologically by platelets through a cognate interaction with GP1bα and not other platelet receptors such as GPIIb/IIIa. The only other known ligand for GP1bα is VWF, an exceptionally large, multimeric protein that is produced by vascular endothelial cells and platelets. Under conditions of shear stress especially in the context of damaged endothelium in which both collagen and VWF become exposed to circulating platelets, the VWF-GP1bα interaction activates platelets to adhere and aggregate, thereby providing an essential hemostatic function. In contrast, ligation of GP1bα with candidalysin failed to elicit thrombosis, but rather activated platelets to express CD62P and secrete Dkk-1 acutely; with more prolonged exposure, candidalysin-activated platelets underwent autolysis rather than aggregation.

During C. albicans airway mycosis, but also during invasive aspergillosis (Tischler et al., 2020), platelets can protect the host from the potentially lethal effects of microhemorrhages that likely result from fungal invasion of the airway microvasculature. C. albicans-related thrombosis therefore may not be the result of candidalysin directly signaling through platelets, but rather may be by the effect of VWF (and collagen) exposed through fungal invasion and tissue disruption. Thrombin (coagulation factor II), which may be activated in this context, may also independently induce thrombosis by signaling through proteinase-activated receptors (PARs) (Sambrano et al., 2001). Without wishing to be bound by theory, these observations raise the possibility that GP1bα may signal differentially according to the ligand encountered, eliciting either a predominant secretory or autolytic response through exogenous candidalysin or a thrombotic response through endogenous VWF. These diverse platelet functions operate simultaneously during airway mycosis, and GP1bα ligands may be developed that differentially activate these functions to achieve distinct therapeutic goals.

Important roles for platelets in regulating immunity have long been suspected. By adhering directly to immune and endothelial cells, platelets can coordinate leukocyte recruitment to sites of inflammation and tissue injury and therefore may play a role in vascular inflammation and sepsis (Rayes et al., 2020), as well as inflammatory events that promote tumor growth and metastasis (Stoiber and Assinger, 2020). These effects are largely confined to innate immune cells, but platelets also play a potentially important role in inhibiting Th17 cell differentiation through the release of either soluble factors such as platelet factor 4 or microparticles (Dinkla et al., 2016; Shi et al., 2014). Platelets are also especially strongly linked to asthma pathogenesis. Allergen challenge causes transient reductions in blood platelet counts, and platelet-leukocyte aggregates are readily found within the airways and lung tissue of asthma patients and in experimental systems (Pitchford et al., 2008; Shah et al., 2017; Sullivan et al., 2000). The distinct alpha and dense granules of platelets store either peptides or small molecules that powerfully influence eosinophil, neutrophil, dendritic cell (DC), T cell, and endothelial cell recruitment and activation. Platelet depletion or the pharmacologic disruption of platelet activation can inhibit asthmatic reactions experimentally (Pitchford et al., 2004; Suh et al., 2016). Nonetheless, prior to the present disclosure, little was known of how platelets are activated by allergens to influence Th2 and Th17 cell differentiation and how such differentiation occurs in the context of airway mycosis due to pathogenic fungi such as C. albicans.

The Wnt-beta catenin signaling pathway is activated in allergic airway disease and regulates this phenotype in complex ways (Kwak et al., 2015). A primary outcome of Wnt activation appears to be suppression of allergic airway disease (Beckert et al., 2018), although in the context of ultrafine particle challenge, Wnt activation may promote allergic disease (Harb et al., 2020). A suppressive role for Wnt was confirmed by the demonstration that Dkk-1 was required for allergic airway disease due to house dust mite (HDM) allergen (Chae et al., 2016b). However, whereas Dkk-1 influenced only Th2 cell responses in this context, it is demonstrated herein that Dkk-1 plays a much broader immune role, coordinately promoting both Th2 and Th17 cell responses, during airway mycosis due to C. albicans.

Thrombopoiesis occurs both in the bone marrow and the lung, with approximately 50% of megakaryocytes normally found in the lung (Lefrancais et al., 2017). Total lung megakaryocytes did not change after either C. albicans challenge or platelet depletion, but during fungal airway challenge, total megakaryocyte Dkk-1 decreased, while Dkk-1 in platelets, especially platelets isolated from the pulmonary circulation, increased. Megakaryocytes also express GP1bα, which is required for their normal development and function (Kanaji et al., 2004; Meinders et al., 2016). Without wishing to be bound by theory, these findings indicate that megakaryocytes respond to candidalysin through GP1bα by sequestering Dkk-1 into platelets, thus priming platelets to release enhanced quantities of Dkk-1 upon subsequent encounter with candidalysin. The specific ability of lung megakaryocytes as well as platelets to respond to C. albicans in a coordinated manner that critically protects the host suggests that, in some aspects, megakaryocytes evolved a partial lung residence to rapidly counter infections with the potential for invasion such as airway mycosis.

In summary, as disclosed herein, protective lung Th2 and Th17 cell responses against the common mucosa-associated fungus C. albicans can be coordinated through lung megakaryocytes and platelets. C. albicans can activate megakaryocytes and platelets through recognition of the virulence factor candidalysin by the VWF receptor GP1bα. Rather that eliciting thrombotic responses, candidalysin may instead promote secretion of Dkk-1, which drives the development of both Th2 and Th17 cells. In some aspects, C. albicans proteinases are less important for driving the asthma phenotype. Without wishing to be bound by theory, this may be because the proteinases are degrade fibrinogen completely and rapidly, such that fibrinogen cleavage products do not exist long enough to signal through TLR4/STAT6. Instead, in some aspects, the unique C. albicans product candidalysin may drive release of Dkk-1 from platelets by signaling through the receptor GP1bα.

The elaborate and highly specific defensive strategy that has evolved against C. albicans differs substantially from the mold proteinase-dependent pathway that also elicits Th2 and Th17 cell responses, confirming that C. albicans may be a major independent driver of human allergic diseases through adaptations that favor human infections (Kammer et al., 2020). For example, in the case of some molds, e.g., Aspergillus niger, the main virulence factor may be proteinases, which activate two pathways to promote expression of allergic airway disease: an innate immune pathway in which the proteinases may cleave fibrinogen into fibrinogen cleavage products that activate the transcription factor STAT6 via TLR4, which ultimately drives AHR and other aspects of the asthma phenotype; and an adaptive immune pathway in which the proteinases may activate platelets (e.g., by converting prothrombin to thrombin, which may then activate platelets via one or more platelet activated receptor (PAR)) to release Dkk-1 that drives the Th2 and Th17 responses. Thus, in some aspects, as for C. albicans, the Dkk-1 that is released drives the Th2 and Th17 responses as required to get a robust asthma phenotype.

### IV. Dkk-1 Compositions

Aspects of the disclosure concern compositions comprising an effective amount of one or more Dkk-1 inhibitors for treatment or prevention of an allergic airway disease. In particular aspects, a subject that has chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof is provided an effective amount of a composition comprising one or more Dkk-1 inhibitors or one or more functional derivatives thereof.

In some aspects, there are compositions comprising one or more Dkk-1 inhibitors and one or more allergic disease therapies. In such cases, the one or more Dkk-1 inhibitors and one or more allergic disease therapies may or may not be in the same formulation and may or may not be configured to be delivered by the same route of administration.

In some aspects, the Dkk-1 inhibitors inhibit Dkk-1 activity. In other aspects, the Dkk-1 inhibitors inhibit release of Dkk-1 from platelets. In specific aspects, the Dkk-1 inhibitors inhibit release of Dkk-1 from platelets without compromising the essential clotting/thrombosis activity of platelets.

The one or more Dkk-1 inhibitors may comprise a small molecule, an antibody, or a nucleic acid. Small molecule inhibitors of Dkk-1 include but are not limited to WAY 262611, gallocyanine, NCI8642, or functional derivatives thereof. Antibody inhibitors of Dkk-1 include but are not limited to DKN-01 or a functional derivative thereof.

The compositions of one or more Dkk-1 inhibitors may or may not be tailored to address any symptom of an allergic airway disease or to enhance a subject's response to an allergic airway disease. The compositions may be given to a subject without having prior analysis of their immune system. The compositions of one or more Dkk-1 inhibitors may comprise any one or more Dkk-1 inhibitors associated with efficacious therapy to treat or prevent an allergic airway disease.

The subject may be given one or more compositions of one or more Dkk-1 inhibitors, including compositions that comprise one or more Dkk-1 inhibitors that overcome or address any symptom of an allergic airway disease. Symptoms can include but are not limited inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof. The Dkk-1 inhibitors and/or derivatives thereof may be given to treat or prevent an allergic airway disease and/or enhance therapy to treat or prevent an allergic airway disease.

The compositions of one or more Dkk-1 inhibitors can be administered alone or in combination with one or more additional allergic airway disease therapies comprising but not limited to corticosteroids, leukotriene modifiers, bronchodilators, antifungals and/or antibiotics, biologics, allergy shots, antihistamines, decongestants, cromolyn, or combinations thereof. In specific aspects, the corticosteroids comprise fluticasone, dexamethasone, budesonide, mometasone, beclomethasone, ciclesonide, or combinations or pharmaceutically acceptable derivatives thereof. In specific aspects, the leukotriene modifiers comprise montelukast, zafirlukast, zileuton, or combinations or pharmaceutically acceptable derivatives thereof. In specific aspects, the bronchodilators comprise theophylline, albuterol, levalbuterol, ipratropium, aclidinium, arformoterol, formoterol, indacaterol, tiotropium, salmeterol, glycopyrrolate, olodaterol, vilanterol, umeclidinium, or combinations or pharmaceutically acceptable derivatives thereof. In specific aspects, the antifungals and/or antibiotics comprise amphotericin B, azithromycin, terbinafine, voriconazole, itraconazole, fluconazole, isavuconazole, posaconazole, ketoconazole, micafungin, ibrexafungerp, caspofungin, or combinations or pharmaceutically acceptable derivatives thereof. In specific aspects, the biologics comprise omalizumab, mepolizumab, benralizumab, dupilumab, reslizumab, or combinations or pharmaceutically acceptable derivatives there. In specific aspects, the antihistamines comprise azelastine, brompheniramine, cetirizine, chlorpheniramine, desloratadine, diphenhydramine, doxylamine, fexofenadine, hydroxyzine, ketotifen, loratadine, levocetirizine, olaptadine, or combinations or pharmaceutically acceptable derivatives there. In specific aspects, the decongestants comprise levmetamfetamine, naphazoline, pseudoephedrine, phenylephrine, propylhexedrine, oxymetazoline, xylometazoline, or combinations or pharmaceutically acceptable derivatives there.

Administration “in combination with” one or more additional therapeutic agents includes both simultaneous (at the same time) and consecutive administration in any order. The compositions of Dkk-1 inhibitors and/or derivatives thereof and one or more additional therapeutic agents can be administered in one composition, or simultaneously as two separate compositions, or sequentially. Administration can be chronic or intermittent, as deemed appropriate by the supervising practitioner, including in view of any change in any undesirable side effects.

An effective amount of one or more Dkk-1 inhibitors may be provided to a subject in need thereof, such as a subject that has an allergic airway disease, has developed or acquired risk factors for developing allergic airway disease, or is suspected of having an allergic airway disease, and may or may not be provided with one or more additional allergic airway disease therapies. The additional allergic airway disease therapies may be in the same composition as the one or more Dkk-1 inhibitors, or the additional allergic airway disease therapies may be in a different composition as the one or more Dkk-1 inhibitors. The additional allergic airway disease therapies may or may not be given to the subject at the same time as the one or more Dkk-1 inhibitors and/or derivatives thereof. The additional allergic airway disease therapies may assist in treating and/or preventing at least one allergic airway disease or symptom thereof and/or the additional allergic airway disease therapies may be useful for treating and/or preventing at least one symptom of another medical condition.

In cases where the subject is provided with two or more doses of the composition, the duration between the administrations should be sufficient to allow time for distribution and effect in the individual, and in specific aspects the duration between doses is at most, at least, equal to, or between any two of 1, 2, 3, 4, 5, 6, 7, or more days. In some cases, the duration between administrations is 1-24 hours, 1-7 days, 1-4 weeks, 1-12 months, or more, or any range derivable there between. In some aspects, the one or more Dkk-1 inhibitors are administered prior to the one or more additional allergic airway disease therapies. In some aspects, the one or more Dkk-1 inhibitors are administered at least, at most, equal two, or between any two of 1, 2, 3, 5, 6, 12, 24 hours or 1, 2, 3, 4, 6, 7, 8, 9, 10, 11, 12, 13, 14 days or 1, 2, 3, 4, 5, 6, 7, or 8, 9, 10, 11, 12 weeks or 1, 2, 3, 4, 5, 6, 7, or 8, 9, 10, 11, 12 months (or any derivable range therein) prior to the one or more additional allergic airway disease therapies. In some aspects, the one or more Dkk-1 inhibitors are administered after one or more additional allergic airway disease therapies. In some aspects, the one or more Dkk-1 inhibitors are administered at least, at most, equal two, or between any two of 1, 2, 3, 5, 6, 12, 24 hours or 1, 2, 3, 4, 6, 7, 8, 9, 10, 11, 12, 13, 14 days or 1, 2, 3, 4, 5, 6, 7, or 8, 9, 10, 11, 12 weeks or 1, 2, 3, 4, 5, 6, 7, or 8, 9, 10, 11, 12 months (or any derivable range therein) after the one or more additional allergic airway disease therapies.

Compositions comprising the one or more Dkk-1 inhibitors and/or one or more allergic airway disease therapies are provided. The compositions may include a pharmaceutically acceptable carrier, diluent, and/or excipient, in some cases. In particular cases, the compositions comprising one or more Dkk-1 inhibitors and/or one or more allergic airway disease therapies are formulated to target a region in the respiratory tract, including any portion of the respiratory tract. The compositions comprising one or more Dkk-1 inhibitors and/or one or more allergic airway disease therapies are formulated to be delivered to any portion of the respiratory tract.

The compositions comprising one or more Dkk-1 inhibitors and/or one or more allergic airway disease therapies encompassed herein may be administered in a variety of ways known or available to those skilled in the art. In one aspect of any of the above methods involving administration of compositions comprising one or more Dkk-1 inhibitors and/or one or more allergic airway disease therapies, the composition is directly or indirectly delivered to the respiratory tract of the subject. In one aspect, the compositions comprising one or more Dkk-1 inhibitors and/or one or more allergic airway disease therapies are administered to the subject by an oral or intranasal route (e.g., inhaled). In one aspect, the compositions comprising one or more Dkk-1 inhibitors and/or one or more allergic airway disease therapies are delivered to the subject in a form of a liquid, foam, cream, spray, powder, or gel. In one aspect, the compositions comprising one or more Dkk-1 inhibitors and/or one or more allergic airway disease therapies comprise a buffering agent, along with preservatives, stabilizers, binders, compaction agents, lubricants, dispersion enhancers, disintegration agents, antioxidants, flavoring agents, sweeteners, and coloring agents.

In addition, the composition can administered alone or in combination with a carrier, such as a pharmaceutically acceptable carrier or a biocompatible scaffold. In some cases, formulations of the composition comprise an ingestible carrier, which may be a pharmaceutically acceptable carrier such as a capsule, tablet or powder. The formulation of the composition may further comprise an adjuvant, a drug, a biological compound, or a mixture thereof.

Oral formulations include such normally employed excipients such as, for example, pharmaceutical grades of mannitol, lactose, starch, magnesium stearate, sodium saccharine, cellulose, magnesium carbonate and the like. These formulations take the form of solutions, suspensions, tablets, pills, capsules, sustained release formulations or powders and contain about 10% to about 95% of active ingredient, for example, about 25% to about 70%.

In some aspects, the compositions are formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

In some aspects, the disclosed compositions are prepared as a tablet. The tablet may include the one or more Dkk-1 inhibitors or other actives and one or more tableting agents, such as dibasic calcium phosphate, stearic acid, croscarmellose, silica, cellulose and cellulose coating. The tablets may be formed using a direct compression process, though those skilled in the art will appreciate that various techniques may be used to form the tablets.

In one aspect, the disclosed compositions are formulated as a capsule. The capsule may be a hollow, generally cylindrical capsule formed from various substances, such as gelatin, cellulose, carbohydrate or the like.

While the compositions of the present disclosure may be formulated for oral administration, other routes of administration can be employed, however, including, but not limited to, intranasal, subcutaneous, intramuscular, intradermal, transdermal, intraocular, intraperitoneal, mucosal, vaginal, rectal, and intravenous.

In specific aspects, the compositions comprising one or more Dkk-1 inhibitors and/or one or more allergic airway disease therapies and related methods of the present disclosure utilize particular formulation methods. In some examples, the carrier may further comprise a disintegrant, a glidant, and/or a lubricant, such as is described in U.S. Pat. No. 9,084,434, for example, to facilitate having a greater shelf life and/or half-life of the formulation. The disintegrant may be any suitable disintegrant such as, for example, a disintegrant selected from the group consisting of sodium croscarmellose, crospovidone, gellan gum, hydroxypropyl cellulose, starch, and sodium starch glycolate. The glidant may be any suitable glidant such as for example, a glidant selected from the group consisting of silicon dioxide, colloidal silicon dioxide, and talc. The lubricant may be any suitable lubricant such as for example, a lubricant selected from the group consisting of calcium stearate, magnesium stearate, stearic acid, sodium stearyl fumerate, and vegetable based fatty acids. In the composition and method of the present disclosure, the carrier, is present in the composition in a range of approximately 30% w/w to approximately 98% w/w; this weight percentage is a cumulative weight percentage taking into consideration all ingredients present in the carrier. The composition of the present disclosure may be an oral dosage form, a powder that is mixed into a liquid, or a chewing gum. Where the composition is an oral dosage form, the oral dosage form may be selected from the group consisting of tablets, caplets, and capsules, wherein the tablets and caplets may be solid or chewable. Where the composition is a powder, it may be mixed into a liquid that is selected from the group consisting of water, milk, juice, and yogurt. Where the composition is a chewing gum, the gum may be soft gum or hard chewing gum tablets.

In particular cases the formulation of the compositions comprising one or more Dkk-1 inhibitors and/or one or more allergic airway disease therapies comprises one or more excipients. Examples of excipients that may be used to formulate appropriate dosage forms include binders, disintegrants, lubricants, coatings, plasticizers, compression agents, wet granulation agents, and sweeteners, all of which are known to those of ordinary skill in the art to which the disclosure pertains. All of the following examples are provided by way of illustration and not limitation. Binders are used where appropriate to help the dosage form ingredients still together. Examples of binders include carbopol, povidone, and xanthan gum. Lubricants are generally always used in the manufacture of dosage forms by direct compression in order to prevent the compacted powder mass from sticking to the equipment during the tabletting or encapsulation process. Examples of lubricants include calcium stearate, magnesium stearate, stearic acid, sodium stearyl fumerate, and vegetable based fatty acids. Disintegrants aid in the break-up of the compacted mass when placed in a fluid environment. Examples of disintegrants include sodium croscarmellose, crospovidone, gellan gum, hydroxypropyl cellulose, starch, and sodium starch glycolate. Coatings are used to control the solubility of the drug. Examples of coatings include carrageenan, cellulose acetate phthalate, ethylcelulose, gellan gum, matodextrin, methacrylates, methylcellulose, microcrystalline cellulose, and shellac. Plasticizers are used to control the release rate of the drug from the dosage form. Examples of plasticizers include citrate esters, dibutyl sebacate, diethyl phthalate, polyvinylacetate phthalate, and triacetin. Compression agents include calcium carbonate, dextrose, fructose, guar gum, honey, lactose, maltodextrin, maltose, mannitol, microcrystalline cellulose, molasses, sorbitol, starch, and sucrose. Wet granulation agents include calcium carbonate, lactose, maltodextrin, mannitol, microcrystalline cellulose, povidone, and starch. Sweeteners include aspartame, dextrose, fructose, honey, lactose, maltodextrin, maltose, mannitol, molasses, monoammonium glycyrrhizinate, sorbitol, sucralose, and sucrose. Excipients that are generally used in the manufacture of chewable tablets include by way of illustration and not limitation, dextrose, fructose, guar gum, lactose, maltodextrin, maltose, mannitol, microcrystalline cellulose, and sorbitol. As is evident from the foregoing list, many of the same ingredients may be used for various different purposes in various different dosage forms.

The agents of the disclosure may be administered by the same route of administration or by different routes of administration. In some aspects, the one or more Dkk-1 inhibitors are administered intravenously, intramuscularly, subcutaneously, topically, orally, transdermally, intraperitoneally, intraorbitally, by implantation, by inhalation, intrathecally, intraventricularly, by aerosol, or intranasally. In some aspects, the one or more additional allergic airway disease therapies are administered intravenously, intramuscularly, subcutaneously, topically, orally, transdermally, intraperitoneally, intraorbitally, by implantation, by inhalation, intrathecally, intraventricularly, by aerosol, or intranasally. The appropriate dosage may be determined based on the type of disease to be treated, severity and course of the disease, the clinical condition of the subject, the subject's clinical history and response to the treatment, and the discretion of the attending physician.

The treatments may include various “unit doses.” Unit dose is defined as containing a predetermined-quantity of the therapeutic composition calculated to produce the desired responses discussed above in association with its administration, i.e., the appropriate route and regimen. The quantity to be administered, and the particular route and formulation, is within the skill of determination of those in the clinical arts and depends on the result and/or protection desired. A unit dose need not be administered as a single injection but may comprise continuous infusion over a set period of time. In some aspects, a unit dose comprises a single administrable dose.

The quantity of one or more Dkk-1 inhibitors and/or one or more allergic airway disease therapies to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about at most, at least, equal to, or between any two of 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 PM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at most, at least, equal to, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Typically, compositions are administered in a manner compatible with the dosage formulation, and in such amount as will be therapeutically or prophylactically effective for the subject being treated. Precise amounts of the therapeutic composition also depend on the judgment of the practitioner and are peculiar to each individual. Suitable regimes for initial administration and boosters are also variable, but are typified by an initial administration followed by subsequent administrations. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

The compositions will be pharmaceutically acceptable or pharmacologically acceptable. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters. The use of such media and agents for pharmaceutical active substances is well known in the art. Except insofar as any conventional media or agent is incompatible with the active ingredients, its use in immunogenic and therapeutic compositions is contemplated.

### V. Methods of Treatment and Use of the Disclosure

In some aspects, the present disclosure provides methods for allergic airway disease treatment that employs one or more inhibitors of Dkk-1, comprising administering an effective amount of one or more Dkk-1 inhibitors of the present disclosure. The allergic airway disease may be resistant to treatment with corticosteroids. In one aspect, methods are encompassed herein for treating, delaying progression of, delaying onset of, or reducing the risk of getting an allergic airway disease in an individual by administering to the individual an effective amount the one or more inhibitors of Dkk-1.

The present methods may be applied for the treatment of allergic airway diseases of any cause. For example, the allergic airway disease may be caused at least in part by one or more irritants and/or activators of an immune response comprising fungal infection, mold or mildew, hair or dander, dust, pollen, smoke, exercise, stress, perfume or other strong odors, air pollution comprising ozone, particulates, nitrogen dioxides, or sulfate aerosols, and/or changes in the weather. In specific cases, the allergic airway disease is caused at least in part by fungal infection. The fungal infection may be caused by any pathogenic fungal species, any or all of which could cause allergic airway disease. In some aspects, the fungal infection comprises Aspergillus spp., Penicillium spp., Alternaria spp., Penicillium spp., Curvularia spp., Bipolaris, Mucor spp., Rhizopus spp., Pneumocystis spp., Aureobasidia spp., Cladosporium spp., Cochliobus spp., Paecilomyces spp., Trichoderma spp., Trichosporon spp., Malassezia spp., and/or Candida spp. fungi. In certain aspects, the fungal infection comprises Aspergillus spp. In specific aspects, the Aspergillus fungi comprise Aspergillus flavus, Aspergillus fumigatus, Aspergillus nidulans, Aspergillus oryzae, Aspergillus sydowii, Aspergillus versicolor, Aspergillus wentii, and/or Aspergillus niger. In certain aspects, the fungal infection comprises Candida spp. In specific aspects, the Candida fungi comprise Candida albicans, Candida tropicalis, Candida glabrata, Candida auris, Candida lusitaniae, Candida parapsilosis, Candida krusei, Candida dubliniensis, and/or Candida guilliermondii.

The subject may be undergoing treatment for an allergic airway disease. The treatment may comprise a corticosteroid, and in some aspects, the allergic airway disease may be found to be resistant to treatment with the corticosteroid. The subject may have been previously treated for an allergic airway disease. The previous treatment may have been a corticosteroid, and in some aspects, the allergic airway disease was found to be resistant to treatment with the corticosteroid. The present methods may be applied for the treatment of allergic airway diseases that are resistant to treatment with corticosteroids.

The allergic airway disease may be chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof. In specific cases, the allergic airway disease is asthma. The individual may be at risk for allergic airway disease, including over the general population, and the individual at risk for allergic airway disease may be so because of a personal or family history or because the individual has exposure to one or more irritants and/or immune activators, including fungal infection, mold or mildew, hair or dander, dust, pollen, smoke, exercise, stress, perfume or other strong odors, air pollution comprising ozone, particulates, nitrogen dioxides, or sulfate aerosols, and/or changes in the weather.

In particular aspects, a subject that is the subject for methods and compositions of the disclosure has a medical condition in which at least one symptom is inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof. In specific aspects, the subject has inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof. In specific aspects, a treatment regimen is for a subject with an allergic airway disease having inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof. In specific aspects, a treatment regimen is for a subject with chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof.

A subject having an allergic airway disease is provided an effective amount of a composition comprising at least an effective amount of one or more Dkk-1 inhibitors to treat or prevent the allergic airway disease, which may have as a symptom inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof. As used herein, the term “therapeutically effective amount” is synonymous with “effective amount,” “therapeutically effective dose,” and/or “effective dose,” and refers to an amount of an agent sufficient to produce a desired result or exert a desired influence on the particular condition being treated. In some aspects, a therapeutically effective amount is an amount sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. For example, treatment of an allergic airway disease may involve a reduction in inflammation, a reduction in airway hyper-responsiveness, inhibition of an aberrant adaptive or innate immune response, prevention of inflammation, airway hyper-responsiveness, and/or an aberrant or innate adaptive immune response, or delay in onset of inflammation, airway hyper-responsiveness, and/or an aberrant adaptive or innate immune response. The effective amount may vary depending on the organism or individual treated. The appropriate effective amount to be administered for a particular application of the disclosed methods can be determined by those skilled in the art, using the guidance provided herein. As used herein, the terms “treatment,” “treat,” or “treating” refers to intervention in an attempt to alter the natural course of the subject being treated, and may be performed either for prophylaxis or during the course of pathology of a disease or condition. Treatment may serve to accomplish one or more of various desired outcomes, including, for example, preventing occurrence or recurrence of disease, alleviation or reduction in severity of symptoms, and diminishment of any direct or indirect pathological consequences of the disease, preventing disease spread, lowering the rate of disease progression, amelioration or palliation of the disease state, and remission or improved prognosis. In addition, “treating” or “treatment” does not require complete alleviation of signs or symptoms, does not require a cure, and specifically includes protocols that have only a marginal effect on the patient.

In some cases, there may be a regimen having a first phase for an initial treatment, which may last for days, weeks, months, or years, and then another phase for maintenance, which may last for weeks, months, or years. Such a treatment may be administered one or more times a day, including about 1, 2, 3, or more times per day, for a period sufficient to stabilize effects of the initial treatment, such as reduced inflammation, reduced airway hyper-responsiveness, and/or inhibited adaptive or innate immune response in the airway of a subject. In other cases, in a maintenance phase, a subject is provided a lesser amount of the Dkk-1 inhibitor and/or fewer administrations than the initial treatment phase. The duration of a treatment regimen may be dependent on each individual patient and the stage of the medical condition. In some cases, a continued treatment for a certain period of time occurs until a detectable improvement in an allergic airway disease.

In specific aspects, a subject is provided one or more additional allergic airway therapies in addition to the Dkk-1 inhibitors encompassed herein. In some cases, the improved allergic airway disease is maintained by additional treatment, such as one or more additional allergic airway disease therapies, although the additional treatment may be reduced in frequency and/or volume. For example, they may be provided the following in addition to and/or in combination with compositions comprising one or more Dkk-1 inhibitors: Corticosteroids, leukotriene modifiers, bronchodilators, antifungals, biologics, allergy shots, antihistamines, decongestants, cromolyn, or combinations thereof. The one or more additional allergic airway therapies may improve or enhance treatment of the allergic away disease by the one or more Dkk-1 inhibitors as compared to treatment of the allergic airway disease with only one or more Dkk-1 inhibitors.

In a particular form of the disclosure, an initial treatment regimen comprising an effective amount of a composition comprising at least an effective amount of one or more Dkk-1 inhibitors may comprise at least about, at least, at most, exactly, or between any two of 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300,400, 500, 1000 μg/kg, mg/kg, μg/day, or mg/day or any range or value derivable therein.

In a particular form of the disclosure, an initial treatment regimen comprising an effective amount of a composition comprising at least an effective amount of one or more additional allergic airway disease therapies may comprise at least about, at least, at most, exactly, or between any two of 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein.

In a particular form of the disclosure, an initial treatment regimen comprises an effective amount of a formulation comprising at least an effective amount of one or more Dkk-1 inhibitors, which may comprise of at least about, at least, at most, exactly, or between any two of 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein, and at least an effective amount of one or more additional allergic airway disease therapies, which may comprise at least about, at least, at most, exactly, or between any two of 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein.

In some aspects, disclosed herein are methods of treating or preventing an allergic airway disease in a subject by administering an effective amount of a composition comprising one or more Dkk-1 inhibitors. The subject may have or may be at risk of having chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof. The subject may have or be at risk of having inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof. The subject may be at risk because of a personal or family history or because the individual has exposure to one or more irritants and/or immune activators, including fungal infection, mold or mildew, hair or dander, dust, pollen, smoke, exercise, stress, perfume or other strong odors, air pollution comprising ozone, particulates, nitrogen dioxides, or sulfate aerosols, and/or changes in the weather.

In certain aspects, at least one symptom of at least one allergic airway disease is treated with an effective amount one or more Dkk-1 inhibitors. A subject may be treated for inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof, by providing an effective amount of a composition comprising one or more Dkk-1 inhibitors.

In some aspects, the subject is at a higher risk than an average person in the general population. In some aspects, the medical condition in which at least one symptom is inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof poses a greater risk to the health or life of the subject than such a condition would pose to an average person in the general population. In some cases, the method is employed for a subject where it is uncertain whether or not risk of developing a medical condition in which at least one symptom is inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof, is increased, whereas in other cases the method is employed for a subject where it is known that the risk of developing a medical condition in which at least one symptom is inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof, is increased. In other cases, it has been determined that the risk of developing a medical condition in which at least one symptom is inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof, is increased for the subject, but the methods of the disclosure are still employed as a routine matter or in the general therapeutic interest of the subject.

In some cases, the effective amount of the composition comprising one or more Dkk-1 inhibitors indirectly or directly improves inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof because it indirectly or directly reduces airway inflammation, reduces airway hyper-responsiveness, and/or inhibits an adaptive or innate immune response in the airway of the subject. In some aspects, inhibiting the adaptive or innate immune response by the subject comprises inhibiting cytokine secretion and/or inhibiting recruitment or activity of inflammatory cells and/or T helper effector cells. In specific aspects, the cytokines comprise interleukin-2, interleukin-4, interleukin-5, interleukin-6, interleukin-9, interleukin-10, interleukin-12, interleukin-13, interleukin-17A, interleukin-17B, interleukin-17C, interleukin-17D, interleukin-17E, interleukin-17F, interleukin-22, interleukin-33, tumor necrosis factor, thymic stromal lymphopoietin, ciliary neurotrophic factor, or interleukin-1l. In specific aspects, the inflammatory cells comprise granulocytes and/or macrophages. In specific aspects, the T helper effector cells comprise T helper type 2 (Th2) cells and/or T helper type 17 (Th17) cells.

### VI. Sample Collection and Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy, or skin biopsy. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, sputum, nasal lavage, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: aspirating, scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple lung samples may be obtained for diagnosis by the methods described herein. In other cases, multiple samples, such as one or more samples from one tissue type (for example lung) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, a sputum sample, a nasal lavage sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of an esophageal or a suspected esophageal tumor or neoplasm. In some cases, the fine needle aspirate sampling procedure may be guided by the use of an ultrasound, X-ray, or other imaging device.

In some aspects of the present methods, the biological sample may be obtained from a subject directly, from a medical professional, from a third party, or from a kit provided by a third party. In some cases, the subject, a medical professional, or a third party may be provided with suitable containers and excipients for storage and transport of the biological sample.

In some aspects of the methods described herein, a medical professional need not be involved in the initial sample acquisition. A subject may alternatively obtain a sample through the use of an over the counter (OTC) kit. An OTC kit may contain a means for obtaining said sample as described herein, a means for storing said sample for inspection, and instructions for proper use of the kit. A sample suitable for use may be any material containing tissues, cells, nucleic acids, genes, gene fragments, expression products, gene expression products, or gene expression product fragments of a subject to be tested. Methods for determining sample suitability and/or adequacy are known in the art.

In some aspects, the subject may be referred to a specialist such as an oncologist, surgeon, or endocrinologist. The specialist may likewise obtain a biological sample for testing or refer the subject to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### VII. Articles of Manufacture or Kits

An article of manufacture or a kit comprising compositions of one or more Dkk-1 inhibitors and/or one or more additional allergic airway disease therapies is also provided herein. The Dkk-1 inhibitors may be a small molecule, an antibody, or a nucleic acid. The one or more additional therapies may be corticosteroids, leukotriene modifiers, bronchodilators, antifungals, biologics, allergy shots, antihistamines, decongestants, cromolyn, or combinations thereof. The kit may comprise the Dkk-1 inhibitors and/or additional allergic airway disease therapies together or separate, buffers, salts, directions for use, or a combination thereof.

The article of manufacture or kit can further comprise a package insert comprising instructions for using the compositions to treat or delay progression of allergic airway disease in an individual. Any of the Dkk-1 inhibitors and/or additional allergic airway disease therapies described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or a nickel-molybdenum alloy). In some aspects, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some aspects, the article of manufacture further includes one or more of another agent (e.g., a chemotherapeutic agent, and anti-neoplastic agent). Suitable containers for the one or more agent include, for example, bottles, vials, bags and syringes.

### EXAMPLES

The following examples are included to demonstrate preferred aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1

C. albicans Elicits Allergic Disease Through a Distinct Immunological Mechanism

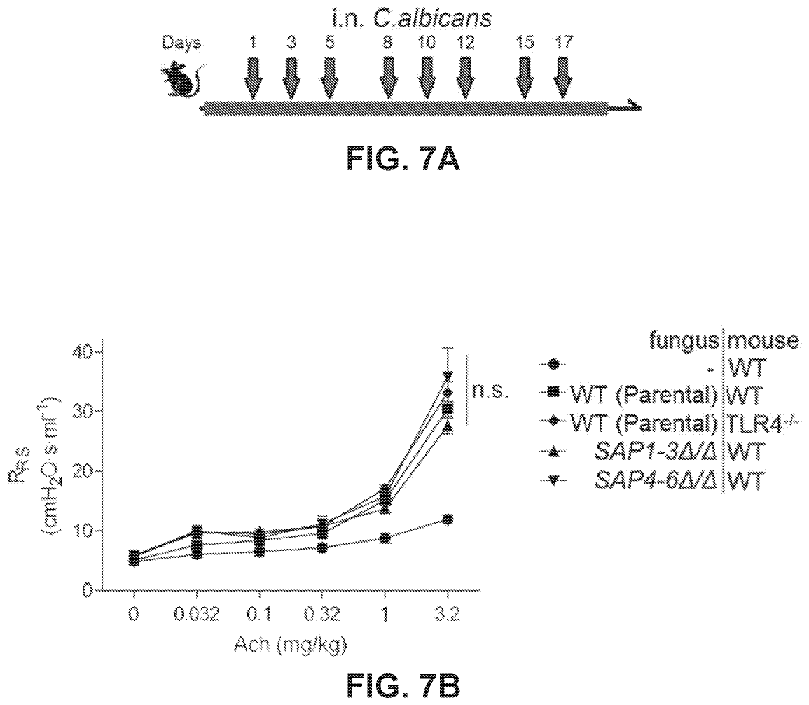

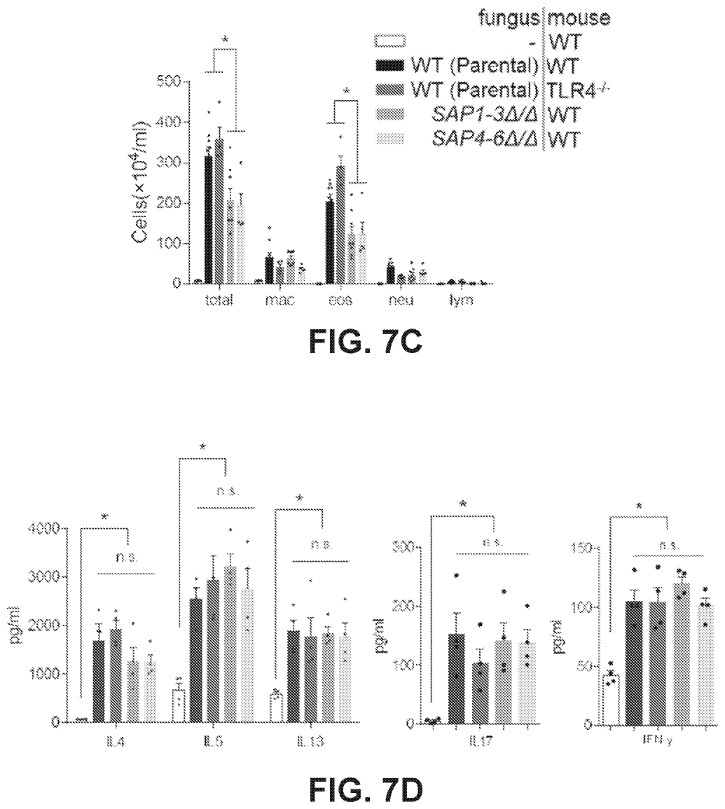

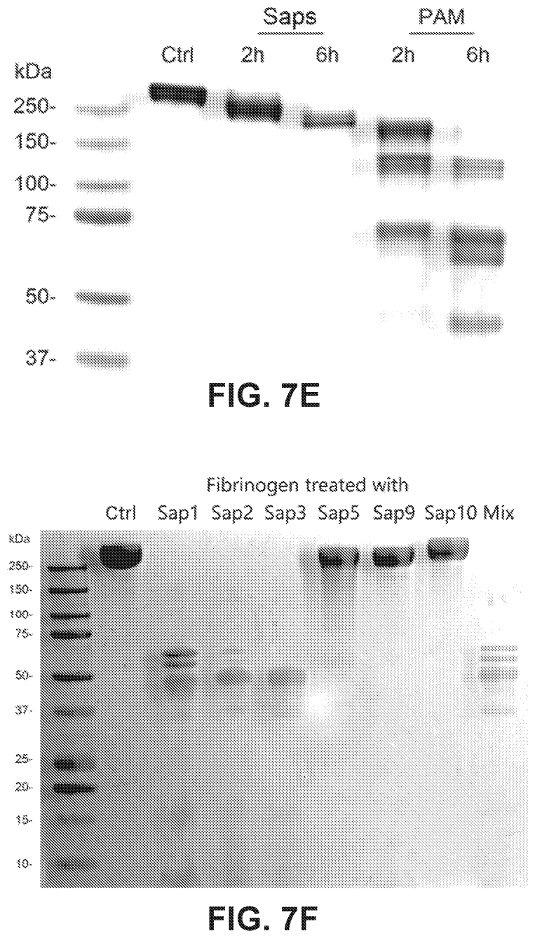

C. albicans may induce allergic airway disease through the proteinase-FCP-TLR4 signaling pathway that is required for allergic disease induced by molds such as A. niger (Millien et al., 2013). Wildtype mice challenged intranasally with viable wildtype (parental strain) and secreted aspartic proteinase (Sap)-deficient C. albicans cells were assessed for key allergic airway disease features 24 h following the final challenge (FIG. 7A). In contrast to proteinase-deficient A. niger (Porter et al., 2009), C. albicans cells lacking broad subsets of SAP genes (SAP1-3 or SAP4-6) induced equivalent airway hyper-responsiveness as assessed by increases in respiratory system resistance (R_{R}S) following provocative challenge with escalating doses of acetylcholine comparable to that of isogenic wildtype cells (FIGS. 7A-7B). Although significant decreases in total inflammatory cells and eosinophils in bronchoalveolar lavage fluid (BALF; FIG. 7C) were observed, no significant differences were observed regarding Th cell-related cytokines secreted from deaggregated lung (FIG. 7D).

In contrast to studies of A. niger-exposed mice (Millien et al., 2013), no reduction in airway hyper-responsiveness, BALF cell counts, or secreted cytokines was observed for lung homogenates of Tlr4^{-/-} mice challenged with wildtype C. albicans as compared to syngeneic wildtype mice as compared to isogenic wildtype fungi (FIGS. 7A-7D). These observations suggest that, in some aspects, the major Saps from C. albicans do not generate FCPs that can signal through TLR4 to drive the allergic airway disease phenotype. To confirm this, proteolytic activities of the highly allergenic proteinases from A. melleus (PAM (Landers et al., 2019b)) were compared with Saps with respect to fibrinogen hydrolysis. Whereas PAM yielded fibrinogen cleavage products of the expected size (100-150 kDa) (Landers et al., 2019b), purified Saps hydrolyzed fibrinogen without generating detectable FCPs after 2 and 6 h of incubation (FIG. 7E). Recombinant Saps, especially Saps 1, 2 and 3, did generate FCPs, but of smaller molecular size (˜37-65 kDa) as compared to PAM (˜70-150 kDa; FIG. 7F). Thus, unlike molds such as A. niger, C. albicans induces allergic airway disease through a mechanism operating substantially independently of secreted fungal proteinases and TLR4.

### Example 2

### Candidalysin Drives Allergic Airway Disease

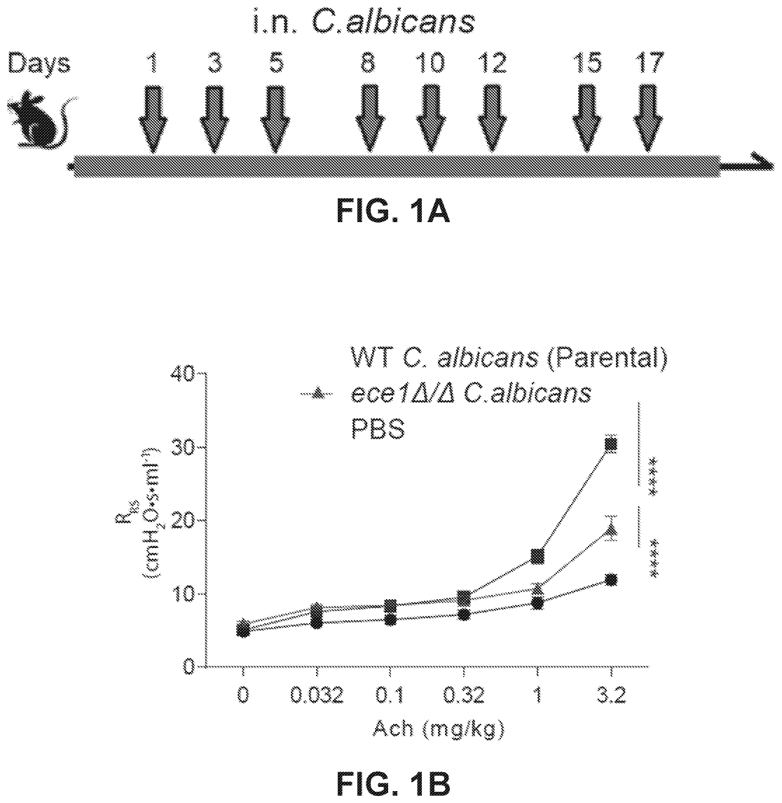

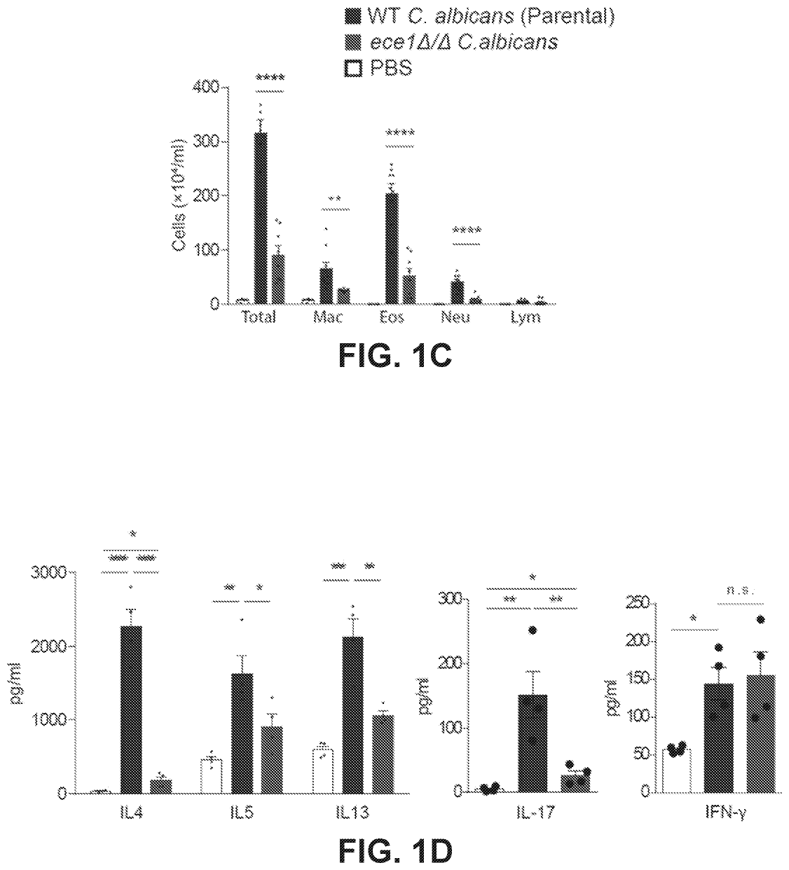

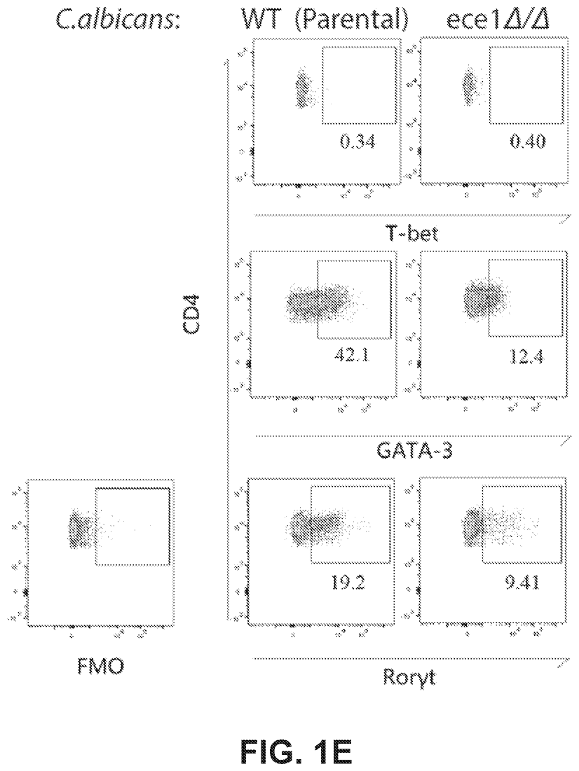

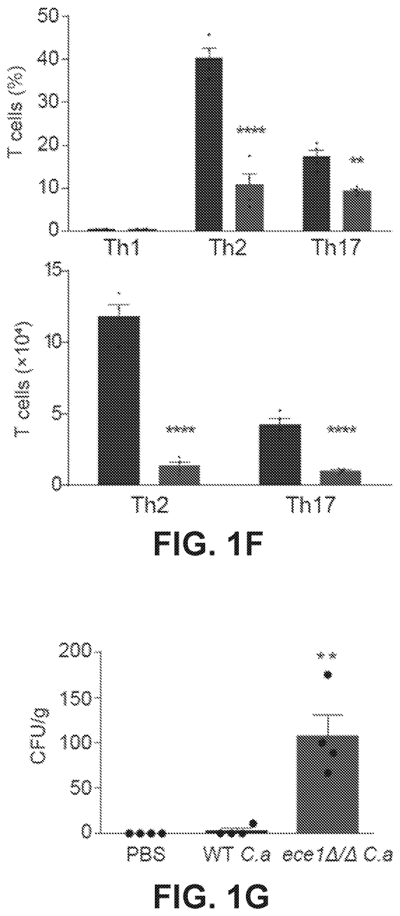

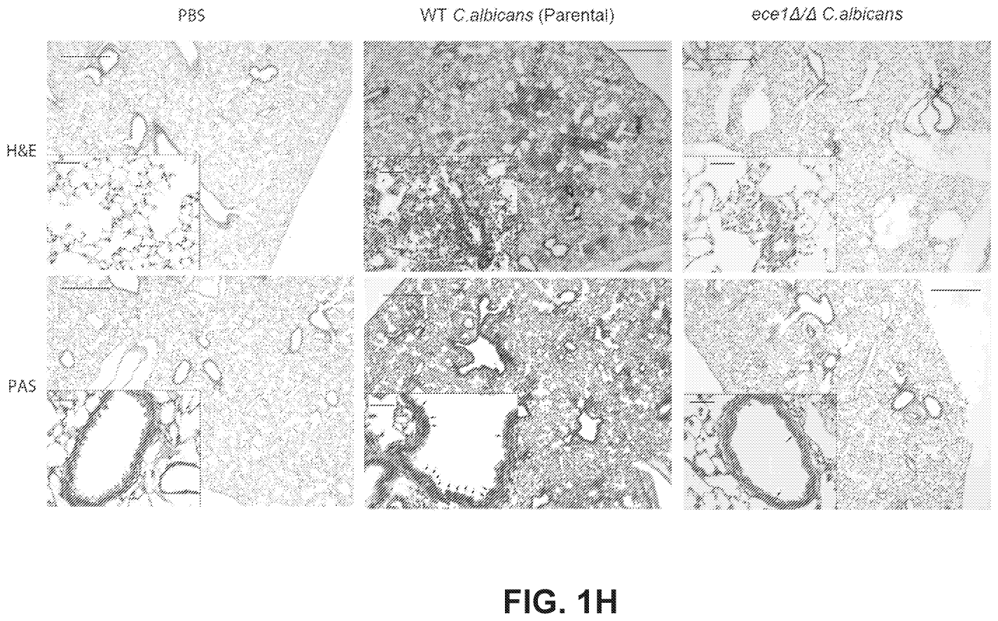

The importance of other virulence factors were also considered for this phenotype. C. albicans expresses several virulence factors in addition to proteinases, including candidalysin, a non-proteinase peptide toxin secreted by hyphal cells that is a potent innate immune activator and mediator of Th17 cell responses (Ho et al., 2020; Kasper et al., 2018; Moyes et al., 2016; Verma et al., 2017; Verma et al., 2018). Therefore, the importance of candidalysin for C. albicans-mediated allergic airway disease was investigated by comparing wildtype mice challenged with isogenic wildtype yeast cells (parental strain) or with candidalysin-deficient ece1Δ/Δ yeasts. In contrast to proteinase-deficient cells, ece1Δ/Δ C. albicans cells induced significantly less airway hyper-responsiveness (FIGS. 1A-1B) and substantially reduced BALF cellularity marked especially by fewer macrophages, eosinophils, and neutrophils (FIG. 1C).

Similarly, type 2 cytokine (interleukin (IL)-4, IL-5 and IL-13) and IL-17 secretion from deaggregated lung was significantly reduced in mice receiving ece1Δ/Δ C. albicans, but secretion of gamma interferon (IFN-γ) was not affected (FIG. 1D). ece1Δ/Δ C. albicans also failed to induce robust secretion of the innate proinflammatory cytokines IL-1β and IL-6, although TNF secretion was unaffected by the lack of candidalysin (data not shown). These findings were paralleled by reductions in whole lung of major T helper effector subsets that have been linked to human and experimental asthma, especially T helper type 2 cell (Th2; GATA3⁺ (Zheng and Flavell, 1997)) and Th17 cell (RORγt+(Ivanov et al., 2006)) cells as assessed by flow cytometry (FIGS. 1E-1F). T-bet+(Szabo et al., 2000) T_{H}1 cells were not detected, suggesting that, in some aspects, the secreted IFN-γ (FIG. 1D) derived from non-Th cell sources. These cytokine profiles suggested that, in some aspects, in contrast to C. albicans Saps, candidalysin can promote innate immune responses (Moyes et al., 2016; Verma et al., 2017) _ENR_6, but can also induce type 2 and type 17-biased inflammation in the context of C. albicans-induced allergic airway disease.

T helper effector cells and their cytokines have all previously been shown to mediate protection against fungal infections, including airway mycosis that is often the underlying cause of asthma and other allergic airway diseases (Ma et al., 2008; Porter et al., 2011a; Porter et al., 2011b; Porter et al., 2009; Porter et al., 2014). The diminished induction of these protective responses by ece1Δ/Δ C. albicans suggested that this mutant might be poorly cleared from the lung. Indeed, whereas wildtype C. albicans was almost completely cleared from lungs 24 h after the final intranasal challenge, approximately 100 CFU/g were recovered from the lungs of each mouse challenged with ece1Δ/Δ C. albicans cells (FIG. 1G).

Microscopically, wildtype C. albicans-challenged lungs were marked by the accumulation of inflammatory cells, including neutrophils and eosinophils, in the alveoli and especially in a peri-bronchovascular distribution. In comparison, ece1Δ/Δ C. albicans-challenged lungs showed minimal peri-bronchovascular inflammation (FIG. 1H). As revealed by periodic acid-Schiff staining, goblet cells, a prominent example of airway remodeling that is typical of asthma (Fahy, 2001), were abundant in the airway epithelium of mice challenged with WT C. albicans, but scant in the lung epithelium after ece1Δ/Δ C. albicans challenge (FIG. 1H). Thus, candidalysin can drive both robust allergic airway disease and efficient clearance of C. albicans from mouse lung.

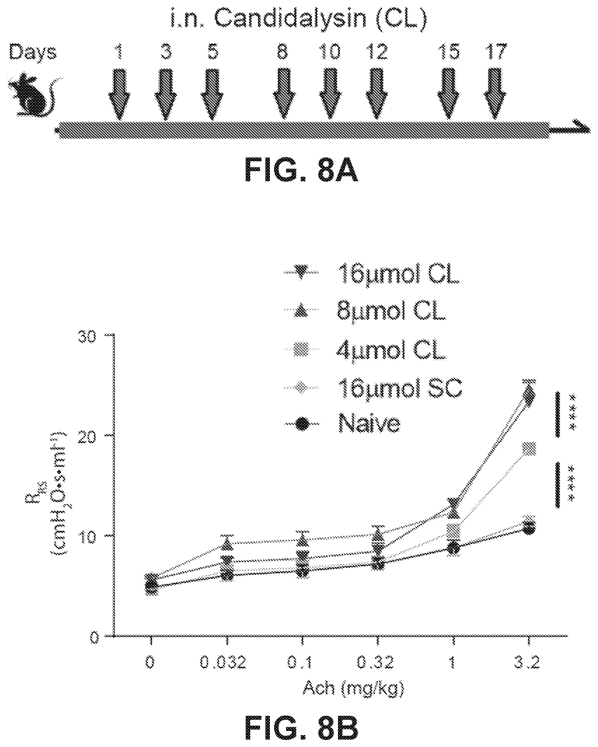

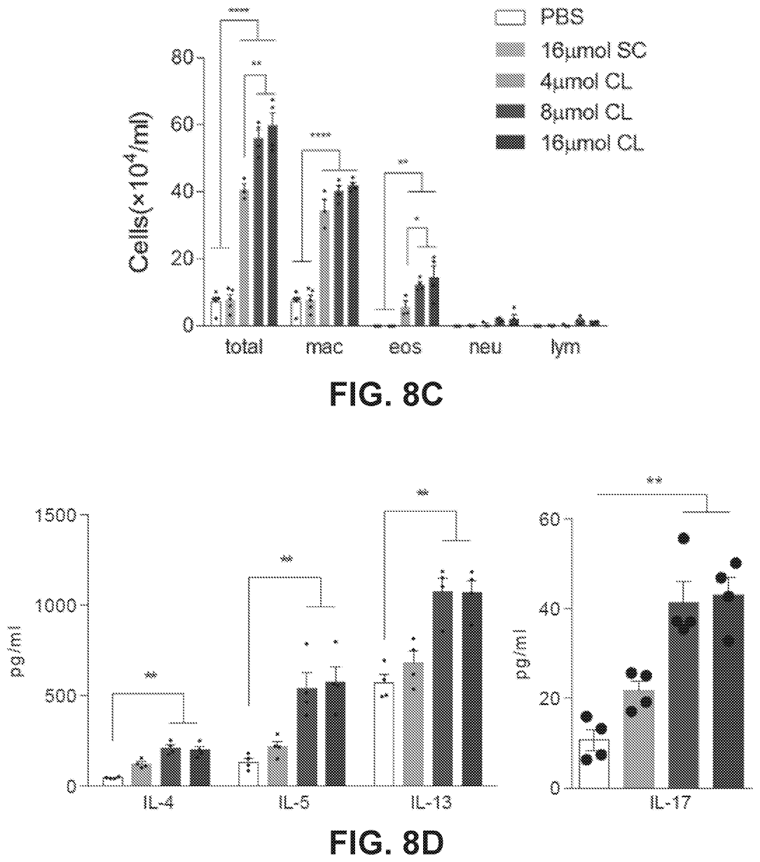

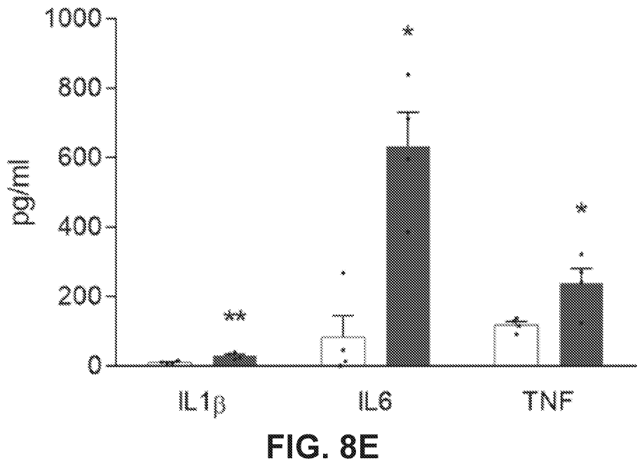

The complete spectrum of allergic airway disease elicited by viable filamentous fungi can be comparably reproduced by representative proteinases that they secrete (Kheradmand et al., 2002). Given the minor contribution of C. albicans proteinases to allergic responses, candidalysin from C. albicans may be alone sufficient to induce allergic airway disease. To test this, mice were challenged intranasally with synthetic, LPS-free candidalysin (CL) or scrambled peptide control (SC) over 17 days using a dose escalating protocol (FIG. 8A) after which the allergic airway disease phenotype was quantified. Candidalysin, but not the scrambled peptide control, induced dose-dependent increases in airway hyper-responsiveness as assessed one day after the last challenge as compared to vehicle challenged mice (FIG. 8B). At the highest dose given (16 μmol), candidalysin also provoked a 600% increase in total BALF inflammatory cells consisting primarily of macrophages, but also eosinophils (FIG. 8C). Th2 cell (IL-4, -5, -13) and Th17 cell (IL-17) cytokines (FIG. 8D) as well as innate pro-inflammatory cytokines (FIG. 8E) were further elicited from whole lung by candidalysin. Thus, analogous to proteinases from filamentous fungi, candidalysin from C. albicans is, in some aspects, sufficient to induce allergic airway disease. Without wishing to be bound by theory, the magnitude of disease induced by exogenously administered candidalysin is reduced compared to that induced by wildtype C. albicans (FIG. 1), potentially because invading fungal hyphae secrete candidalysin into an invasion pocket ENREF 53 in greater concentrations and closer proximity to responsive host cells (e.g., epithelial cells, platelets) than can be achieved with intranasal administration (Moyes et al., 2016).

### Example 3

C. albicans Activates Platelets to Secrete Dkk-1

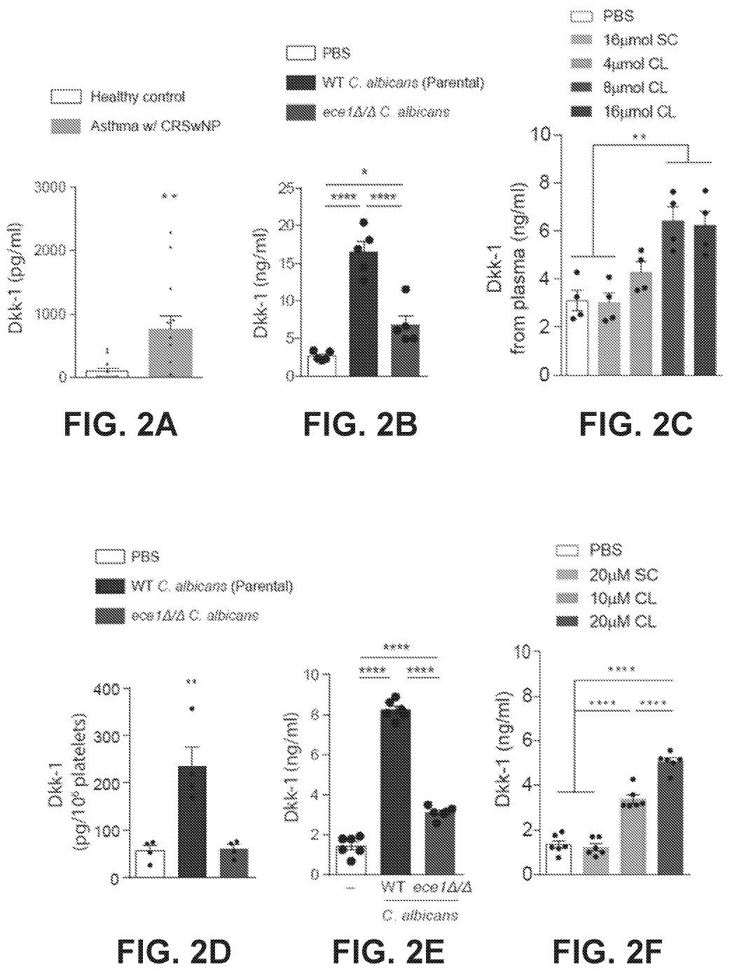

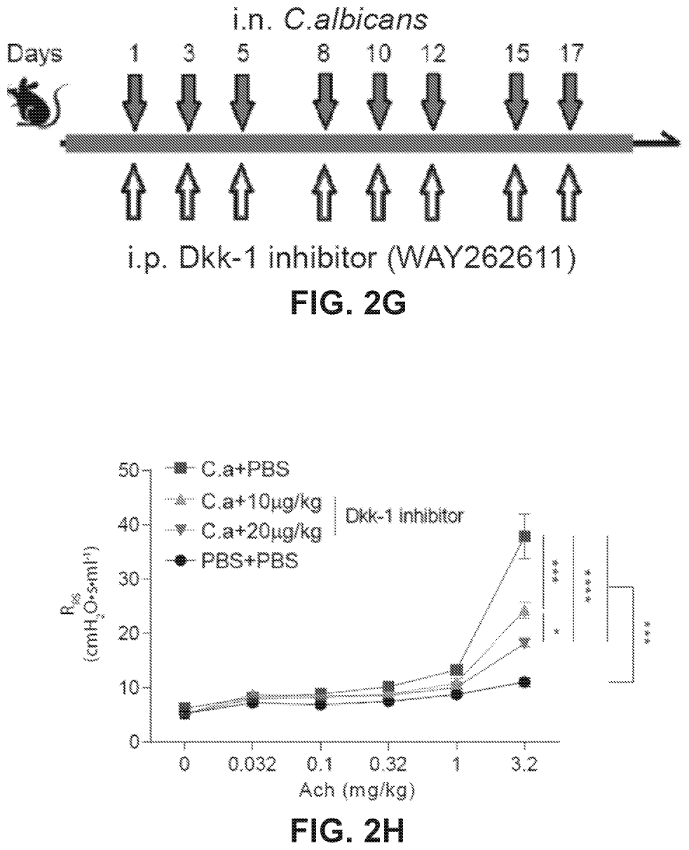

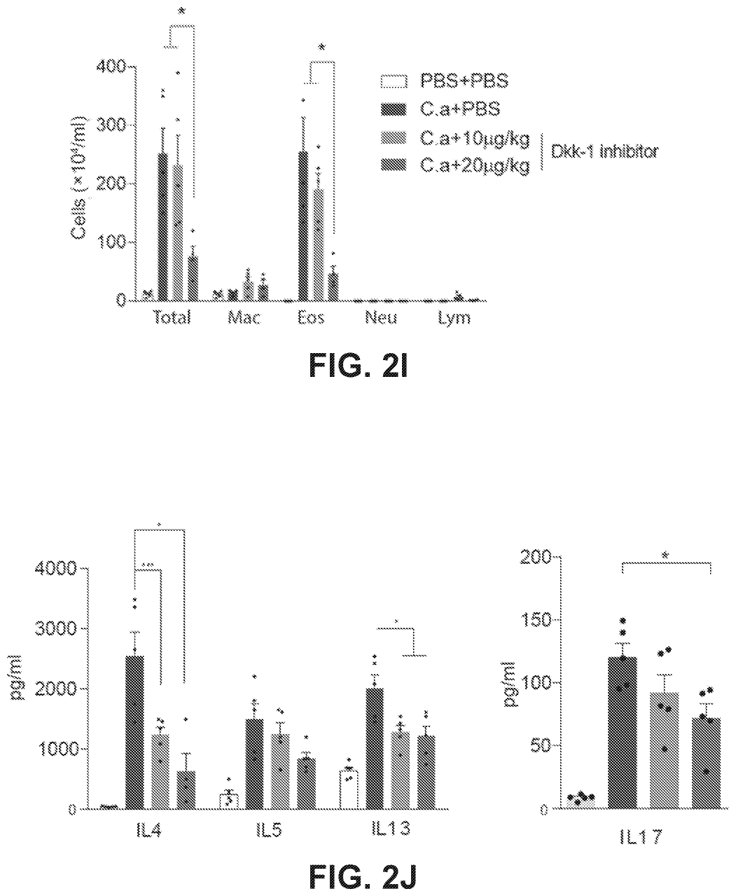

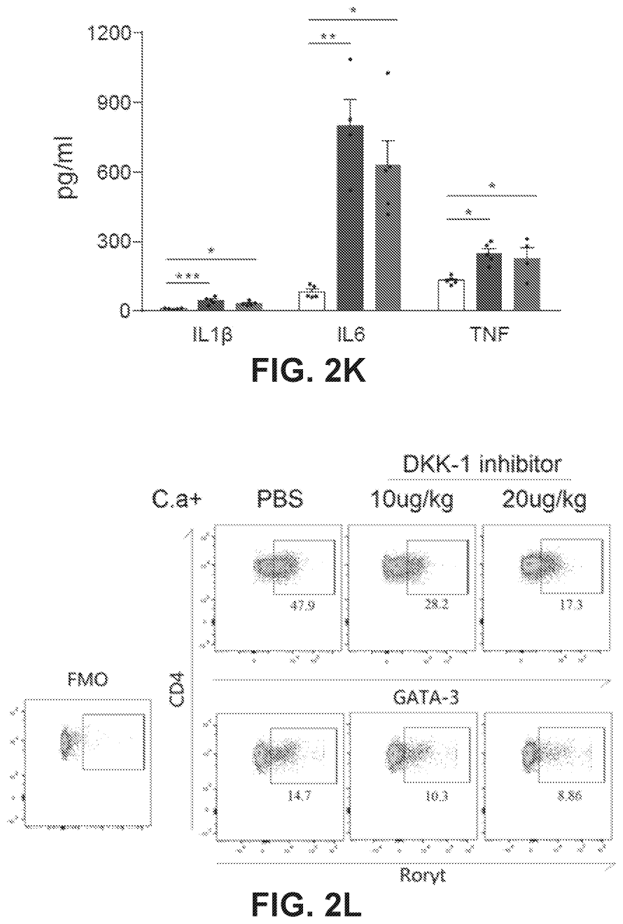

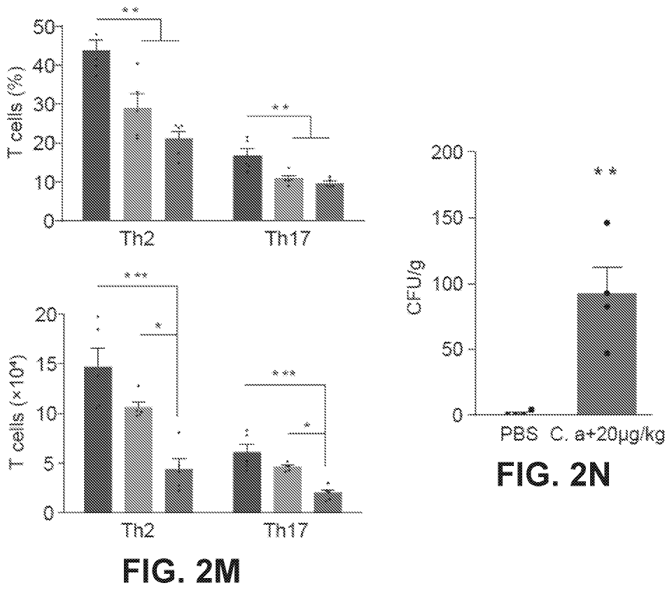

Candidalysin may induce type 2 and type 17 immunity observed in human asthma (Pene et al., 2008; Zhao et al., 2010) and in response to murine airway mycosis (Porter et al., 2011a). The inventors focused on the potential relationship between candidalysin and the Wnt pathway antagonist peptide Dickkopf-1 (Dkk-1) that coordinates chronic type 2 inflammation in response to Leishmania major and dust mite-derived allergens (Chae et al., 2016b). Dkk-1 was quantified in plasma samples from asthma patients with CRS, a patient group that frequently suffers from Candida airway mycosis (Mak et al., 2013; Porter et al., 2014), and Dkk-1 was found to be significantly increased in plasma from asthma and CRS patients compared to control patients with no overt lung or airway disease (FIG. 2A). Similarly, plasma Dkk-1 was found to e elevated approximately five-fold in mice challenged intranasally with wildtype C. albicans as compared to sham-challenged control mice, but only two-fold in sera of ece1Δ/Δ C. albicans-challenged mice (FIG. 2B). In a dose-dependent manner, intranasally administered candidalysin alone also induced significant increases in plasma Dkk-1 (FIG. 2C).

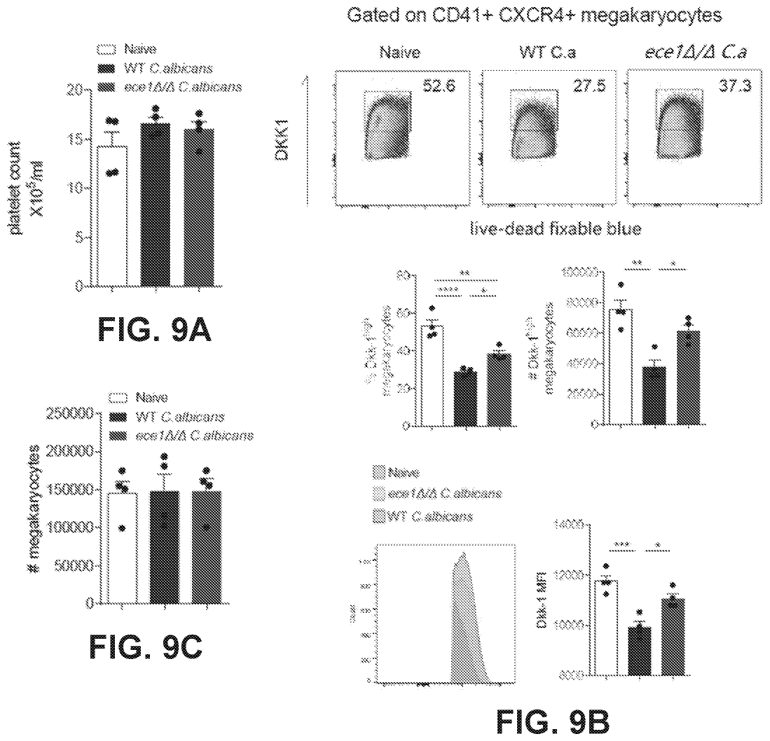

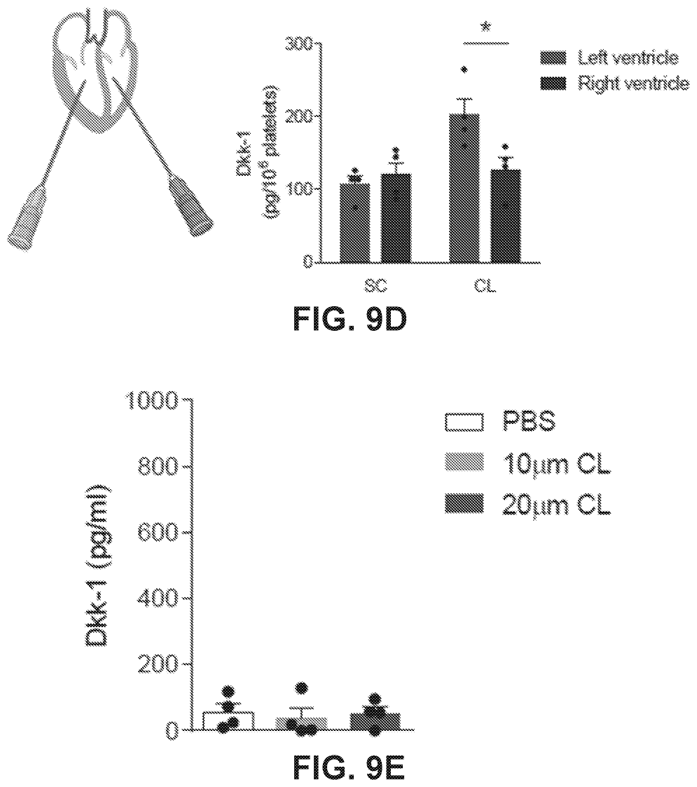

Dkk-1 potentially derives from diverse cellular sources, but may be released primarily from platelets in allergic contexts (Chae et al., 2016b). In support of this, challenge of mice with wildtype, but not ece1Δ/Δ, C. albicans resulted in significantly elevated Dkk-1 in platelets (FIG. 2D) without altering blood platelet counts (FIG. 9A). In contrast, pulmonary CD41+CXCR4+ megakaryocytes (Huang and Cantor, 2009) expressed decreased intracellular Dkk-1 after challenge of mice with wildtype, but not ece1Δ/Δ, C. albicans as assessed by quantifying Dkk1^{high} lung megakaryocytes and Dkk-1 mean fluorescence intensity (MFI) of the same cells (FIG. 9B). Similar to platelets, fungal airway challenge failed to diminish the total number of lung megakaryocytes (FIG. 9C). Thus, Dkk-1 is elevated in both the plasma and platelets of mice with C. albicans-induced allergic airway disease, but is diminished in lung megakaryocytes. These observations suggest that upon fungal challenge, in some aspects, lung megakaryocytes more efficiently shed Dkk-1 through enhanced sequestration in platelets.

To further support the possible pulmonary origin of platelets, wildtype mice were challenged with 16 μmol of candidalysin or scrambled control, and platelets from the left or right ventricles of mice were isolated 2 hours post challenge. Significantly higher Dkk-1 concentrations were found in platelets collected from the left ventricle post-candidalysin challenge (FIG. 9D). Since blood from the left ventricle represents that draining from the pulmonary circulation, these data indicate that, in some aspects, Dkk-1^{high} platelets may originate from pulmonary megakaryocytes.

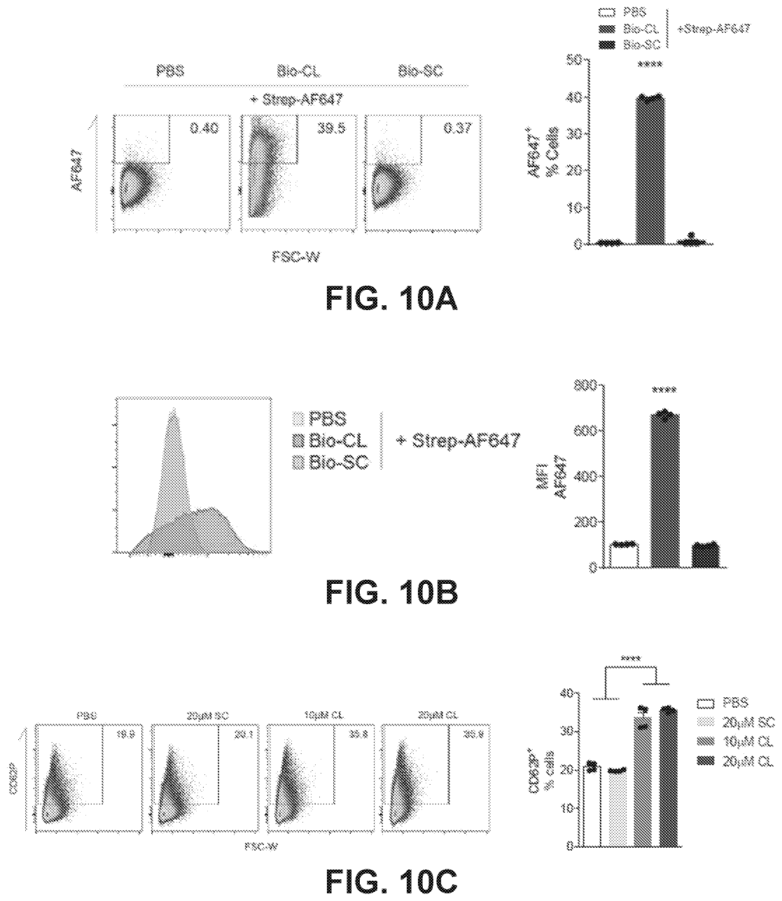

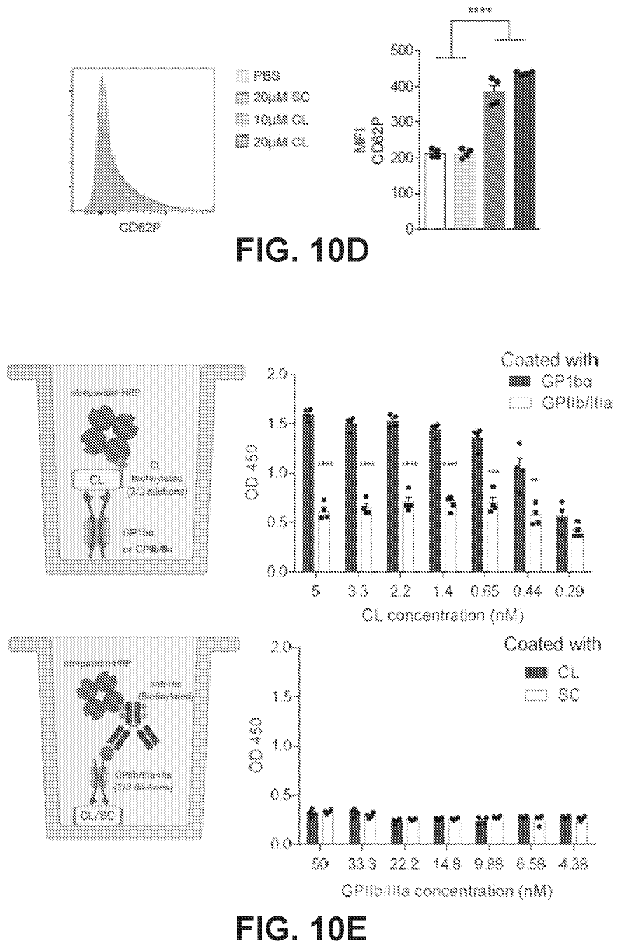

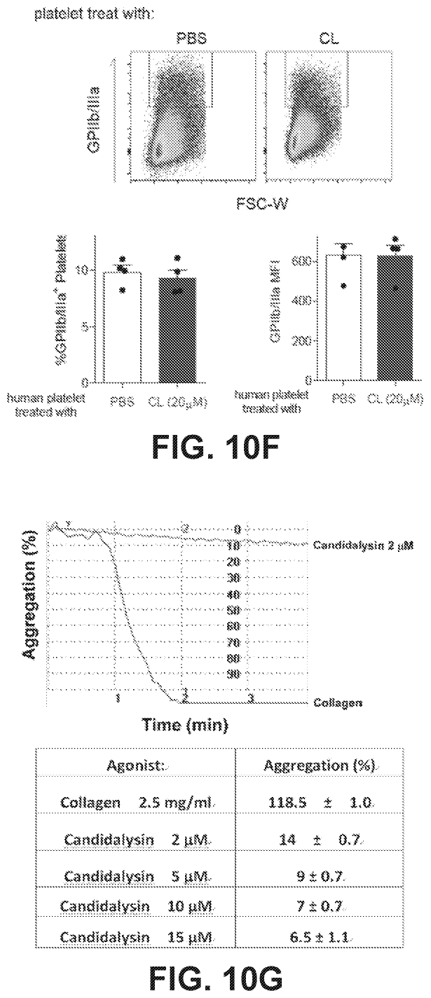

To determine a potential functional relationship between C. albicans, platelets, and Dkk-1, human platelets were incubated with wildtype and ece1Δ/Δ C. albicans in vitro. Wildtype C. albicans cells provoked release of Dkk-1 more than 5-fold above control release amounts, whereas ece1Δ/Δ C. albicans induced significantly less Dkk-1 release (FIG. 2E). Furthermore, candidalysin used at two doses (10 or 20 μM) was alone sufficient to provoke release of Dkk-1 from human platelets (FIG. 2F) (Moyes et al., 2016), but not endothelial cells (FIG. 9E). Candidalysin also acutely activated platelets as assessed by its ability to bind to platelets in plasma and enhance expression of surface CD62P (P-selectin) without inducing lysis (Isenberg et al., 1986) (FIGS. 10A-10D). Together, these results confirm that, in some aspects, candidalysin derived from C. albicans can activate human and mouse platelets to release Dkk-1.

### Example 4

### Dkk-1 Drives C. Albicans-Dependent TH2 and TH17 Cell Responses

The relevance of Dkk-1 to allergic airway disease induced by airway mycosis is unknown. Mice challenged with C. albicans were administered a Dkk-1 inhibitor (WAY 262611) previously shown to block this peptide in vivo (Chae et al., 2016b) (FIG. 2G). In a dose-dependent manner, the Dkk-1 inhibitor progressively reduced airway hyper-responsiveness and BALF inflammatory cell recruitment, most notably reducing BALF eosinophilia (FIGS. 2H-2I).

Dkk-1 inhibition further resulted in the suppression of secretion of type 2 cytokines (IL-4, IL-5, IL-13) from whole lung, but also inhibited secretion of IL-17 (FIG. 2J). Of note, secretion of IL-1β, IL-6 and TNF was not significantly affected by the inhibitor (FIG. 2K). Flow cytometric analysis further revealed the decreased recruitment to whole lung of GATA3⁺Th2 and RORγt⁺ Th17 cells in a manner that correlated with inhibitor dose (FIGS. 2L-2M). Consequently, C. albicans-challenged mice with impaired Th2 and Th17 cell responses due to the Dkk-1 inhibitor had markedly elevated lung fungal burdens (FIG. 2N).

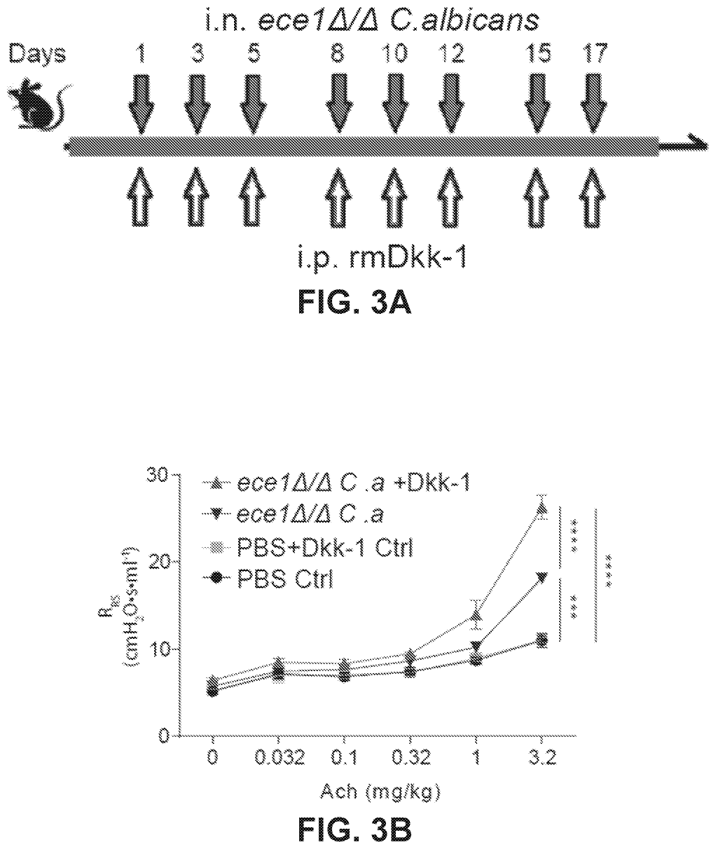

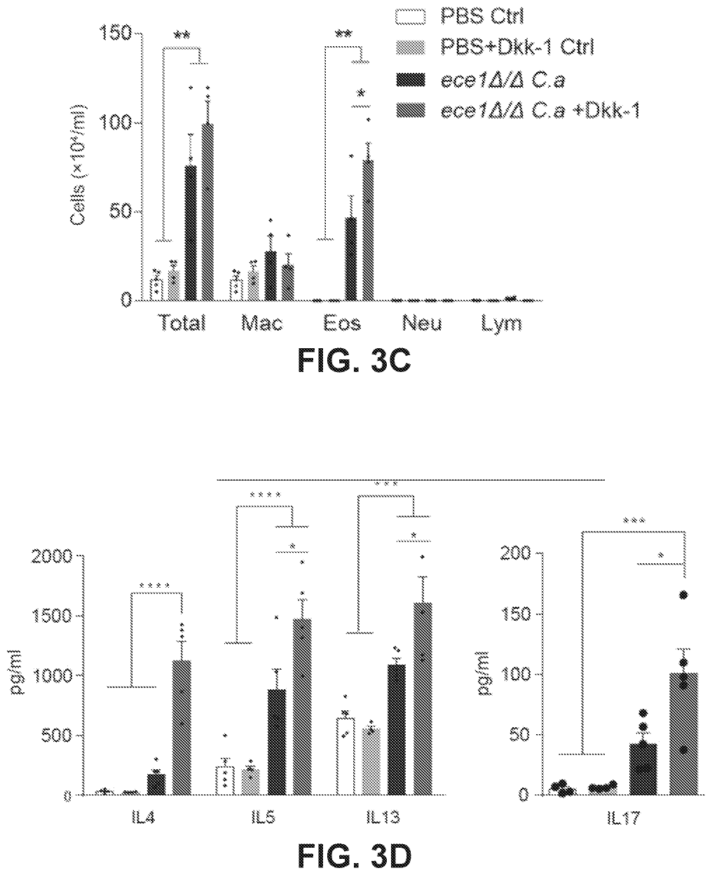

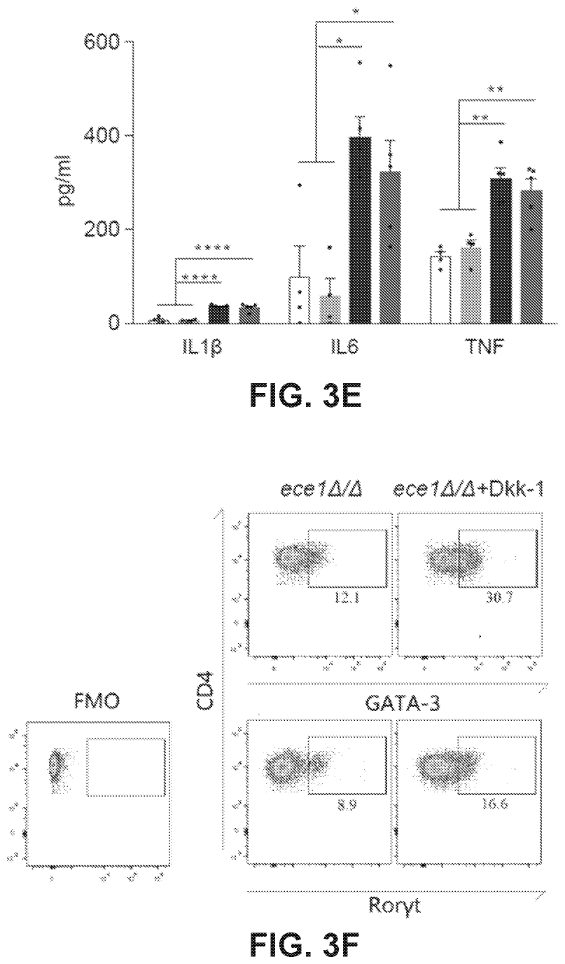

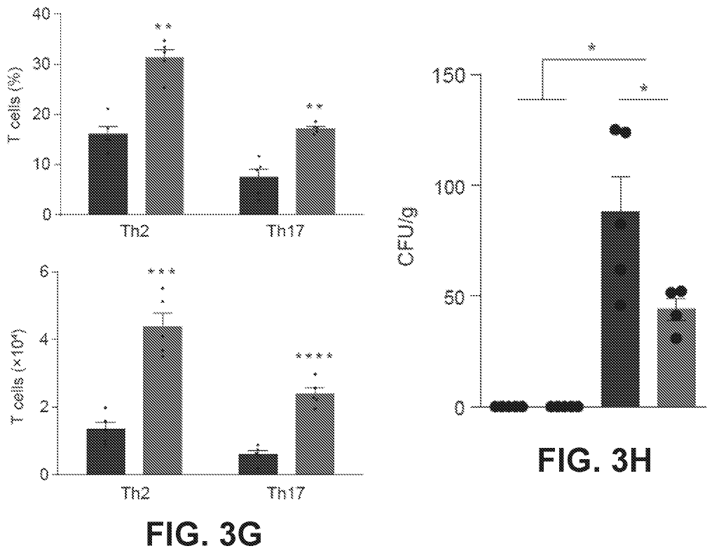

The preceding observations suggest that, in some aspects, candidalysin critically influences the induction of Th2 and Th17 cell responses by stimulating the secretion of Dkk-1. To confirm, synthetic mouse Dkk-1 was administered to mice challenged with ece1Δ/Δ C. albicans (FIG. 3A). Dkk-1, but not control peptide, significantly enhanced airway hyper-responsiveness (FIG. 3B). Consistent with these observations, exogenously administered Dkk-1 markedly enhanced both the recruitment of inflammatory cells, especially eosinophils, to the airways and the secretion of lung Th2 cytokines, especially IL-4, but also IL-17 (FIGS. 3C-3D). Although exogenous Dkk-1 did not influence the secretion of innate pro-inflammatory cytokines (FIG. 3E), it did markedly increase recruitment to lung of GATA3⁺Th2 and RORγt⁺Th17 cells (FIG. 3F-3G). Moreover, fungal lung burdens were significantly reduced after exogenous Dkk-1 treatment (FIG. 3H). Dkk-1 alone had no effect on any index of allergic airway disease when administered through the intraperitoneal route (data not shown). Thus, in some aspects, Dkk-1 may be an important mediator of lung Th2 cell responses (Chae et al., 2016b), and may be equally important for the generation of Th17 cell responses in the context of airway mycosis. These findings further confirm that Dkk-1 is essential for the control of C. albicans growth in the lung.

### Example 5

Mechanism of Dkk-1 Release from Platelets

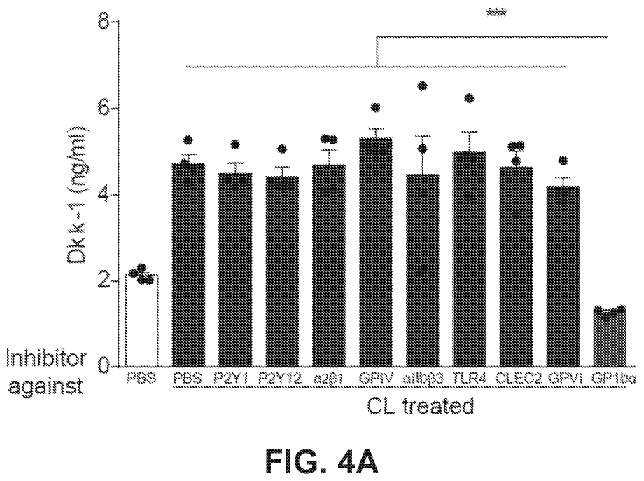

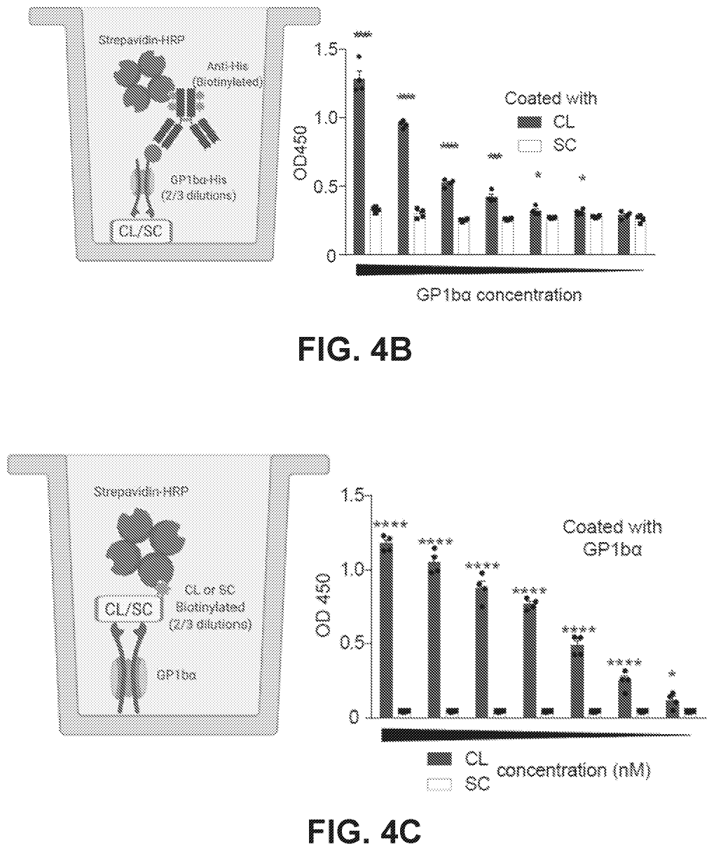

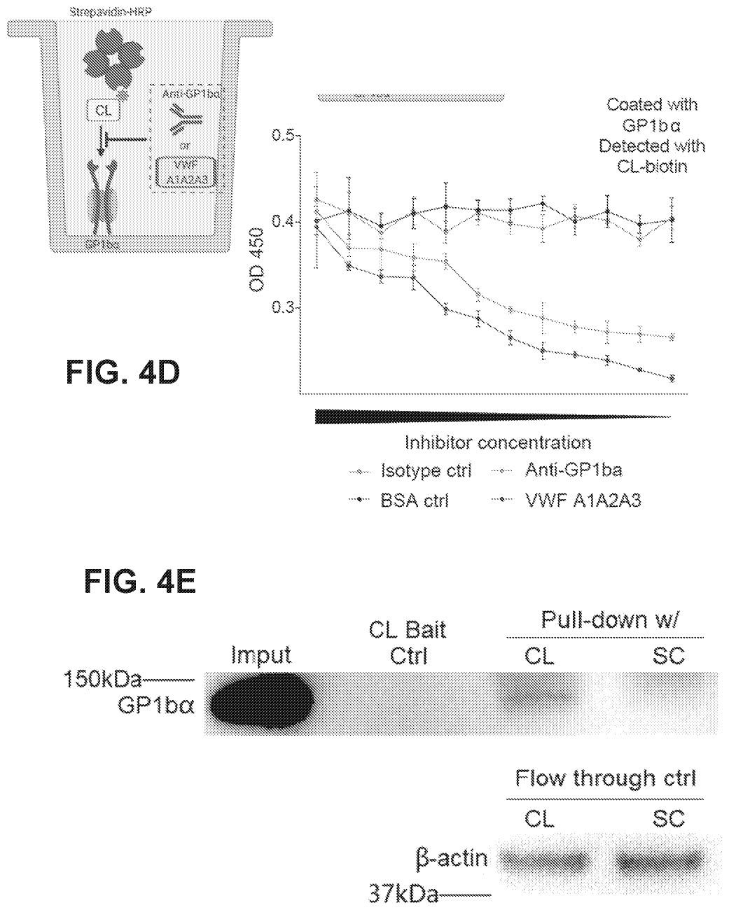

Although candidalysin acutely induces lysis of host cells at concentrations above 20 μM, suggesting a non-specific mechanism for Dkk-1 release (Moyes et al., 2016), platelet activation and Dkk-1 release at candidalysin concentrations of 10 and 20 μM suggested that candidalysin potentially activates a specific platelet receptor (FIG. 2F; FIGS. 10A-10D). To screen for a specific candidalysin receptor, human platelets were incubated with candidalysin after previous addition of blocking antibodies to the receptors P2Y1, P2Y12, α2β1, GPIV, α_{IIb}β₃, TLR4, CLEC2, GPVI and GP1bα after which Dkk-1 release was measured. Only GP1bα blockade inhibited candidalysin-dependent Dkk-1 release. These findings indicated that, in some aspects, candidalysin-dependent Dkk-1 release occurs through a specific mechanism, and GP1bα may be a candidate candidalysin receptor (FIG. 4A).

To further establish the potential importance of GP1bα as a mammalian candidalysin receptor, direct binding between GP1bα and candidalysin was demonstrated using a modified ELISA in which either candidalysin or GP1bα was used as the capture reagent and His-tagged GP1bα or biotinylated candidalysin were used, respectively, as the second reagent (FIGS. 4B-4C). Regardless of the configuration, these assays consistently demonstrated concentration-dependent binding between candidalysin and GP1bα (FIGS. 4B-4C) that was inhibitable by either a blocking anti-GP1bα antibody or VWF A1A2A3 tridomain, the latter representing a fragment of VWF that binds GP1bα through the A1 domain (FIG. 4D) (Azuma et al., 1991). To further demonstrate the interaction between candidalysin and GP1bα, pull-down assays using human platelet lysates were performed. Biotinylated candidalysin, but not its scrambled control peptide, was found to physically associate with GP1bα from human platelet lysates as determined by western blotting (FIG. 4E). Collectively, these observations confirm that, in some aspects, GP1bα on platelets specifically binds to candidalysin.

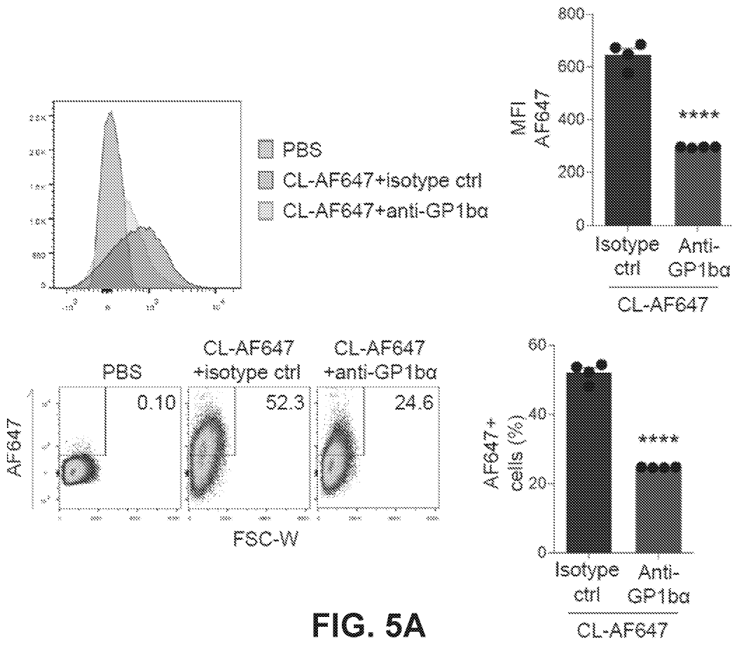

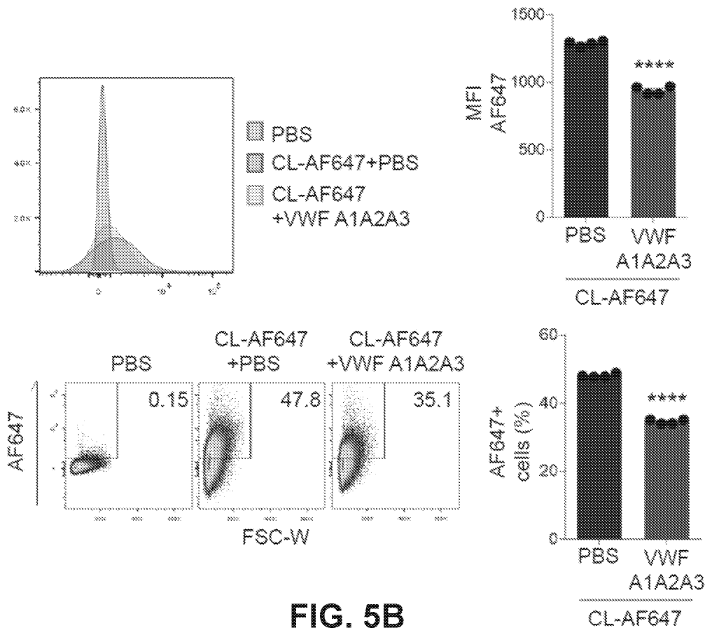

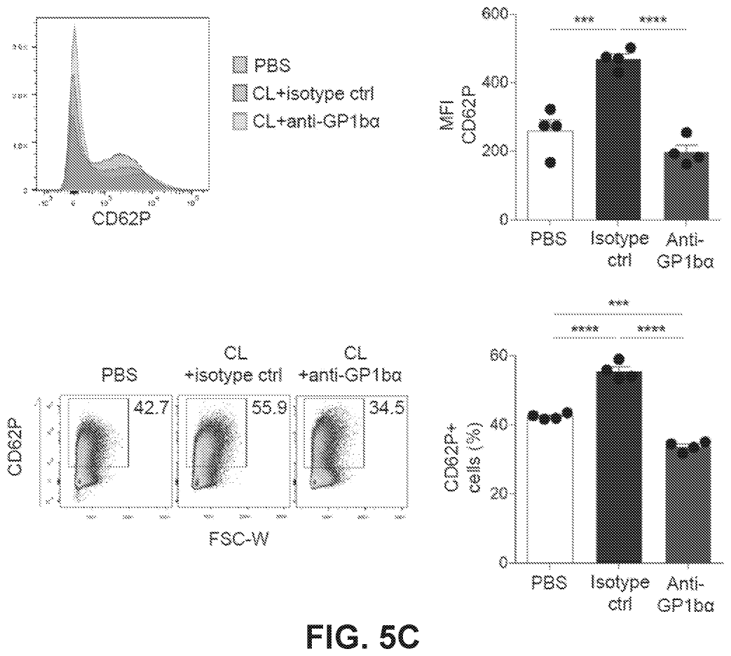

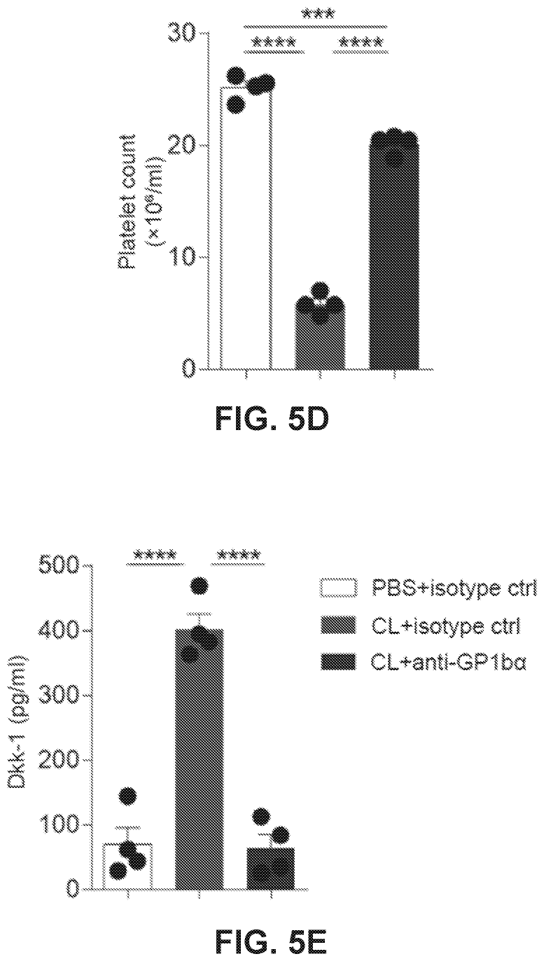

Additional assays were conducted using human platelets to further address the interaction between candidalysin and GP1bα. As assessed by flow cytometry, fluorescently labeled candidalysin readily bound to human platelets in the presence of plasma, but this interaction was blocked approximately 50% by pre-treating platelets with a non-activating antibody against GP1bα (FIG. 5A, FIGS. 10A-10D). GP1bα further blocked with VWF A1A2A3 tridomain also significantly reduced binding of candidalysin to platelets (FIG. 5B).

The preceding results further demonstrate that candidalysin interacts specifically with GP1bα, but in addition suggest that candidalysin can also non-specifically interact with platelets, e.g., by binding to additional receptors or intercalating within the platelet membrane. Further analyses were therefore conducted to determine if another platelet receptor, GPIIb/IIIa, which is critical for platelet aggregation and thrombosis (Calvete, 1995; Shattil, 1999), could interact with candidalysin. Using a similar binding assay, the inventors were unable to demonstrate any direct affinity between candidalysin and GPIIb/IIIa (FIG. 10E). Moreover, GPIIb/IIIa expression was unaltered on human platelets stimulated with candidalysin (FIG. 10F). Candidalysin, in marked contrast to the activating GPIIb/IIIa ligand collagen, failed to induce platelet aggregation in vitro (FIG. 10G).

Despite the non-specific binding of candidalysin to platelets, GP1bα blockade completely inhibited candidalysin-dependent activation of platelets prepared in plasma as assessed by CD62P upregulation (FIG. 5C). Anti-GP1bα further suppressed CD62P expression lower than that observed in naïve platelets, indicating that GP1bα can integrate a variety of inputs to mediate platelet activation. A more complex mechanism whereby candidalysin interacts with plasma VWF to co-activate platelets was considered. However, washed platelets that were prepared free of plasma VWF remained highly susceptible to candidalysin-dependent activation as assessed by Dkk-1 release and autolysis (FIGS. 5D-5E). Thus, candidalysin interacts with platelets in multiple ways, but only the specific interaction with GP1bα in the absence of VWF is sufficient to mediate platelet activation and Dkk-1 release. Critically, such activation occurs without eliciting platelet aggregation.

### Example 6

### Hemostatic Role of Platelets During Airway Mycosis

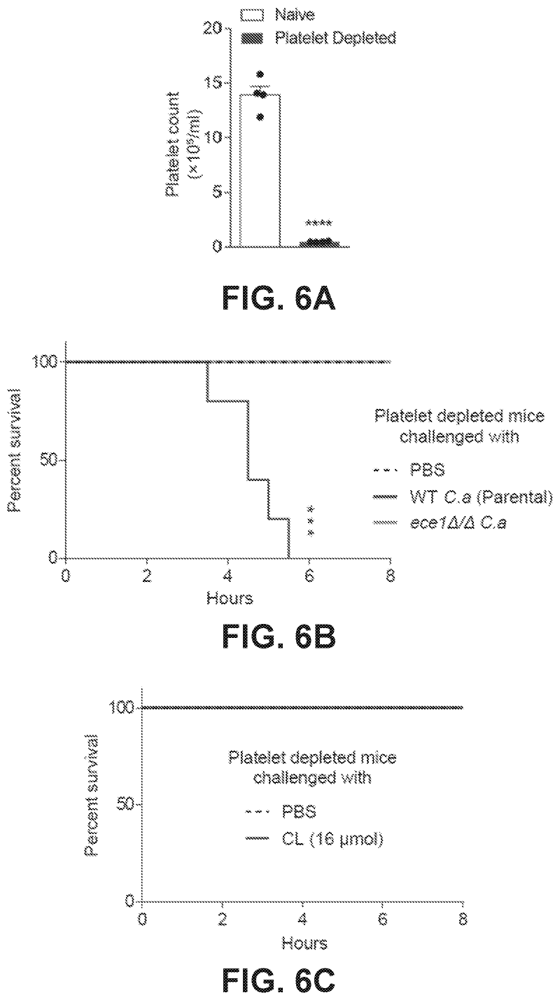

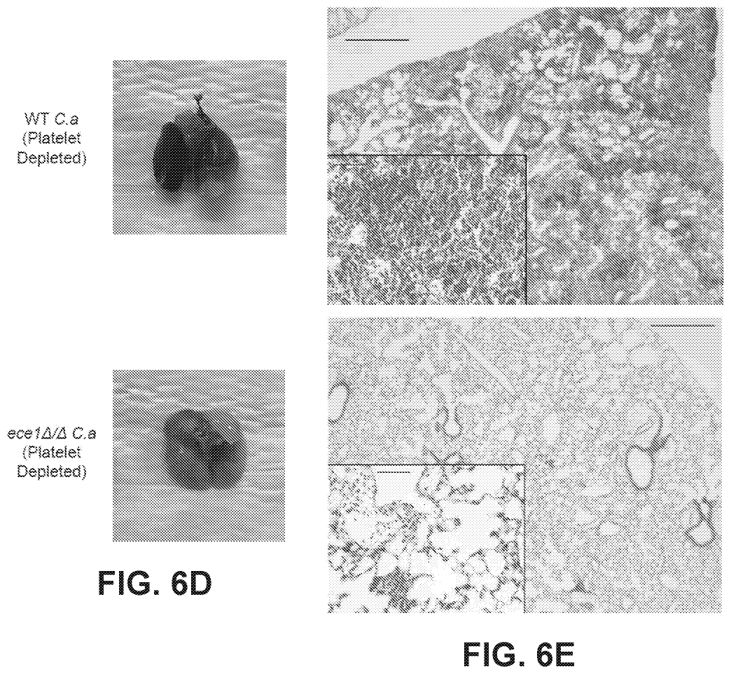

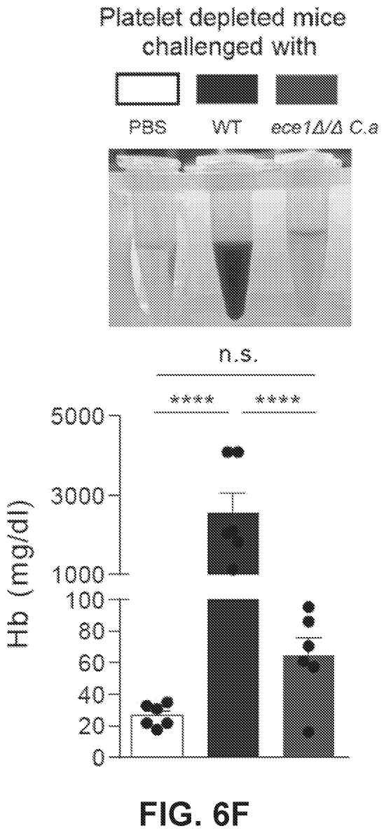

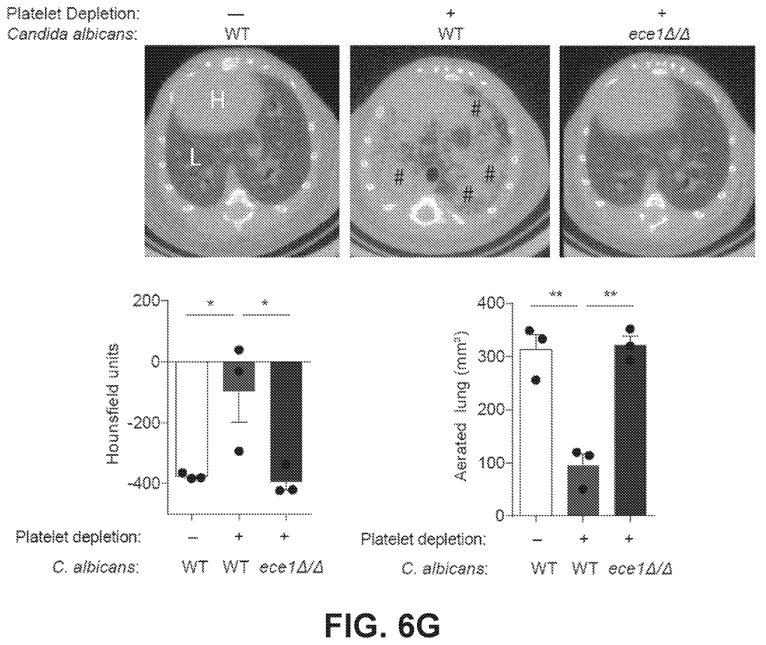

To confirm the importance of platelets to C. albicans-dependent allergic airway disease, administered 10⁵ C. albicans cells were intranasally administered to mice depleted >95% of platelets (FIG. 6A). All platelet-depleted mice succumbed to this infectious challenge within 6 h after fungal exposure, whereas ece1Δ/Δ C. albicans cells induced no mortality or apparent illness (FIG. 6B). Additionally, wildtype, platelet-replete mice experienced no mortality or illness after a single challenge with up to 10⁷ viable cells of C. albicans (data not shown).

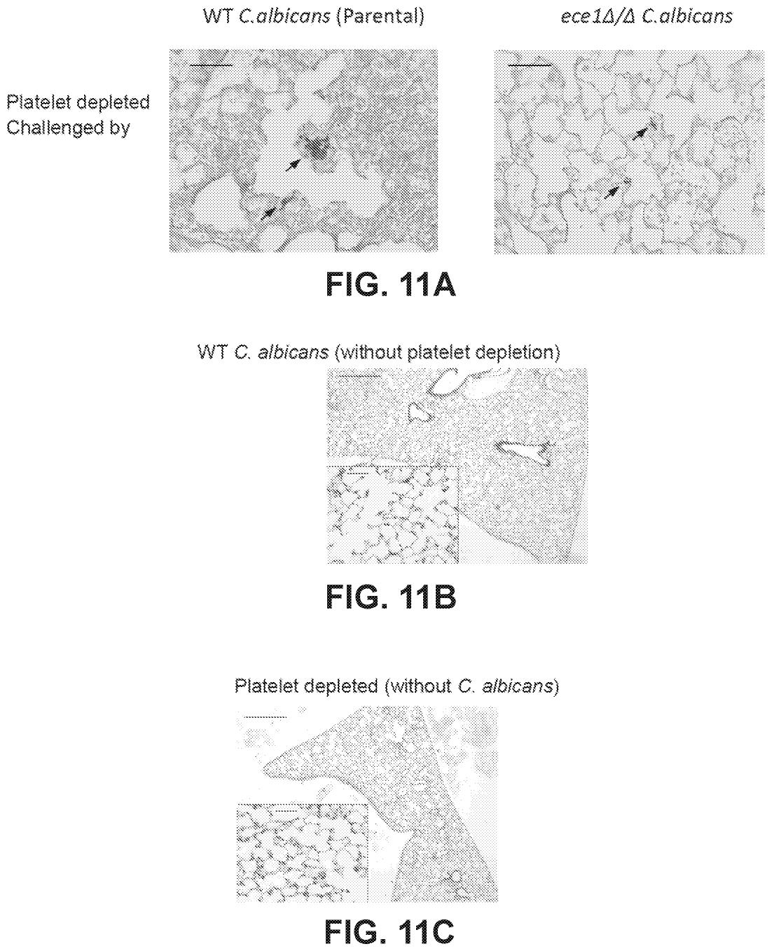

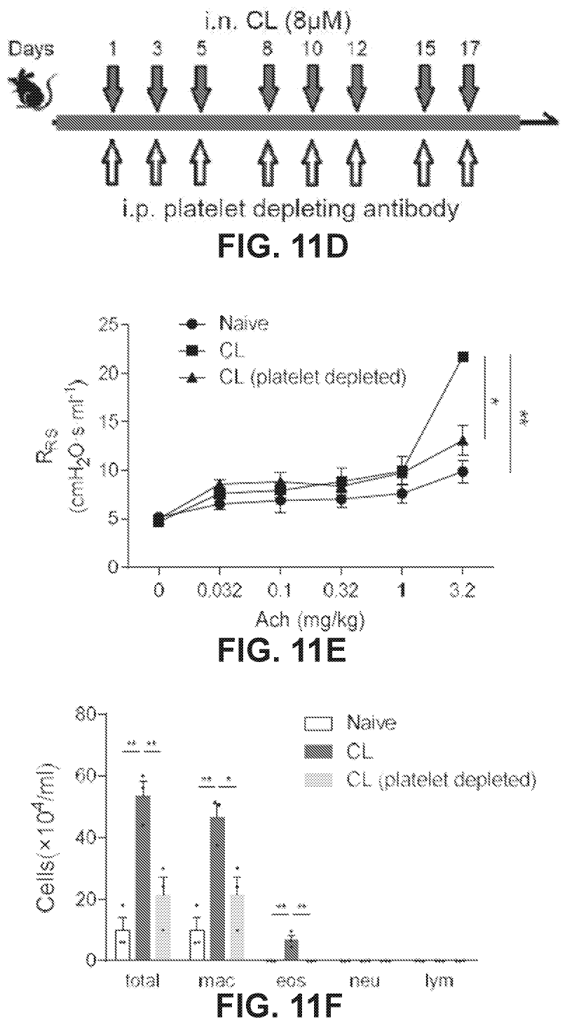

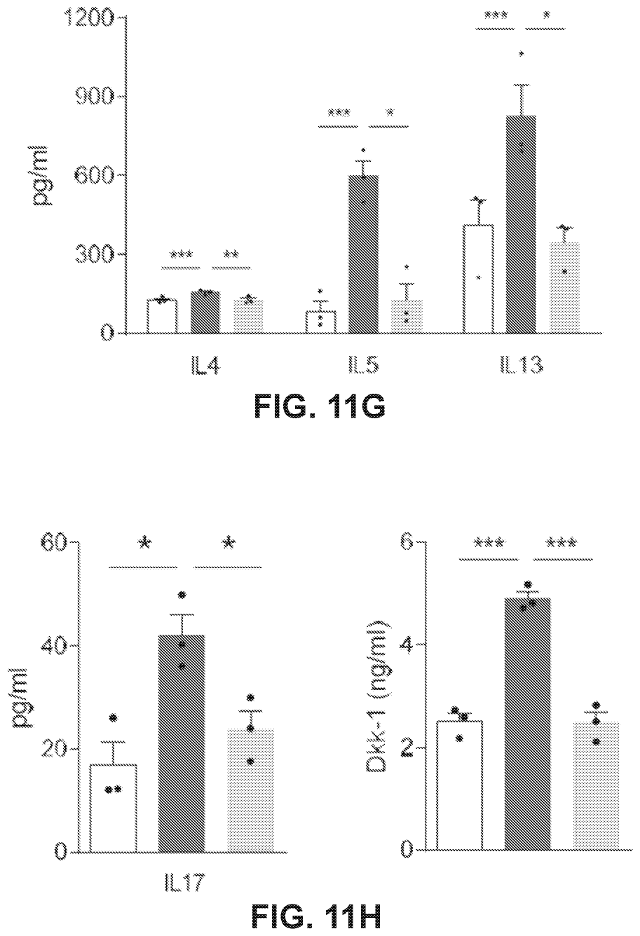

In contrast, a single challenge of recombinant candidalysin peptide alone caused no mortality or apparent illness when introduced into the airways of platelet-depleted mice (FIG. 6C), suggesting that, in some aspects, the lethal outcomes with viable yeast cells reflected additional requirements such as growing hyphae that are able to extend beyond the airway epithelium and rupture capillary beds. Examination of deceased animals revealed apparent pulmonary hemorrhage involving most of the lung that was confirmed by histologic analysis (FIGS. 6D-6E) that also revealed fungal hyphae within terminal airways and alveoli (FIG. 11A). Bronchoalveolar lavage further retrieved gross blood from mouse lungs that contained abundant hemoglobin (FIG. 6F). MicroCT analyses demonstrated that the predominantly normal lung seen in platelet-sufficient, wildtype C. albicans-challenged mice and platelet-depleted mice receiving ece1Δ/Δ C. albicans was largely replaced by an X-ray-dense material, most likely representing blood, that strongly enhanced lung density (measured in Hounsfield units) and attenuated aerated lung volume (FIG. 6G). Of note, platelet depletion or C. albicans challenge individually induced no pulmonary hemorrhage (FIGS. 11B-11C). Thus, in addition to driving type 2 and type 17 immunity through Dkk-1, these findings demonstrate that platelets may play an indispensable hemostatic role during acute airway infection with C. albicans.

### Example 7

### Role of Platelets in Candidalysin-Dependent Dkk-1 Release and Allergic Airway Disease

While important in revealing the critical hemostatic role of platelets during C. albicans airway mycosis, the preceding experiment precluded determining if platelets are also important sources of Dkk-1 that drives type 2 and type 17 responses in this context. Thus, normal and platelet-depleted mice were challenged intranasally with synthetic candidalysin, which alone is sufficient to induce allergic airway disease (FIG. 8). As expected, platelet-replete mice developed significant airway hyperresponsiveness, airway eosinophilia, and type 2 and type 17 cytokine responses in comparison to sham-challenged animals (FIGS. 11D-11G). In contrast, mice with sustained depletion of platelets demonstrated no significant inflammatory response to candidalysin and in fact were not significantly different from vehicle-challenged mice for all measured parameters. However, thrombocytopenic mice did manifest epistaxis and one died in response to candidalysin challenge (data not shown). Candidalysin induced more than a 2-fold increase in plasma Dkk-1 in platelet sham-depleted mice but had no effect on plasma Dkk-1 in mice almost entirely deficient in circulating platelets (FIG. 11H).

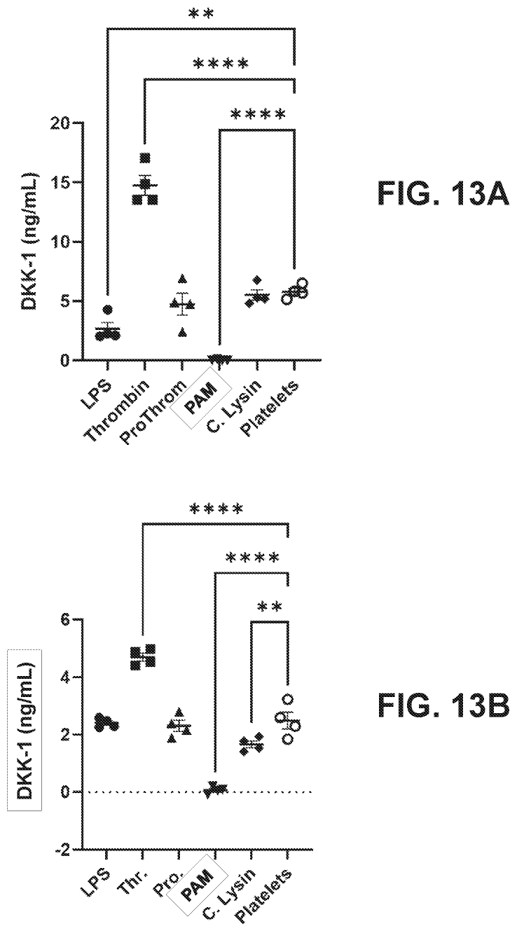

To investigate agents driving Dkk-1 release by platelets, human platelets were stimulated with lipopolysaccharide (LPS), thrombin, prothrombin (ProThrom), proteinase of Aspergillus melleus (PAM), or candidalysin (C. Lysin) overnight at 4° C. (FIG. 13A) or 37° C. (FIG. 13B). Unstimulated human platelets served as a control (platelets). Following stimulation, Dkk-1 concentration in the supernatant was quantified. Stimulation with thrombin significantly increased Dkk-1 release by platelets at both 4° C. and 37° C. Stimulation with PAM alone did not increase Dkk-1 release, but PAM converts prothrombin to thrombin, and therefore, in some aspects, PAM is expected to indirectly result in an increase in Dkk-1 release.

These findings demonstrate that platelets are a source of Dkk-1 release in response to candidalysin challenge of the mouse lung and that platelets can be important to both the generation of Th2 and Th17 cell responses and allergic airway disease in this context. Additionally, fungal proteinases are indirectly linked to Dkk-1 release.

### Example 8

### Fungal Experimental Allergic Airway Disease is Steroid Resistant

Standard treatment for severe allergic airway disease (AAD) (e.g., asthma) is corticosteroids (e.g., prednisone, fluticasone propionate, dexamethasone), which are very powerful anti-inflammatories. Thus, steroid-resistant AAD (e.g., asthma) is especially concerning.

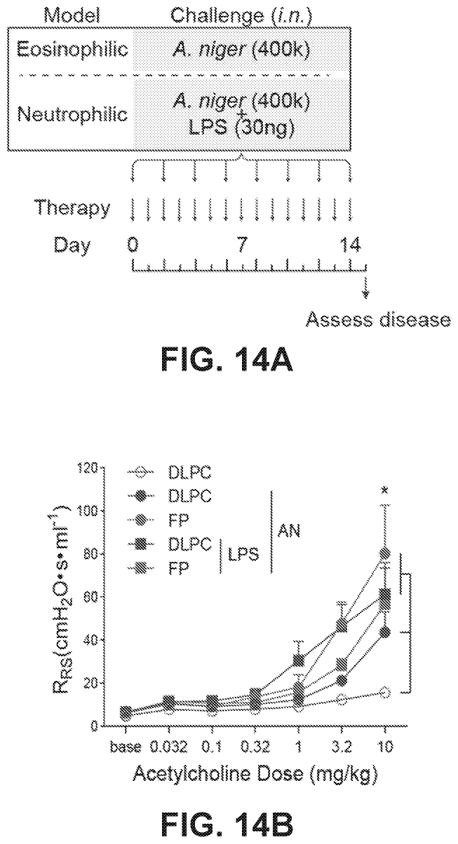

As shown in the severe eosinophilic and neutrophilic steroid-resistant AAD models of FIGS. 14A-14B, mice challenged with Aspergillus niger (AN) develop an asthma phenotype, here showing airway hyperresponsiveness (AHR), that is steroid (fluticasone propionate; FP, a commonly used steroid in asthma) resistant. Illustrated in FIG. 14A is an experimental timeline for assessing the effect of daily FP or vehicle (liposome: dilauroylphosphatidylcholine: DLPC) treatment on AHR. AHR was measured as a function of respiratory system resistance (R_{RS}(cmH₂O·s·ml⁻¹)) in C57BL/6 mice challenged intranasally (q.o.d.) with 4×10⁵ A. niger (AN) conidia ±30 ng lipopolysaccharide (LPS) (FIG. 14B). FIG. 14B shows that treatment of AAD with FP can in fact exacerbate airway hyperresponsiveness.

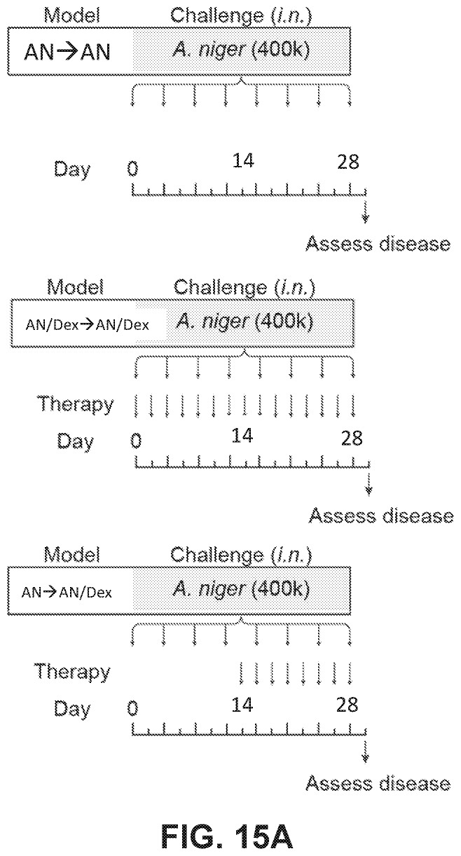

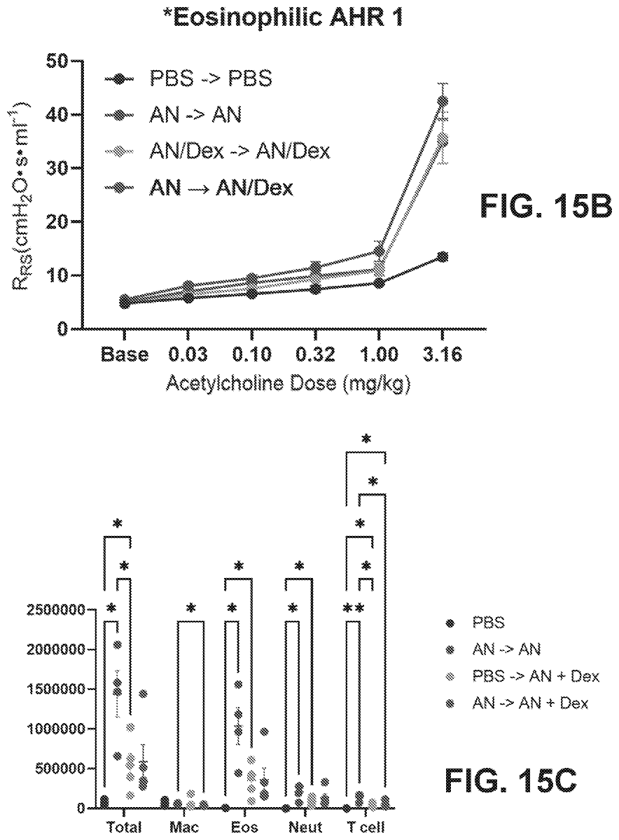

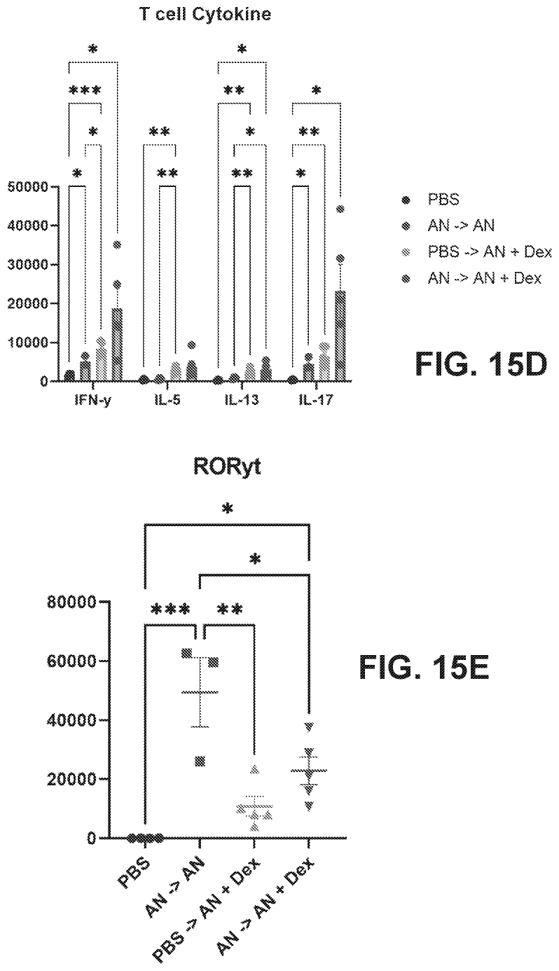

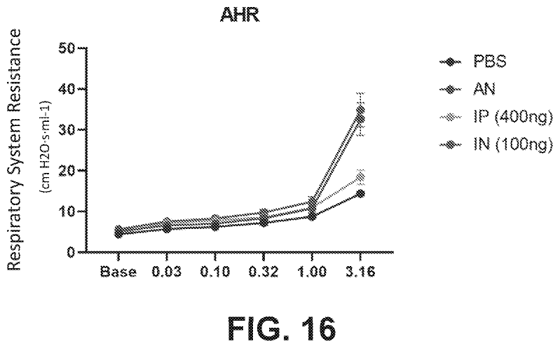

Similarly, as shown in the severe eosinophilic steroid-resistant AAD model of FIGS. 15A-15E, mice challenged with Aspergillus niger (AN) develop an asthma phenotype, here showing airway hyperresponsiveness, that is also resistant to dexamethasone (Dex), a steroid that is more potent than FP. Illustrated in FIG. 15A are experimental timelines for assessing the effect of Dex treatment on airway hyperresponsiveness in an eosinophilic steroid-resistant AAD model. C57BL/6 mice were treated over 28 days as follows: PBS (control) daily (PBS→PBS); intranasal challenge (q.o.d.) with 4×10⁵ A. niger (AN) conidia every other day (AN→AN; FIG. 15A, top); intranasal challenge (q.o.d.) with 4×10⁵ A. niger (AN) conidia every other day+Dex daily (AN/Dex→AN/Dex; FIG. 15A, middle); intranasal challenge (q.o.d.) with 4×10⁵ A. niger (AN) conidia every other day+Dex daily on days 14-28 (AN→AN/Dex; FIG. 15A, bottom). The effect of the various treatments was measured as a function of respiratory system resistance (R_{RS}(cmH₂O·s·ml⁻¹)) (FIG. 15B), immune cell response (FIG. 15C), cytokine release (FIG. 15D), and Th17 (RORγt positive) cell response (FIG. 15E) in C57BL/6 mice. These data show that even the extremely potent steroid dexamethasone fails to reduce airway hyperresponsiveness, despite reductions in the level of some inflammatory cells.

### Example 9

### Dkk-1 Inhibition can Treat Diverse Types of Fungal Asthma

C57BL/6 mice (4-6 weeks old) were challenged every second day with 400,000 viable conidia of AN while at the same time receiving a Dkk-1 antagonist (WAY262611) intranasally (IN) or intraperitoneally (IP) for 14 days. Airway hyperresponsiveness as assessed by enhanced increases in respiratory system resistance in response to increasing doses of acetylcholine chloride injected intravenously were determined as compared to PBS control mice (blue line). The data demonstrate that antagonism of Dkk-1 systemically (via the IP route; green line), but not locally (via the IN route; purple line), abrogates AHR induced by AN alone (red line). Thus, regardless of the fungus used, either Candida albicans or A. niger as shown herein, Dkk-1 can induce AHR. Statistics: IP versus IN or AN lines: P<0.05.

### Example 10

### Exemplary Materials and Methods

### A. Animals

8 week-old C57BL/6J male and female mice (wildtype and Tlr4^{-/-}) were purchased from Jackson Laboratories. All mice were bred and housed at an American Association for Accreditation of Laboratory Animal Care-accredited vivarium under specific-pathogen-free conditions. All experimental protocols were approved and followed federal guidelines.

### B. Human Plasma Samples

De-identified human plasma samples were obtained from healthy control subjects with no allergic airway diseases or those diagnosed with asthma and CRS. Samples were originally obtained after obtaining informed consent under IRBs.

### C. Fungi and Reagents

For experiments in which only wildtype fungus was used, wildtype C. albicans was isolated as described previously (Wu et al., 2019) and validated as C. albicans. SAP gene deletant, ece1Δ/Δ (ECE1 gene encodes the Ecelp protein from which candidalysin is derived), and their parental wildtype strain (BWP17/CIp30) of C. albicans were generated as previously described (Hube et al., 1997; Moyes et al., 2016; Sanglard et al., 1997). C. albicans was propagated in YPD broth overnight at room temperature and collected in pyrogen-free phosphate buffered saline (PBS; Corning cellgro, Mediatech, Manassas, VA), passed through 40 μm nylon mesh, and washed twice with PBS by centrifugation (10,000 g, 5 min, 4° C.). Fungal cells were then suspended in PBS and aliquots frozen in liquid nitrogen at 5×10⁷/mL. Viability after freezing (>95%) was confirmed by comparing haemacytometer-derived cell counts to CFU as determined by plating serial dilutions on Sabouraud's agar. Thawed, >95% viable cells were washed once, counted, and suspended in normal saline at indicated concentrations for intranasal challenge.

### D. Candidalysin

Synthetic, biotinylated and ALEXA FLUOR® 647-labeled candidalysin or scrambled peptide control were obtained commercially and were >98% pure (Peptide Protein Research Ltd, SO32 1QD, UK, or Genscript 08854, NJ, USA). Candidalysin sequence: SIIGIIMGILGNIPQVIQIIMSIVKAFKGNK. Scrambled control (SC) peptide sequence: IFKIIISKIQIVMGLNGIPIKVAGSQNIGMI. Peptide biotinylation was performed on the N-terminal.

### E. Fibrinogen Cleavage Products (FCPs)

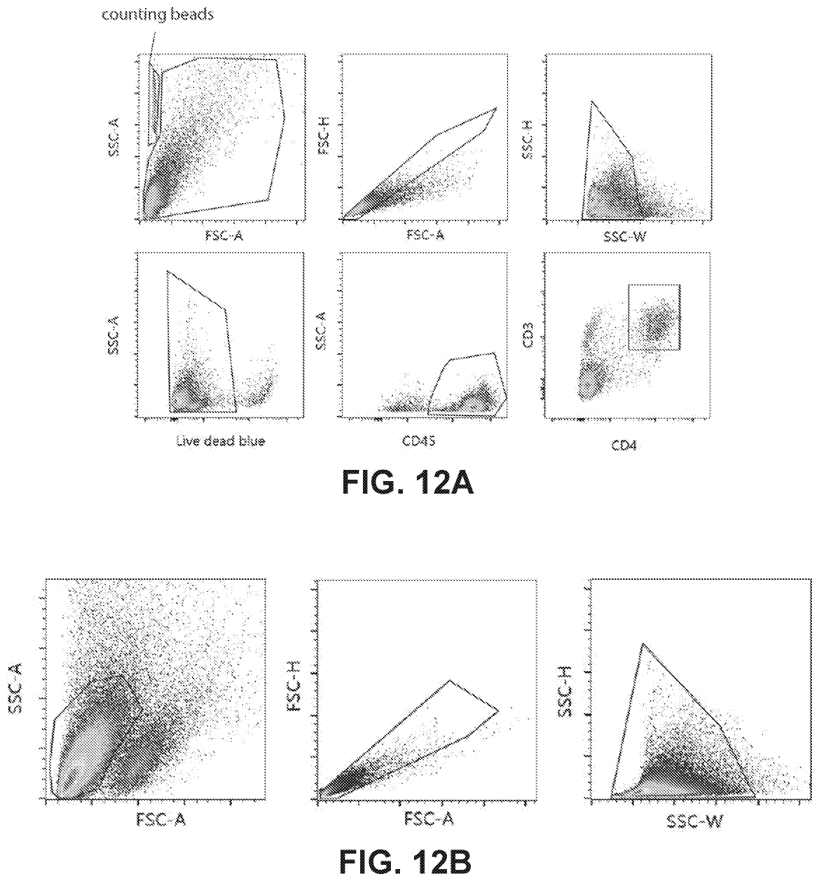

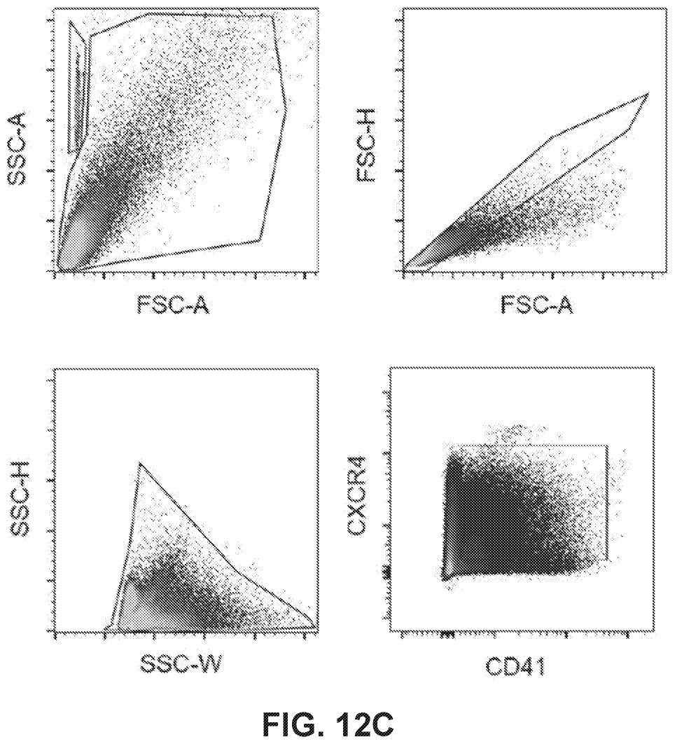

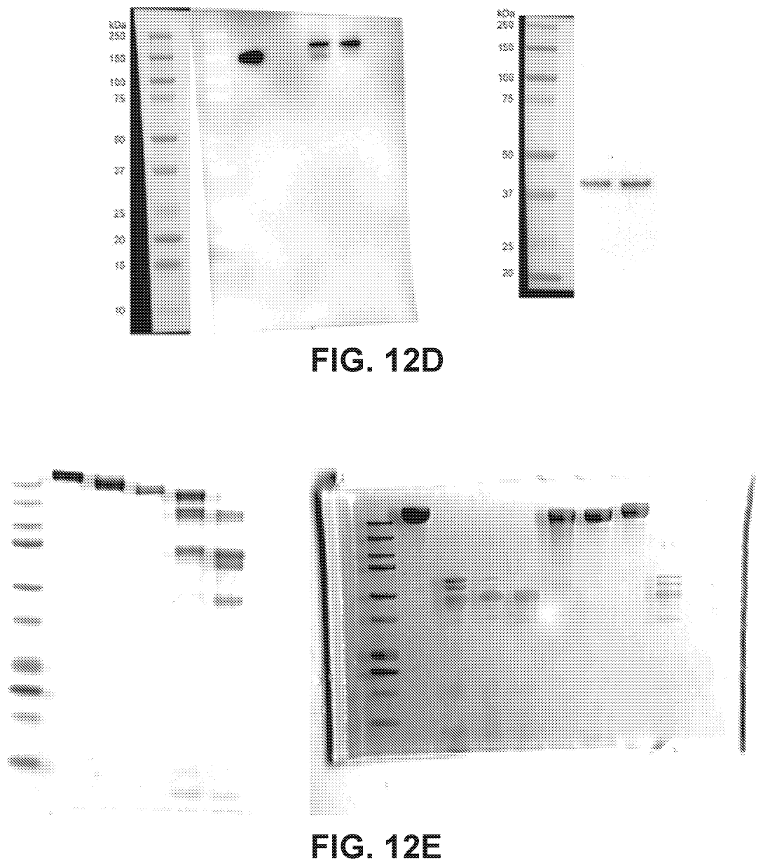

FCPs were prepared by suspending human fibrinogen (HCl-0150R; Haematologic Technologies, Essex Junction, Vermont) at 5 mg/mL in PBS. Proteinase from Aspergillus melleus (PAM; P4032; Sigma-Aldrich, St. Louis, MO), was added to the fibrinogen solution at a concentration of 6 μg/mL for 2 or 6 hours at 37° C. (Landers et al., 2019a). In comparison, human fibrinogen (5 mg/mL) was buffer exchanged to pH 3.5 Tris buffer using AMICRON® ultra-4 centrifugal filter unit (UFC8010, Millipore Sigma, Burlington, MA), and then incubated with a mixture of secreted aspartic proteinases (Saps) containing predominantly Saps 1-3 isolated as described previously (Schild et al., 2011), or recombinant Saps (Schild et al., 2011), individually or combined at 0.02 mg/mL. Protein lysates were then prepared using NUPAGE™ 4-12% Bis-Tris Protein Gels, MES SDS Running Buffer, and SIMPLYBLUE™ SafeStain (Invitrogen, Carlsbad, CA). Samples for protein electrophoresis were diluted at least 1:2 in Tricine sample buffer and PRECISION PLUS PROTEIN™ Kaleidoscope Prestained Protein Standards were used as the protein electrophoresis molecular weight marker (Bio-Rad, Hercules, CA). See FIG. 12.

### F. A1A2A3 Von Willebrand Factor Tridomain Protein

Recombinant VWF A1A2A3 tridomain protein was generated using complementary DNA encoding the human VWF A1, A2, and A3 domains (amino acids Q1238-G1874) and inserted via PCR into the pSecTag2B vector (Invitrogen, CA) as described elsewhere (Auton 2007). Recombinant A1A2A3 was expressed in human embryonic kidney (HEK293) cells and purified using affinity chromatography from conditioned medium. The purified A1A2A3 protein was dialyzed against 1× tris-buffered saline supplemented with 0.1% tween-20 and subjected to gel electrophoresis for verification of purity prior to experimental use (Auton et al., 2007).

### G. Fungal Cultivation

Sabouraud's agar (BD, Sparks, 21152) was dissolved in water at 50 g/L, and autoclaved for 30 min. Chloramphenicol (Sigma Aldrich, St. Louis) was added to the warm solution at 50 mg/L and the agar was sterilely poured into Petri dishes (VWR, Radnor, 19087) and cooled overnight. Plates were sealed and kept at 4° C. until used for fungal growth.

### H. Induction and Quantitation of Allergic Airway Disease

C57BL/6 mice were administered 1×10⁵ viable cells of C. albicans intranasally every other day for 8 challenges as shown in FIG. 1 (Porter et al., 2011a). Alternatively, C57BL/6 mice were given 4, 8, and 16 μmol candidalysin or 16 μmol of scrambled peptide control in PBS every other day for 8 challenges as shown in FIG. 8. Dkk-1 inhibitor (Chae et al., 2016a) (WAY262611, cat: 317700, Millipore Sigma, Burlington, MA. Dose: 10 or 20 μg/kg) and recombinant mouse Dkk-1 (5897-DK-010, R&D systems, Minneapolis, MN. Dose: 5 μg/kg) were given intraperitoneally at the same time and fungal challenge. Mice were analyzed 24 h after the final challenge for allergic airway disease endpoints.

Bronchial constriction in response to increasing dose of acetylcholine (Ach) injected intravenously, bronchoalveolar lavage fluid (BALF) cell differential counting, Dkk-1 quantitation from plasma, ELISA analysis of whole lung, flow cytometry analysis of Th cells and lung histopathology were assessed in response to C. albicans challenge as previously described (Porter et al., 2011a). Briefly, mice were anesthetized with etomidate and intravenously injected with acetylcholine via tail vein to assess AHR as quantified by measuring respiratory system resistance (RRS). Total BALF cells were collected by lavaging whole lung, total cells were enumerated and differential cell counting was performed on modified Giemsa-stained cytospin preparations. Plasma was harvested by retro-orbital bleeding and anti-coagulated by 10% 0.5M EDTA (Thermofisher scientific, Waltham MA). Lungs were harvested and processed as follows. Lungs were cut into small pieces and incubated in digestion buffer (2 mg/ml collagenase (#LS004177, Worthington), 0.04 mg/ml DNAse (#10104159001, Sigma) 1, 20% FBS in HBSS) for 1 h at 37° C. after which they were deaggregated by pressing through a 40 μM nylon mesh and centrifuged at 400×g for 5 minutes at 4° C. Supernatants were discarded, and 1.5 mL of ACK (Thermofisher scientific, Waltham MA) was added and incubated for 3 min at room temperature for erythrocyte lysis. ACK was then neutralized with 7.5 mL of complete RPMI-1640 (Corning, NY), with 10% FBS and 1% Pen Strep, Gibco, Waltham MA). The resulting leukocyte preparations were centrifuged and prepared for flow cytometry analysis or ELISA.

### I. Cytokine Measurements

Cell culture supernatants were analyzed for cytokines by standard ELISA after comparison to recombinant standard. IL-4 (Clone 11B11 and 554390 BD Biosciences, San Jose, CA), IL-5, IL-13 (DY405, DY413, R&D systems, Minneapolis, MN), IL-17 (BMS2017, Thermofisher scientific, Waltham MA), IFN-7 (555142, BD Biosciences, San Jose, CA), IL-1, IL-6, TNF (ab210895, ab213749 and ab212073, Abcam, Cambridge MA) were used as antibodies pairs and capture signals were amplified using Streptavidin-horseradish peroxidase conjugate (51-9002813, BD Biosciences, San Jose, CA). The plate was further developed using TMB substrate solution (N301, Thermofisher scientific, Waltham MA) and detected at the absorbance wavelength of 450 nm.

### J. Flow Cytometry

Total lung cells isolated above were stained with Live/Dead Fix Blue (L34961, Thermofisher scientific, Waltham MA), CD45, CD3, CD4 (103122, 100222, 100412, Biolegend, San Diego, CA). Cells were separated into 3 groups, then permeabilized and fixed using Transcription Factor Buffer Set (562574, BD Biosciences, San Jose, CA), and stained individually for T-bet, GATA3 or RORγt (561265, 560074, 562607, BD Biosciences, San Jose, CA). For megakaryocyte staining, total lung cells were stained with Live/Dead Fix Blue and CD41, CXCR4 (133927, 146507, Biolegend, San Diego, CA), Dkk-1 (BAM1765, R&D systems, Minneapolis, MN) and PE-streptavidin (405203, Biolegend, San Diego, CA). Data were analyzed using FlowJo software (version 10.0.7; Treestar, Ashland, OR).

K. Measurement of Dkk-1 from Plasma

Whole blood from mice was isolated by retro-orbital puncture and anticoagulated with 10% 0.5 M EDTA and plasma was isolated by centrifugation at 1000×g for 10 min at 4° C. and stored at −80° C. until analyzed. Plasma from either mice or humans was diluted 1:10 for Dkk-1 measurement by ELISA (DY1765, R&D systems, Minneapolis, MN) according to the manufacturer's protocol. For human Dkk-1 quantitation, previously banked plasma samples obtained from subjects presenting for evaluation of either chronic obstructive pulmonary disease (COPD) and no prior history of asthma or CRS or CRSwNP were used. Specimens were randomly selected for analysis, excluding those with substantial hemolysis or that demonstrated platelet contamination.

L. Platelet Isolation from Mice

Whole blood from mice was isolated by retro-orbital bleeding and anticoagulated with 10% 0.5 M EDTA. Alternatively, whole blood was collected from the left or the right ventricle of mice using an 18G needle pre-coated with 10% 0.5 M EDTA. Platelet rich plasma was isolated by centrifugation at 180×g for 10 min at room temperature, and platelets were isolated by centrifugation at 1250×g for 10 min at room temperature. Platelets were then resuspended in Tyrode's buffer (NaCl: 8.19 g/L, KCl: 0.2 g/L, NaHCO₃: 1.01 g/L, NaH₂PO₄, 0.055 g/L, Glucose: 0.991 g/L, MgCl₂: 0.49 mM, CaCl₂): 1.8 mM) and counted via flow cytometry. For Dkk-1 quantification, platelets were resuspended in PBS and lysed via sonication.

M. Preparation of human platelets

Human platelets were isolated from whole blood as platelet rich plasma through the Gulf Coast Blood Center, Houston, TX. For washed, plasma free platelets, PGE1 (sc-201223, Santa Cruz, Dallas, TX) was added to platelet rich plasma to a working concentration of 75 nM. Platelet rich plasma was then centrifuged at 1000×g for 10 min. The supernatant, platelet poor plasma, was removed using a pipette and platelets were resuspended in CGS buffer (13 mM sodium citrate, 30 mM glucose, 120 mM NaCl, pH: 6.25) containing 75 nM PGE1. Platelets were then centrifuged at 1000×g for 10 min again. The supernatant was removed and the platelet pellet was resuspended in Tyrode's buffer.

N. Dkk-1 release assay

Human platelets (1×10⁹/mL) were pretreated with/without receptor blocker for 1 h at 37° C. (P2Y1: Clopidogrel (Weber et al., 1999), P2Y12: MRS2179 (Baurand et al., 2001), GP2b3a: tirofiban, GPVI: losartan (Taylor et al., 2014) at 100 μM, Millipore Sigma, Burlington, MA; α2β1: 10 μg/mL 910901, Biolegend, San Diego, CA; GPIV: 10 μg/mL ab23680, Abcam, Cambridge MA; GPVI: 10 μg/mL AF3627, CLEC2: 10 μg/mL AF1718 (Tsukiji et al., 2018), R&D systems, Minneapolis, MN; TLR4: 10 μg/mL tlrl-prslps, Invivogen, San Diego, CA; Anti-GP1bα Clone AK2 (Yuan et al., 1999) at 10 μg/mL, Invitrogen, Carlsbad, CA) and then incubated with candidalysin (10 or 20 μM) or Candida albicans overnight at 37° C. Cells were centrifuged at 1000×g, 10 min at 4° C. and the supernatants were again centrifuged at 20,000×g, 10 min, 4° C. to remove platelets and yeast cells. The remaining supernatants were diluted 1:10 for Dkk-1 measurement by ELISA (DY1906, R&D systems, Minneapolis, MN).

Alternatively, EOMA cells were seeded in 24 well plates (2×10⁶/well) and stimulated with candidalysin (10 or 20 μM) overnight at 37° C. Cells were centrifuged at 400×g, 5 min at 4° C. and the supernatants were used to detect Dkk-1 by ELISA (DY1765, R&D systems, Minneapolis, MN). The lower limit of detection of Dkk-1 in tissue culture media and PBS was 80-100 μg/ml and in plasma, 1.5-2.2 ng/ml.

O. Platelet activation and binding to candidalysin

Platelets (1×10⁹/mL) were pretreated with anti-GP1bα (Clone AK2 at 10 μg/mL, Invitrogen, Carlsbad, CA) or A1A2A3 von Willebrand factor tridomain protein for 1 h at room temperature and then incubated with untagged, Alexafluor-647-tagged candidalysin (10 or 20 μM), or scrambled peptide control for 1 h at room temperature. Activation was assessed by CD62P upregulation (304910, Biolegend, San Diego, CA) and AF-647-candidalysin binding to platelets was assessed by flow cytometry (Nagy Jr et al., 2013).

Alternatively, platelets were treated with biotinylated candidalysin (10 or 20 μM) or scrambled peptide control (20 μM) for 1 hour, washed with CGS buffer, and stained using ALEXA FLUOR® 647-tagged streptavidin for 30 minutes.

### P. GP1bα-Candidalysin Binding Assay

96-well plates (9018, Corning, Kennebuck, ME) were coated with 5 μg/mL candidalysin, scrambled peptide control in carbonate buffer (pH=9.0) overnight at 4° C. Plates were blocked with I-BLOCK™ (2%) for 2 h at 37° C. and incubated with a 2/3 serial dilution of His-tagged GP1bα (4067-GP, R&D systems, Minneapolis, MN) starting from 50 nM for 2 h at 37° C. After washing, plates were incubated with biotinylated anti-His antibody (5 μg/mL, BAM050, R&D systems, Minneapolis, MN) for 2 h at 37° C. followed by SAv-HRP (1:250, 51-9002813, BD Biosciences, San Jose, CA). The plate was further developed using TMB substrate solution (N301, Thermofisher scientific, Waltham MA) and detected at the absorbance wavelength of 450 nm. Alternatively, plates were coated with 5 μg/mL GP1bα and a 2/3 serial dilution of biotinylated candidalysin or scrambled peptide control starting from 5 nM was performed followed by addition of SAv-HRP and TMB. As a proper control, binding affinity between candidalysin and an integrin receptor GPIIb/IIIa was also assessed in the same experimental setup (FIG. 10).

### Q. Inhibition of GP1bα-Candidalysin Binding

96 well plates (9018, Corning, Kennebuck, ME) were coated with 0.1 μg/mL GP1bα in carbonate buffer (pH=9.0) overnight at 4° C. Plates were blocked for 2 h at 37° C. and incubated with a 1/2 serial dilution of anti-GP1bα antibody (Clone AK2, Invitrogen, Carlsbad, CA) or VWF A1A2A3 tridomain protein for 2 h at 37° C. Without washing, biotinylated candidalysin was added into each well to a final concentration of 0.1 μM and incubated for 1 h at 37° C. followed by SAv-HRP and TMB.

### R. GP1bα Pulldown

Human platelets were lysed using ChIP lysis buffer (5 mM PIPES, 85 mM KCl, 0.5% NP-40. P7643, P3911, 98379, Sigma Aldrich, St. Louis) and lysates were then buffer transferred to TBS using AMICON® filter unit (UFC801096, Sigma Aldrich, St. Louis) containing protease inhibitor cocktail (78442, Thermofisher scientific, Waltham MA). Immunoprecipitation was carried out using Pierce biotinylated protein interaction pulldown kit (21115 Thermofisher Scientific, Waltham MA). Briefly, 60 μg of biotinylated candidalysin or scrambled peptide control were loaded onto agarose-streptavidin slurry as bait proteins. After biotin blocking, the slurry was incubated with platelet lysate for 1 h at 4° C. The slurry was then washed with acetate buffer containing 0.5 M NaCl, and the eluates were subjected to SDS-PAGE using 5% milk as blocking reagent to detect GP1bα (2 μg/mL, MAB4067, R&D systems, Minneapolis, MN).

### S. Platelet Aggregation Assay

Blood from healthy individuals was drawn and mixed with sodium citrate. Blood was centrifuged at 189×g for 15 min at room temperature to obtain platelet-rich plasma (PRP). Platelet aggregation was initiated by addition of collagen (2.5 μg/mL) or increasing concentrations of candidalysin to a 225 μl aliquot of PRP in a four channel Bio/Data PAP-4C aggregometer (Biodata Corporation, Horsham, PA). Platelet aggregation was then assessed after 5 minutes.

### T. Platelet Depletion

Wildtype mice were give 2 μg/g of platelet depleting antibody intraperitoneally (R300, Emfret Analytics, Wurzburg, Germany) as described (Lam et al., 2011). Depletion of platelets from mice in the allergic airway disease model was carried out with intraperitoneal injection of the antibody with each intranasal challenge, 8 times over 2 weeks as described above.

### U. Hemoglobin Quantitation

Bronchoalveolar lavage fluid from mice was obtained as above. Free hemoglobin was quantified using a colorimetric kit (ab234046, Abcam, Cambridge MA).

### V. Lung Section Staining

Lung sections were isolated from mice and stained using H&E, PAS kit or GMS kit (1016460001, 1008200007, Sigma Aldrich, St. Louis).

### W. Measurement of Lung Density and Volumes

Mice were anesthetized, intubated and placed into the CT scanner to generate images. VIVOQUANT™ image analysis solution software (Invicro, Boston, MA) was used for post-processing of DICOM image data. To determine lung density in Hounsfield unit (HU), the 3D ROI tool was used to generate quantitative analysis. An internal negative and positive control were measured based on the phantom pane located on each image. A standard anatomical landmark for HU measurement was determined for each lung sample. Using the coronal plane, the right and left bronchi were identified, and a 10-point mark was placed lateral to each bronchi. The mean HU per lung was calculated and compared to the negative control HU and positive control HU. Quantitation of lung volume was calculated using segmentation algorithms. OTSU thresholding and ROI connected thresholding functional tools were utilized by measuring the number of voxels assigned to the lung space. Lung volumes, measured in mm³, represent clear lung space within the lungs.

### X. Quantification and Statistical Analysis

Data are presented as means±standard errors of the means. Significant differences relative to PBS-challenged mice or appropriate controls are expressed by P values of <0.05, as measured two tailed Student's t-test or one-way ANOVA followed by Dunnett's test or Tukey's test for multiple comparison. Survival curves were analyzed using Log-rank test. Human plasma samples were analyzed using Kruskal-Wallis test. Data normality was confirmed using the Shapiro-Wilk test.

All of the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Auton, M., Cruz, M. A., and Moake, J. (2007). Conformational stability and domain unfolding of the Von Willebrand factor A domains. Journal of molecular biology 366, 986-1000.
Azuma, H., Dent, J. A., Sugimoto, M., Ruggeri, Z. M., and Ware, J. (1991). Independent assembly and secretion of a dimeric adhesive domain of von Willebrand factor containing the glycoprotein Ib-binding site. J Biol Chem 266, 12342-12347.
Baum, G. L. (1960). The significance of Candida albicans in human sputum. N Engl J Med 263, 70-73.
Baurand, A., Raboisson, P., Freund, M., Ldon, C., Cazenave, J.-P., Bourguignon, J.-J., and Gachet, C. (2001). Inhibition of platelet function by administration of MRS2179, a P2Y1 receptor antagonist. European journal of pharmacology 412, 213-221.
Beckert, H., Meyer-Martin, H., Buhl, R., Taube, C., and Reuter, S. (2018). The Canonical but Not the Noncanonical Wnt Pathway Inhibits the Development of Allergic Airway Disease. Journal of Immunology 201, 1855-1864.
Boos, A. C., Hagl, B., Schlesinger, A., Halm, B. E., Ballenberger, N., Pinarci, M., Heinz, V., Kreilinger, D., Spielberger, B. D., Schimke-Marques, L. F., et al. (2014). Atopic dermatitis, STAT3- and DOCK8-hyper-IgE syndromes differ in IgE-based sensitization pattern. Allergy 69, 943-953.
Calvete, J. J. (1995). On the structure and function of platelet integrin alIbP3, the fibrinogen receptor. Proceedings of the Society for Experimental Biology and Medicine 208, 346-360.
Chae, W.-J., Ehrlich, A. K., Chan, P. Y., Teixeira, A. M., Henegariu, O., Hao, L., Shin, J. H., Park, J.-H., Tang, W. H., and Kim, S.-T. (2016a). The Wnt antagonist Dickkopf-1 promotes pathological type 2 cell-mediated inflammation. Immunity 44, 246-258.
Chae, W. J., Ehrlich, A. K., Chan, P. Y., Teixeira, A. M., Henegariu, O., Hao, L., Shin, J. H., Park, J. H., Tang, W. H., Kim, S. T., et al. (2016b). The Wnt Antagonist Dickkopf-1 Promotes Pathological Type 2 Cell-Mediated Inflammation. Immunity 44, 246-258.
Chu, E. Y., Freeman, A. F., Jing, H., Cowen, E. W., Davis, J., Su, H. C., Holland, S. M., and Turner, M. L. (2012). Cutaneous manifestations of DOCK8 deficiency syndrome. Archives of Dermatology 148, 79-84.
Dinkla, S., van Cranenbroek, B., van der Heijden, W. A., He, X., Wallbrecher, R., Dumitriu, LE., van der Ven, A. J., Bosman, G. J., Koenen, H. J., and Joosten, I. (2016). Platelet microparticles inhibit IL-17 production by regulatory T cells through P-selectin. Blood 127, 1976-1986.
Engelhardt, K. R., Gertz, M. E., Keles, S., Schaffer, A. A., Sigmund, E. C., Glocker, C., Saghafi, S., Pourpak, Z., Ceja, R., Sassi, A., et al. (2015). The extended clinical phenotype of 64 patients with dedicator of cytokinesis 8 deficiency. Journal of Allergy & Clinical Immunology 136, 402-412.
Eppinger, T. M., Greenberger, P. A., White, D. A., Brown, A. E., and Cunningham-Rundles, C. (1999). Sensitization to Aspergillus species in the congenital neutrophil disorders chronic granulomatous disease and hyper-IgE syndrome. J. Allergy Clin. Immunol. 104, 1265-1272.
Fahy, J. V. (2001). Remodeling of the airway epithelium in asthma. Am J Respir Crit Care Med 164, S46-51.
Gropp, K., Schild, L., Schindler, S., Hube, B., Zipfel, P. F., and Skerka, C. (2009). The yeast Candida albicans evades human complement attack by secretion of aspartic proteases. Molecular Immunology 47, 465-475.
Harb, H., Stephen-Victor, E., Crestani, E., Benamar, M., Massoud, A., Cui, Y., Charbonnier, L. M., Arbag, S., Baris, S., Cunnigham, A., et al. (2020). A regulatory T cell Notch4-GDF15 axis licenses tissue inflammation in asthma. 1, 1359-1370.
Ho, J., Wickramasinghe, D. N., Nikou, S. A., Hube, B., Richardson, J. P., and Naglik, J. R. (2020). Candidalysin Is a Potent Trigger of Alarmin and Antimicrobial Peptide Release in Epithelial Cells. Cells 9.
Huang, E., and Church, J. A. (2018). Chronic rhinosinusitis with severe nasal polyposis in hyper-IgE syndrome. Annals of Allergy, Asthma, & Immunology 121, 738-739.
Huang, H., and Cantor, A. B. (2009). Common features of megakaryocytes and hematopoietic stem cells: what's the connection? Journal of cellular biochemistry 107, 857-864.
Hube, B., Sanglard, D., Odds, F. C., Hess, D., Monod, M., Schafer, W., Brown, A. J., and Gow, N. A. (1997). Disruption of each of the secreted aspartyl proteinase genes SAP1, SAP2, and SAP3 of Candida albicans attenuates virulence. Infection & Immunity 65, 3529-3538.
Isenberg, W. M., McEver, R. P., Shuman, M. A., and Bainton, D. F. (1986). Topographic distribution of a granule membrane protein (GMP-140) that is expressed on the platelet surface after activation: an immunogold-surface replica study. Blood Cells 12, 191-204.
Ivanov, II, McKenzie, B. S., Zhou, L., Tadokoro, C. E., Lepelley, A., Lafaille, J. J., Cua, D. J., and Littman, D. R. (2006). The orphan nuclear receptor RORgammat directs the differentiation program of proinflammatory IL-17+T helper cells. Cell 126, 1121-1133.
Jackson, B. E., Wilhelmus, K. R., and Hube, B. (2007). The role of secreted aspartyl proteinases in Candida albicans keratitis. Investigative Ophthalmology & Visual Science 48, 3559-3565.
Kammer, P., McNamara, S., Wolf, T., Conrad, T., Allert, S., Gerwien, F., Hunniger, K., Kurzai, O., Guthke, R., Hube, B., et al. (2020). Survival Strategies of Pathogenic Candida Species in Human Blood Show Independent and Specific Adaptations. mBio 11, 06.
Kanaji, T., Russell, S., Cunningham, J., Izuhara, K., Fox, J. E., and Ware, J. (2004). Megakaryocyte proliferation and ploidy regulated by the cytoplasmic tail of glycoprotein Ibalpha. 1, 3161-3168.
Kasper, L., Konig, A., Koenig, P. A., Gresnigt, M. S., Westman, J., Drummond, R. A., Lionakis, M. S., Gros, O., Ruland, J., Naglik, J. R., and Hube, B. (2018). The fungal peptide toxin Candidalysin activates the NLRP3 inflammasome and causes cytolysis in mononuclear phagocytes. Nature communications 9, 4260.
Kheradmand, F., Kiss, A., Xu, J., Lee, S. H., Kolattukudy, P. E., and Corry, D. B. (2002). A protease-activated pathway underlying Th cell type 2 activation and allergic lung disease. J. Immunol. 169, 5904-5911.
Knutsen, A. P., Bush, R. K., Demain, J. G., Denning, D. W., Dixit, A., Fairs, A., Greenberger, P. A., Kariuki, B., Kita, H., Kurup, V. P., et al. (2012). Fungi and allergic lower respiratory tract diseases. Journal of Allergy & Clinical Immunology 129, 280-291.
Kwak, H. J., Park, D. W., Seo, J. Y., Moon, J. Y., Kim, T. H., Sohn, J. W., Shin, D. H., Yoon, H. J., Park, S. S., and Kim, S. H. (2015). The Wnt/beta-catenin signaling pathway regulates the development of airway remodeling in patients with asthma. Exp Mol Med 47, e198.
Lam, F. W., Burns, A. R., Smith, C. W., and Rumbaut, R. E. (2011). Platelets enhance neutrophil transendothelial migration via P-selectin glycoprotein ligand-1. American Journal of Physiology-Heart and Circulatory Physiology 300, H468-H475.
Landers, C. T., Tung, H.-Y., Knight, J. M., Madison, M. C., Wu, Y., Zeng, Z., Porter, P. C., Rodriguez, A., Flick, M. J., and Kheradmand, F. (2019a). Selective cleavage of fibrinogen by diverse proteinases initiates innate allergic and antifungal immunity through CD11b. Journal of Biological Chemistry 294, 8834-8847.
Landers, C. T., Tung, H. Y., Knight, J. M., Madison, M. C., Wu, Y., Zeng, Z., Porter, P. C., Rodriguez, A., Flick, M. J., Kheradmand, F., and Corry, D. B. (2019b). Selective cleavage of fibrinogen by diverse proteinases initiates innate allergic and antifungal immunity through CD11b. Journal of Biological Chemistry 294, 8834-8847.
Lefrancais, E., Ortiz-Munoz, G., Caudrillier, A., Mallavia, B., Liu, F., Sayah, D. M., Thornton, E. E., Headley, M. B., David, T., Coughlin, S. R., et al. (2017). The lung is a site of platelet biogenesis and a reservoir for haematopoietic progenitors. Nature 544, 105-109.
Li, E., Knight, J. M., Wu, Y., Luong, A., Rodriguez, A., Kheradmand, F., and Corry, D. B. (2019). Airway mycosis in allergic airway disease. Adv Immunol 142, 85-140.
Li, E., Tsai, C.-L., Maskatia, Z. K., Kakkar, E., Porter, P., Rossen, R. D., Perusich, S., Knight, J. M., Kheradmand, F., and Corry, D. B. (2018). Benefits of Antifungal Therapy in Asthma Patients with Airway Mycosis: A Retrospective Cohort Analysis. Immun. Inflamm. Dis. 6, 264-275.
Ma, C. S., Chew, G. Y., Simpson, N., Priyadarshi, A., Wong, M., Grimbacher, B., Fulcher, D. A., Tangye, S. G., and Cook, M. C. (2008). Deficiency of Th17 cells in hyper IgE syndrome due to mutations in STAT3. J Exp Med 205, 1551-1557.
Mak, G., Porter, P. C., Bandi, V., Kheradmand, F., and Corry, D. B. (2013). Tracheobronchial mycosis in a retrospective case-series study of five status asthmaticus patients. Clinical Immunology 146, 77-83.
Masaki, K., Fukunaga, K., Matsusaka, M., Kabata, H., Tanosaki, T., Mochimaru, T., Kamatani, T., Ohtsuka, K., Baba, R., Ueda, S., et al. (2017). Characteristics of severe asthma with fungal sensitization. Annals of Allergy, Asthma, & Immunology 119, 253-257.
Meiller, T. F., Hube, B., Schild, L., Shirtliff, M. E., Scheper, M. A., Winkler, R., Ton, A., and Jabra-Rizk, M. A. (2009). A novel immune evasion strategy of Candida albicans: proteolytic cleavage of a salivary antimicrobial peptide. PLoS ONE 4, e5039.
Meinders, M., Hoogenboezem, M., Scheenstra, M. R., De Cuyper, I. M., Papadopoulos, P., Nemeth, T., Mocsai, A., van den Berg, T. K., Kuijpers, T. W., and Gutierrez, L. (2016). Repercussion of Megakaryocyte-Specific Gatal Loss on Megakaryopoiesis and the Hematopoietic Precursor Compartment. PLoS ONE 1, e0154342.
Millien, V. O., Lu, W., Mak, G., Yuan, X., Knight, J. M., Porter, P., Kheradmand, F., and Corry, D. B. (2014). Airway Fibrinogenolysis and the initiation of allergic inflammation. AnnalsATS 11 (suppl 5), S277-S283.
Millien, V. O., Lu, W., Shaw, J., Yuan, X., Mak, G., Roberts, L., Song, L. Z., Knight, J. M., Creighton, C. J., Luong, A., et al. (2013). Cleavage of fibrinogen by proteinases elicits allergic responses through Toll-like receptor 4. Science 341, 792-796.
Milner, J. D., Brenchley, J. M., Laurence, A., Freeman, A. F., Hill, B. J., Elias, K. M., Kanno, Y., Spalding, C., Elloumi, H. Z., Paulson, M. L., et al. (2008). Impaired T(H)17 cell differentiation in subjects with autosomal dominant hyper-IgE syndrome. Nature 452, 773-776.
Moyes, D. L., Wilson, D., Richardson, J. P., Mogavero, S., Tang, S. X., Wernecke, J., Hofs, S., Gratacap, R. L., Robbins, J., Runglall, M., et al. (2016). Candidalysin is a fungal peptide toxin critical for mucosal infection. Nature 532, 64-68.
Naglik, J. R., Challacombe, S. J., and Hube, B. (2003). Candida albicans secreted aspartyl proteinases in virulence and pathogenesis. Microbiol Mol Biol Rev 67, 400-428, table of contents.
Nagy Jr, B., Debreceni, I. B., and Kappelmayer, J. (2013). Flow cytometric investigation of classical and alternative platelet activation markers. EJIFCC 23, 124.
Page, C., and Pitchford, S. (2014). Platelets and allergic inflammation. Clinical & Experimental Allergy 44, 901-913.
Pene, J., Chevalier, S., Preisser, L., Venereau, E., Guilleux, M. H., Ghannam, S., Moles, J. P., Danger, Y., Ravon, E., Lesaux, S., et al. (2008). Chronically inflamed human tissues are infiltrated by highly differentiated Th17 lymphocytes. J. Immunol. 180, 7423-7430.
Pitchford, S. C., Momi, S., Baglioni, S., Casali, L., Giannini, S., Rossi, R., Page, C. P., and Gresele, P. (2008). Allergen induces the migration of platelets to lung tissue in allergic asthma. American Journal of Respiratory & Critical Care Medicine 177, 604-612.
Pitchford, S. C., Riffo-Vasquez, Y., Sousa, A., Momi, S., Gresele, P., Spina, D., and Page, C. P. (2004). Platelets are necessary for airway wall remodeling in a murine model of chronic allergic inflammation. Blood 103, 639-647.
Porter, P., A., F., Qian, Y., Abramson, S., Delclos, G. L., Kheradmand, F., and Corry, D. B. (2011a). Necessary and sufficient role for T helper cells to prevent fungal dissemination during mucosal airway infection. Infect. Immun. 79, 4459-4471.
Porter, P., Polikepahad, S., Qian, Y., Knight, J. M., Lu, W., Tai, W. M., Roberts, L., Ongeri, V., Yang, T., Seryshev, A., et al. (2011b). Respiratory tract allergic disease and atopy: experimental evidence for a fungal infectious etiology. Med. Mycol. 49, S158-163.
Porter, P., Susarla, S. C., Polikepahad, S., Qian, Y., Hampton, J., Kiss, A., Vaidya, S., Sur, S., Ongeri, V., Yang, T., et al. (2009). Link between allergic asthma and airway mucosal infection suggested by proteinase-secreting household fungi. Mucosal Immunol. 2, 504-517.
Porter, P. C., Lim, D. J., Maskatia, Z. K., Mak, G., Tsai, C. L., Citardi, M. J., Fakhri, S., Shaw, J. L., Fothergil, A., Kheradmand, F., et al. (2014). Airway surface mycosis in chronic T_{H}2-associated airway disease. J Allergy Clin Immunol 134, 325-331.
Porter, P. C., Ongeri, V., Luong, A., Kheradmand, F., and Corry, D. B. (2011c). Seeking common pathophysiology in asthma, atopy and sinusitis. Trends Immunol. 32, 43-49.
Rayes, J., Bourne, J. H., Brill, A., and Watson, S. P. (2020). The dual role of platelet-innate immune cell interactions in thrombo-inflammation. Res Pract Thromb Haemost 4, 23-35.
Sambrano, G. R., Weiss, E. J., Zheng, Y. W., Huang, W., and Coughlin, S. R. (2001). Role of thrombin signalling in platelets in haemostasis and thrombosis. Nature. 413, 74-78.
Sandhu, R. S., Mehta, S. K., Khan, Z. U., and Singh, M. M. (1979). Role of Aspergillus and Candida species in allergic bronchopulmonary mycoses. A comparative study. Scandinavian Journal of Respiratory Diseases 60, 235-242.
Sanglard, D., Hube, B., Monod, M., Odds, F. C., and Gow, N. A. (1997). A triple deletion of the secreted aspartyl proteinase genes SAP4, SAP5, and SAP6 of Candida albicans causes attenuated virulence. Infection & Immunity 65, 3539-3546.
Schaller, M., Korting, H. C., Schafer, W., Bastert, J., Chen, W., and Hube, B. (1999). Secreted aspartic proteinase (Sap) activity contributes to tissue damage in a model of human oral candidosis. Molecular Microbiology 34, 169-180.
Schaller, M., Schackert, C., Korting, H. C., Januschke, E., and Hube, B. (2000). Invasion of Candida albicans correlates with expression of secreted aspartic proteinases during experimental infection of human epidermis. Journal of Investigative Dermatology 114, 712-717.
Schild, L., Heyken, A., de Groot, P. W., Hiller, E., Mock, M., de Koster, C., Horn, U., Rupp, S., and Hube, B. (2011). Proteolytic cleavage of covalently linked cell wall proteins by Candida albicans Sap9 and Sap10. Eukaryotic cell 10, 98-109.
Shah, S. A., Page, C. P., and Pitchford, S. C. (2017). Platelet-Eosinophil Interactions As a Potential Therapeutic Target in Allergic Inflammation and Asthma. Frontiers in Medicine 4, 129.
Shattil, S. J. (1999). Signaling through platelet integrin αIIbβ3: inside-out, outside-in, and sideways. Thrombosis and haemostasis 82, 318-325.
Shi, G., Field, D. J., Ko, K. A., Ture, S., Srivastava, K., Levy, S., Kowalska, M. A., Poncz, M., Fowell, D. J., and Morrell, C. N. (2014). Platelet factor 4 limits Th17 differentiation and cardiac allograft rejection. Journal of Clinical Investigation 124, 543-552.
Stoiber, D., and Assinger, A. (2020). Platelet-Leukocyte Interplay in Cancer Development and Progression. Cells 9, 01.
Suh, D. H., Trinh, H. K., Liu, J. N., Pham le, D., Park, S. M., Park, H. S., and Shin, Y. S. (2016). P2Y12 antagonist attenuates eosinophilic inflammation and airway hyperresponsiveness in a mouse model of asthma. Journal of Cellular & Molecular Medicine 20, 333-341.
Sullivan, P. J., Jafar, Z. H., Harbinson, P. L., Restrick, L. J., Costello, J. F., and Page, C. P. (2000). Platelet dynamics following allergen challenge in allergic asthmatics. Respiration 67, 514-517.
Svenningsen, S., and Nair, P. (2017). Asthma Endotypes and an Overview of Targeted Therapy for Asthma. Front Med (Lausanne) 4, 158.
Szabo, S. J., Kim, S. T., Costa, G. L., Zhang, X., Fathman, C. G., and Glimcher, L. H. (2000). A novel transcription factor, T-bet, directs Th1 lineage commitment. Cell 100, 655-669.
Taylor, L., Vasudevan, S. R., Jones, C. I., Gibbins, J. M., Churchill, G. C., Campbell, R. D., and Coxon, C. H. (2014). Discovery of novel GPVI receptor antagonists by structure-based repurposing. PloS one 9.
Tischler, B. Y., Tosini, N. L., Cramer, R. A., and Hohl, T. M. (2020). Platelets are critical for survival and tissue integrity during murine pulmonary Aspergillus fumigatus infection. PLoS Pathog 16.
Tsukiji, N., Osada, M., Sasaki, T., Shirai, T., Satoh, K., Inoue, O., Umetani, N., Mochizuki, C., Saito, T., and Kojima, S. (2018). Cobalt hematoporphyrin inhibits CLEC-2-podoplanin interaction, tumor metastasis, and arterial/venous thrombosis in mice. Blood advances 2, 2214-2225.
Verma, A. H., Richardson, J. P., Zhou, C., Coleman, B. M., Moyes, D. L., Ho, J., Huppler, A. R., Ramani, K., McGeachy, M. J., Mufazalov, I. A., et al. (2017). Oral epithelial cells orchestrate innate type 17 responses to Candida albicans through the virulence factor candidalysin. Sci Immunol 2, 03.
Verma, A. H., Zafar, H., Ponde, N. O., Hepworth, O. W., Sihra, D., Aggor, F. E. Y., Ainscough, J. S., Ho, J., Richardson, J. P., Coleman, B. M., et al. (2018). IL-36 and IL-1/IL-17 Drive Immunity to Oral Candidiasis via Parallel Mechanisms. J Immunol 201, 627-634.
Weber, A. A., Reimann, S., and Schrör, K. (1999). Specific inhibition of ADP—induced platelet aggregation by clopidogrel in vitro. British journal of pharmacology 126, 415-420.
Wu, Y., Du, S., Johnson, J. L., Tung, H.-Y., Landers, C. T., Liu, Y., Seman, B. G., Wheeler, R. T., Costa-Mattioli, M., Kheradmand, F., et al. (2019). Microglia and amyloid precursor protein coordinate control of transient Candida cerebritis with memory deficits. Nat. Commun. 10, 58.
Yuan, Y., Kulkarni, S., Ulsemer, P., Cranmer, S. L., Yap, C. L., Nesbitt, W. S., Harper, I., Mistry, N., Dopheide, S. M., and Hughan, S. C. (1999). The von Willebrand factor-glycoprotein Ib/V/IX interaction induces actin polymerization and cytoskeletal reorganization in rolling platelets and glycoprotein Ib/V/IX-transfected cells. Journal of Biological Chemistry 274, 36241-36251.
Zhao, Y., Yang, J., Gao, Y. D., and Guo, W. (2010). Th17 immunity inpatients with allergic asthma. International Archives of Allergy & Immunology 151, 297-307.
Zheng, W., and Flavell, R. A. (1997). The transcription factor GATA-3 is necessary and sufficient for Th2 cytokine gene expression in CD4 T cells. Cell 89, 587-596.

What is claimed is:

1. A method of treating or preventing an allergic airway disease in a subject in need thereof comprising administering to the subject an effective amount of a composition comprising one or more Dickkopf-1 (Dkk-1) inhibitors.

2. The method of claim 1, wherein the allergic airway disease comprises chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof.

3. The method of claim 1 or claim 2, wherein the allergic airway disease comprises asthma.

4. The method of any one of claims 1-3, wherein the allergic airway disease is caused at least in part by one or more irritants and/or immune activators comprising fungal infection, mold or mildew, hair or dander, dust, pollen, smoke, exercise, stress, perfume or other strong odors, air pollution comprising ozone, particulates, nitrogen dioxides, or sulfate aerosols, and/or changes in the weather.

5. The method of any one of claims 1-4, wherein the allergic airway disease is caused at least in part by fungal infection.

6. The method of claim 4 or claim 5, wherein the fungal infection comprises Aspergillus spp., Penicillium spp., Alternaria spp., Penicillium spp., Curvularia spp., Bipolaris, Mucor spp., Rhizopus spp., Pneumocystis spp., Aureobasidia spp., Cladosporium spp., Cochliobus spp., Paecilomyces spp., Trichoderma spp., Trichosporon spp., Malassezia spp., and/or Candida spp. fungi.

7. The method of any one of claims 4-6, wherein the fungal infection comprises Aspergillus spp.

8. The method of claim 7, wherein the Aspergillus fungi comprise Aspergillus flavus, Aspergillus fumigatus, Aspergillus nidulans, Aspergillus oryzae, Aspergillus sydowii, Aspergillus versicolor, Aspergillus wentii, and/or Aspergillus niger.

9. The method of any one of claims 4-8, wherein the fungal infection comprises Candida spp.

10. The method of claim 9, wherein the Candida fungi comprise Candida albicans, Candida tropicalis, Candida glabrata, Candida auris, Candida lusitaniae, Candida parapsilosis, Candida krusei, Candida dubliniensis, and/or Candida guilliermondii.

11. The method of any one of claims 1-10, wherein the Dkk-1 inhibitors inhibit Dkk-1 activity.

12. The method of any one of claims 1-11, wherein the Dkk-1 inhibitors inhibit release of Dkk-1 from platelets.

13. The method of any one of claims 1-12, wherein the one or more Dkk-1 inhibitors comprise a small molecule, an antibody, or a nucleic acid.

14. The method of any one of claims 1-13, wherein the one or more Dkk-1 inhibitors comprise WAY 262611, gallocyanine, NCI8642, or functional derivatives thereof.

15. The method of any one of claims 1-14, wherein the one or more Dkk-1 inhibitors comprise WAY 262611 or a functional derivative thereof.

16. The method of any one of claims 1-15, wherein the one or more Dkk-1 inhibitors comprise DKN-01 or a functional derivative thereof.

17. The method of any one of claims 1-16, wherein the allergic airway disease is resistant to treatment with a corticosteroid.

18. The method of any one of claims 1-17, wherein the allergic airway disease is characterized by one or more symptoms comprising inflammation, wheezing, coughing, chest pain or tightness, reversible airway obstruction, airway hyper-responsiveness, shortness of breath or difficulty breathing, excess mucus or watery secretions in the bronchial tubes, nasal passages, or sinuses, swollen mucous membrane in the bronchial tubes, nasal passages, or sinuses, hypersensitive bronchial tubes, nasal passages, or sinuses, headache, nasal congestion, loss of sense of smell, or a combination thereof.

19. The method of any one of claims 1-18, wherein treating or preventing the allergic airway disease comprises reducing airway inflammation.

20. The method of any one of claims 1-19, wherein treating or preventing the allergic airway disease comprises reducing airway hyper-responsiveness.

21. The method of any one of claims 1-20, wherein treating or preventing the allergic airway disease comprises inhibiting an adaptive or innate immune response by the subject.

22. The method of claim 21, wherein inhibiting the adaptive or innate immune response by the subject comprises inhibiting cytokine secretion and/or inhibiting recruitment or activity of inflammatory cells and/or T helper effector cells.

23. The method of claim 22, wherein the cytokines comprise interleukin-2, interleukin-4, interleukin-5, interleukin-6, interleukin-9, interleukin-10, interleukin-12, interleukin-13, interleukin-17A, interleukin-17B, interleukin-17C, interleukin-17D, interleukin-17E, interleukin-17F, interleukin-22, interleukin-33, tumor necrosis factor, thymic stromal lymphopoietin, ciliary neurotrophic factor, or interleukin-1β.

24. The method of claim 22 or claim 23, wherein the inflammatory cells comprise granulocytes and/or macrophages.

25. The method of claim 22, wherein the T helper effector cells comprise T helper type 2 (Th2) cells and/or T helper type 17 (Th17) cells.

26. The method of any one of claims 1-25, wherein the composition further comprises one or more pharmaceutically acceptable excipients.

27. The method of any one of claims 1-26, wherein the composition is administered intranasally, subcutaneously, intravenously, by aerosol, and/or orally.

28. The method of any one of claims 1-27, wherein the subject is provided an effective amount one or more additional therapies for the allergic airway disease.

29. The method of claim 28, wherein the one or more additional therapies comprise corticosteroids, leukotriene modifiers, bronchodilators, antifungals, biologics, allergy shots, antihistamines, decongestants, cromolyn, or combinations or derivatives thereof.

30. The method of claim 29, wherein the corticosteroids comprise fluticasone, dexamethasone, budesonide, mometasone, beclomethasone, ciclesonide, or combinations or derivatives thereof.

31. The method of claim 29, wherein the leukotriene modifiers comprise montelukast, zafirlukast, zileuton, or combinations or derivatives thereof.

32. The method of claim 29, wherein the bronchodilators comprise theophylline, albuterol, levalbuterol, ipratropium, aclidinium, arformoterol, formoterol, indacaterol, tiotropium, salmeterol, glycopyrrolate, olodaterol, vilanterol, umeclidinium, or combinations or derivatives thereof.

33. The method of claim 29, wherein the antifungals comprise amphotericin B, terbinafine, voriconazole, itraconazole, fluconazole, isavuconazole, posaconazole, ketoconazole, micafungin, ibrexafungerp, caspofungin, or combinations or derivatives thereof.

34. The method of claim 29, wherein the biologics comprise omalizumab, mepolizumab, benralizumab, dupilumab, reslizumab, or combinations or derivatives thereof.

35. The method of claim 29, wherein the antihistamines comprise azelastine, brompheniramine, cetirizine, chlorpheniramine, desloratadine, diphenhydramine, doxylamine, fexofenadine, hydroxyzine, ketotifen, loratadine, levocetirizine, olaptadine, or combinations or derivatives thereof.

36. The method of claim 29, wherein the decongestants comprise levmetamfetamine, naphazoline, pseudoephedrine, phenylephrine, propylhexedrine, oxymetazoline, xylometazoline, or combinations or derivatives thereof.

37. The method of any one of claims 28-36, wherein the one or more Dkk-1 inhibitors and one or more additional therapies are administered in the same composition.

38. The method of any one of claims 28-36, wherein the one or more Dkk-1 inhibitors and one or more additional therapies are administered in different compositions.

39. The method of any one of claims 1-38, wherein the composition is administered once or multiple times.

40. The method of claim 39, wherein when the composition is administered to the individual multiple times, the duration between administrations is within 1-24 hours, 1-7 days, 1-4 weeks, or 1-12 months.

41. The method of any one of claims 1-38, wherein when providing the composition to the subject, the subject had or was at risk of having chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof.

42. The method of any one of claims 1-38, further comprising the step of identifying that the subject had or was at risk of having chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof.

43. A method of reducing airway inflammation, reducing airway hyper-responsiveness, and/or inhibiting an adaptive or innate immune response in the airway of a subject comprising administering to the subject an effective amount of a composition comprising one or more Dickkopf-1 (Dkk-1) inhibitors.

44. The method of 38, wherein the inflammation, hyper-responsiveness, and/or adaptive or innate immune response is caused at least in part by one or more irritants and/or immune activators comprising fungal infection, mold or mildew, hair or dander, dust, pollen, smoke, exercise, stress, perfume or other strong odors, air pollution comprising ozone, particulates, nitrogen dioxides, or sulfate aerosols, and/or changes in the weather.

45. The method of claim 43 or claim 44, wherein the inflammation, hyper-responsiveness, and/or adaptive or innate immune response is caused at least in part by fungal infection.

46. The method of claim 44 or claim 45, wherein the fungal infection comprises Aspergillus spp., Penicillium spp., Alternaria spp., Penicillium spp., Curvularia spp., Bipolaris, Mucor spp., Rhizopus spp., Pneumocystis spp., Aureobasidia spp., Cladosporium spp., Cochliobus spp., Paecilomyces spp., Trichoderma spp., Trichosporon spp., Malassezia spp., and/or Candida spp. fungi.

47. The method of any one of claims 44-46, wherein the fungal infection comprises Aspergillus spp.

48. The method of claim 47, wherein the Aspergillus fungi comprise Aspergillus flavus, Aspergillus fumigatus, Aspergillus nidulans, Aspergillus oryzae, Aspergillus sydowii, Aspergillus versicolor, Aspergillus wentii, and/or Aspergillus niger.

49. The method of any one of claims 44-48, wherein the fungal infection comprises Candida spp.

50. The method of claim 49, wherein the Candida fungi comprise Candida albicans, Candida tropicalis, Candida glabrata, Candida auris, Candida lusitaniae, Candida parapsilosis, Candida krusei, Candida dubliniensis, and/or Candida guilliermondii.

51. The method of any one of claims 43-50, wherein the Dkk-1 inhibitors inhibit Dkk-1 activity.

52. The method of any one of claims 43-51, wherein the Dkk-1 inhibitors inhibit release of Dkk-1 from platelets.

53. The method of any one of claims 43-52, wherein the one or more Dkk-1 inhibitors comprise a small molecule, an antibody, or a nucleic acid.

54. The method of any one of claims 43-53, wherein the one or more Dkk-1 inhibitors comprise WAY 262611, gallocyanine, NCI8642, or functional derivatives thereof.

55. The method of any one of claims 43-54, wherein the one or more Dkk-1 inhibitors comprise WAY 262611 or a functional derivative thereof.

56. The method of any one of claims 43-55, wherein the one or more Dkk-1 inhibitors comprise DKN-01 or a functional derivative thereof.

57. The method of any one of claims 43-56, wherein the allergic airway disease is resistant to treatment with a corticosteroid.

58. The method of any one of claims 43-57, wherein inhibiting the adaptive or innate immune response in the airway of the subject comprises inhibiting cytokine secretion and/or inhibiting recruitment or activity of inflammatory cells and/or T helper effector cells.

59. The method of claim 58, wherein the cytokines comprise interleukin-2, interleukin-4, interleukin-5, interleukin-6, interleukin-9, interleukin-10, interleukin-12, interleukin-13, interleukin-17A, interleukin-17B, interleukin-17C, interleukin-17D, interleukin-17E, interleukin-17F, interleukin-22, interleukin-33, tumor necrosis factor, thymic stromal lymphopoietin, ciliary neurotrophic factor, or interleukin-1β.

60. The method of claim 58 or claim 59, wherein the inflammatory cells comprise granulocytes and/or macrophages.

61. The method of claim 60, wherein the T helper effector cells comprise T helper type 2 (Th2) cells and/or T helper type 17 (Th17) cells.

62. The method of any one of claims 43-61, wherein the composition further comprises one or more pharmaceutically acceptable excipients.

63. The method of any one of claims 43-62, wherein the composition is administered intranasally, subcutaneously, intravenously, by aerosol, and/or orally.

64. The method of any one of claims 43-63, wherein the subject is provided an effective amount one or more additional allergic airway disease therapies.

65. The method of claim 64, wherein the one or more additional therapies comprise corticosteroids, leukotriene modifiers, bronchodilators, antifungals, biologics, allergy shots, antihistamines, decongestants, cromolyn, or combinations thereof.

66. The method of claim 64, wherein the corticosteroids comprise fluticasone, dexamethasone, budesonide, mometasone, beclomethasone, ciclesonide, or combinations or derivatives thereof.

67. The method of claim 64, wherein the leukotriene modifiers comprise montelukast, zafirlukast, zileuton, or combinations or derivatives thereof.

68. The method of claim 64, wherein the bronchodilators comprise theophylline, albuterol, levalbuterol, ipratropium, aclidinium, arformoterol, formoterol, indacaterol, tiotropium, salmeterol, glycopyrrolate, olodaterol, vilanterol, umeclidinium, or combinations or derivatives thereof.

69. The method of claim 64, wherein the antifungals comprise amphotericin B, azithromycin, terbinafine, voriconazole, itraconazole, fluconazole, isavuconazole, posaconazole, ketoconazole, micafungin, ibrexafungerp, caspofungin, or combinations or derivatives thereof.

70. The method of claim 64, wherein the biologics comprise omalizumab, mepolizumab, benralizumab, dupilumab, reslizumab, or combinations or derivatives thereof.

71. The method of claim 64, wherein the antihistamines comprise azelastine, brompheniramine, cetirizine, chlorpheniramine, desloratadine, diphenhydramine, doxylamine, fexofenadine, hydroxyzine, ketotifen, loratadine, levocetirizine, olaptadine, or combinations or derivatives thereof.

72. The method of claim 64, wherein the decongestants comprise levmetamfetamine, naphazoline, pseudoephedrine, phenylephrine, propylhexedrine, oxymetazoline, xylometazoline, or combinations or derivatives thereof.

73. The method of any one of claims 64-72, wherein the one or more Dkk-1 inhibitors and one or more additional therapies are administered in the same composition.

74. The method of any one of claims 64-72, wherein the one or more Dkk-1 inhibitors and one or more additional therapies are administered in different compositions.

75. The method of any one of claims 43-74, wherein the composition is administered once or multiple times.

76. The method of claim 75, wherein when the composition is administered to the individual multiple times, the duration between administrations is within 1-24 hours, 1-7 days, 1-4 weeks, or 1-12 months.

77. The method of any one of claims 43-76, wherein the inflammation, hyper-responsiveness, and/or adaptive or innate immune response are symptoms of an allergic airway disease.

78. The method of claim 77, wherein the allergic airway disease comprises chronic rhinosinusitis, asthma, allergic bronchopulmonary mycosis, acute or chronic eosinophilic pneumonitis, Löffler's syndrome, eosinophilic granulomatosis with polyangiitis, chronic obstructive pulmonary disease with airway mycosis, interstitial lung disease with airway mycosis, chronic pulmonary aspergillosis, pulmonary or sinus aspergilloma, hypersensitivity pneumonitis, extrinsic allergic alveolitis, or a combination thereof.

79. The method of claim 77 or claim 78, wherein the allergic airway disease comprises asthma.

80. A composition comprising:
(a) one or more Dkk-1 inhibitors; and
(b) one or more allergic airway disease therapies.

81. The composition of claim 80, wherein the one or more Dkk-1 inhibitors comprise a small molecule, an antibody, or a nucleic acid.

82. The composition of claim 80 or claim 81, wherein the one or more Dkk-1 inhibitors comprise WAY 262611, gallocyanine, NCI8642, or functional derivatives thereof.

83. The composition of any one of claims 80-82, wherein the one or more Dkk-1 inhibitors comprise WAY 262611 or a functional derivative thereof.

84. The composition of any one of claims 80-83, wherein the one or more Dkk-1 inhibitors comprise DKN-01 or a functional derivative thereof.

85. The composition of any one of claims 80-84, wherein the one or more allergic airway disease therapies comprise corticosteroids, leukotriene modifiers, bronchodilators, antifungals, biologics, allergy shots, antihistamines, decongestants, cromolyn, or combinations thereof.

86. The composition of claim 85, wherein the corticosteroids comprise fluticasone, dexamethasone, budesonide, mometasone, beclomethasone, ciclesonide, or combinations or derivatives thereof.

87. The composition of claim 85, wherein the leukotriene modifiers comprise montelukast, zafirlukast, or zileuton.

88. The composition of claim 85, wherein the bronchodilators comprise theophylline, albuterol, levalbuterol, ipratropium, aclidinium, arformoterol, formoterol, indacaterol, tiotropium, salmeterol, glycopyrrolate, olodaterol, vilanterol, umeclidinium, or combinations or derivatives thereof.

89. The method of claim 85, wherein the antifungals comprise amphotericin B, azithromycin, terbinafine, voriconazole, itraconazole, fluconazole, isavuconazole, posaconazole, ketoconazole, micafungin, ibrexafungerp, caspofungin, or combinations or derivatives thereof.

90. The composition of claim 85, wherein the biologics comprise omalizumab, mepolizumab, benralizumab, dupilumab, reslizumab, or combinations or derivatives thereof.

91. The composition of claim 85, wherein the antihistamines comprise azelastine, brompheniramine, cetirizine, chlorpheniramine, desloratadine, diphenhydramine, doxylamine, fexofenadine, hydroxyzine, ketotifen, loratadine, levocetirizine, olaptadine, or combinations or derivatives thereof.

92. The composition of claim 85, wherein the decongestants comprise levmetamfetamine, naphazoline, pseudoephedrine, phenylephrine, propylhexedrine, oxymetazoline, xylometazoline, or combinations or derivatives thereof.

93. The composition of any one of claims 80-93, wherein the one or more Dkk-1 inhibitors and the one or more allergic airway disease therapies are in different formulations.

94. The composition of any one of claims 80-93, wherein the one or more Dkk-1 inhibitors and the one or more allergic airway disease therapies are in the same formulation.

95. The composition of any one of claims 80-94, wherein the composition further comprises one or more pharmaceutically acceptable excipients.
